(19) United States
(12) Patent Application Publication
(10) Pub. No.: US 2023/0233566 A1
(43) Pub. Date: Jul. 27, 2023

(54) INHIBITION OF BMI1 ELIMINATES CANCER STEM CELLS AND ACTIVATES ANTITUMOR IMMUNITY

(71) Applicant: The Regents of the University of California, Oakland, CA (US)

(72) Inventors: Cun-Yu WANG, Los Angeles, CA (US); Lingfei JIA, Los Angeles, CA (US)

(73) Assignee: The Regents of the University of California, Oakland, CA (US)

(21) Appl. No.: 17/928,767

(22) PCT Filed: Jun. 3, 2021
(86) PCT No.: PCT/US21/35735

§ 371 Date: Nov. 30, 2022

Related U.S. Application Data
(60) Provisional application No. 63/034,818, filed on Jun. 4, 2020.

(51) Int. Cl. A61K 31/519; A61K 39/395; A61K 31/506; A61K 31/4406; A61K 31/497; C12N 15/113; A61P 35/04; A61K 33/243

(52) U.S. Cl. CPC A61K 31/519; A61K 39/39533; A61K 31/506; A61K 31/4406; A61K 31/497; C12N 15/1135; A61P 35/04; A61K 33/243; C12N 2310/14

(57) ABSTRACT

The present disclosure reports that pharmacological or genetic inhibition of Moloney murine leukemia virus insertion site 1 (BMI1) not only helped to eliminate BMI1+ cancer stem cells (CSCs), but can also augment PD1 blockade by strongly induced tumor cell-intrinsic immune responses by recruiting and activating CD8+ T cells. Taken together, the results indicate that in addition to purging CSCs, targeting BMI1 would enable immune checkpoint blockade to inhibit metastatic tumor growth and prevent tumor relapse by activating cell-intrinsic immunity.

### CROSS-REFERENCE TO RELATED APPLICATION

This application claims priority to U.S. provisional patent application Ser. No. 63/034,818, filed Jun. 4, 2020, the entire contents of which is incorporated herein by reference.

### STATEMENT REGARDING FEDERALLY SPONSORED RESEARCH

This invention was made with government support under Grant Numbers DE015964 and CA236878 awarded by the National Institutes of Health. The government has certain rights in the invention.

### FIELD OF THE INVENTION

The present disclosure relates generally to the field of cancer immunotherapy. In one embodiment, the present disclosure describes targeting Moloney murine leukemia virus insertion site 1 (BMI1) would enable immune checkpoint blockade to inhibit metastatic tumor growth and prevent tumor relapse by activating cell-intrinsic immunity.

### BACKGROUND OF THE INVENTION

Cancer stem cells (CSCs), also known as cancer initiating cells, are associated with tumor initiation, growth and metastasis. Growing evidence suggest that CSCs might be responsible for cancer therapy resistance and relapse or recurrence. In order to achieve complete regression of tumors, CSCs have to be targeted based on the CSC theory. Moloney murine leukemia virus insertion site 1 (BMI1) has been found to control CSC self-renewal and functions in several human cancers including head and neck squamous cell carcinoma (HNSCC). BMI1 is a core component of the polycomb repressive complex 1 (PRC1) that mediates gene silencing via monoubiquitination of histone H2A. Targeting BMI1 with the small molecule inhibitor PTC209 was shown to abolish the self-renewal of CSCs isolated from human colorectal cancers in the xenografted nude mouse model.

HNSCC is an aggressive malignancy with a low 5-year survival rate and poor prognosis. It is highly invasive and frequently metastasizes to cervical lymph nodes. Programmed cell death protein 1 (PD1) blockade combined with chemotherapy has been approved for treating recurrent or metastatic HNSCC and has significantly changed the therapeutic landscape of HNSCC. Unfortunately, the objective responsive rates are not very high and the median response duration is relatively short, indicating that HNSCC might be intrinsically resistant to PD1 blockade and eventually relapse after treatment. Because CSCs were often defined by using immunodeficient mouse models, it is largely unknown whether PD1 blockade-based immunotherapy can target CSCs. Growing evidence suggests that CSCs may secrete various growth factors and cytokines to inhibit immune responses and promote immunosuppressive tumor microenvironment. The expression of the antigen processing and major histocompatibility complex molecules has been found to be downregulated in CSCs of glioblastoma and prostate cancer. On the other hand, PD-L1 was shown to be elevated in CSCs of human HNSCC and other solid tumors. Very recently, it has been shown that CSCs directly inhibited cytotoxic T cell activity and mediated tumor resistance to adoptive cytotoxic T cell transfer-based immunotherapy by expressing CD80. Taken together, these studies suggest that targeting CSCs may be critical for improving the efficacy of immunotherapy and preventing tumor relapses.

Despite there are exciting progresses in cancer immunotherapy, currently, there are no pre-clinical or clinical studies to show that immune checkpoint blockade can eliminate cancer stem cells (CSCs) by activating anti-tumor immunity. Thus, there is a need for further studies on immunotherapy related to targeting CSCs and BMI1.

### SUMMARY OF THE INVENTION

Recently, a 4 nitroquinoline-1 oxide (4NQO) induced Bmi1^{CreER};Rosa^{tdTomato} mouse model for head and neck squamous cell carcinoma (HNSCC) was established, which fully mimics human HNSCC development and metastasis and allows one to perform in vivo lineage tracing of BMI1⁺ CSCs in an unperturbed tumor immune microenvironment. Taking advantage of this model, it was investigated whether BMI1⁺ CSCs could be eradicated by PD1 blockade-based combination therapy. Unexpectedly, it is found that pharmacological or genetic inhibition of Moloney murine leukemia virus insertion site 1 (BMI1) not only helped to eliminate BMI1⁺ CSCs, but also augment PD1 blockade by activating tumor cell-intrinsic immunity, resulting in inhibition of metastatic tumor growth and prevention of tumor relapse. These preclinical studies provide an important foundation for developing new clinical trial for PD1 blockade-based combination therapy with BMI1 inhibitors.

In one embodiment, the present disclosure provides a method of treating cancer in a patient in need thereof, comprising the steps of (i) administering to the patient an agent that blocks signaling through programmed cell death protein 1 (PD-1); and (ii) administering to the patient an agent that reduces expression or function of B-cell-specific Moloney murine leukemia virus integration site 1 (BMI-1). In one embodiment, the method reduces cancer metastasis. In another embodiment, the method reduces the number of BMI-1⁺ cancer stem cells.

In one embodiment, the present disclosure provides a method of increasing anti-tumor T cell activities in a patient having a cancer, comprising the steps of (i) administering to the patient an agent that blocks signaling through programmed cell death protein 1 (PD-1); and (ii) administering to the patient an agent that reduces expression or function of B-cell-specific Moloney murine leukemia virus integration site 1 (BMI-1). In one embodiment, the method increases anti-tumor T cell activities that are mediated by CD8+ T cells.

In one embodiment, the present disclosure provides a method of reducing the number of cancer stem cells in a cancer patient in need thereof, comprising the steps of (i) administering to the patient an agent that blocks signaling through programmed cell death protein 1 (PD-1); and (ii) administering to the patient an agent that reduces expression or function of B-cell-specific Moloney murine leukemia virus integration site 1 (BMI-1). In one embodiment, the method cancer stem cells are BMI1⁺.

In one embodiment, there is provided a composition for treating cancer in a patient, comprising (i) an agent that blocks signaling through programmed cell death protein 1 (PD-1); and (ii) an agent that reduces expression or function of B-cell-specific Moloney murine leukemia virus integration site 1 (BMI-1).

These and other aspects of the invention will be appreciated from the ensuing descriptions of the figures and detailed description of the invention.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1M show enrichment of BMI1⁺ CSCs after combination treatment of anti-PD1 and cisplatin. FIG. 1A presents schematic diagrams showing the treatment and lineage tracing of primary HNSCC in Bmi1^{CreER};Rosa^{tdTomato} mice. Tamoxifen (Tam) was administered 1 day prior to sacrificing (Sac) the mice in order to label BMI1⁺ CSCs. FIG. 1B presents representative image of tongue visible lesions in different treatment groups. Black dashed lines demark lesion areas. Scale bar, 2 mm. FIG. 1C shows quantification of HNSCC lesion area from mice with treatment as indicated. Values are mean±SD from the pool of two independent experiments. n=10. *p<0.05 and **p<0.01 by one-way ANOVA. FIG. 1D shows representative H&E staining of HNSCC from mice with treatment as indicated. Scale bar, 200 μm. Enlarged images are shown in the lower panels. Scale bar, 50 μm. FIG. 1E shows quantification of HNSCC number and area from mice with treatment as indicated. Values are mean±SD from the pool of two independent experiments. n=10. *p<0.05 and **p<0.01 by one-way ANOVA. FIG. 1F shows quantification of HNSCC invasion grades from mice with treatment as indicated. Stacked bars show Grade 3 (top) over Grade 2 over Grade 1 (bottom). Data was pooled from two independent experiments. n=10. *p<0.05 by Cochran-Armitage test. FIG. 1G shows immunostaining of metastatic cells in cervical lymph nodes using anti-PCK. Scale bar, 200 μm. FIG. 1H shows percentage of metastatic lymph nodes from mice with treatment as indicated. Number of metastatic lymph nodes in each group is indicated in the figure. Data was pooled from two independent experiments. *p<0.05 by Chi-square test. FIG. 1I shows quantification of metastatic area in lymph nodes from mice with treatment as indicated. Values are mean±SEM from the pool of two independent experiments. *p<0.05 by one-way ANOVA. FIG. 1J shows immunofluorescent images for CD8⁺ T from mice with treatment as indicated. Scale bar, 10 μm. FIG. 1K shows quantifications of CD8⁺ T cells in HNSCC from mice with treatment as indicated. Values are mean±SD from the pool of two independent experiments. n=10, **p<0.01 by one-way ANOVA. FIG. 1L shows representative images of Tomato⁺ BMI1⁺ CSCs in HNSCC from mice with treatment as indicated. White dashed lines demark tumor-stromal junction. Scale bar, 10 μm. FIG. 1M shows quantification of the percentage of Tomato⁺ cells in HNSCC from mice with treatment as indicated. Values are mean±SD from the pool of two independent experiments. n=10. *p<0.05 and **p<0.01 by one-way ANOVA.

FIGS. 2A-2N show PTC-209 eliminates BMI1⁺ CSCs and collaborates with anti-PD1 to suppress HNSCC growth and metastasis by recruiting CD8+ cells. FIG. 2A shows representative image of tongue visible lesions in different treatment groups as indicated. Black dashed lines demark lesion areas. Scale bar, 2 mm. FIG. 2B shows quantification of HNSCC lesion areas from mice. Values are mean±SD from the pool of two independent experiments. n=12, *p<0.05 and **p<0.01 by one-way ANOVA. FIG. 2C shows representative H&E staining of HNSCC from mice with treatment as indicated. Scale bar, 200 μm. Enlarged images are shown in the lower panels. Scale bar, 50 μm. FIG. 2D shows quantification of HNSCC number and area. Values are mean±SD from the pool of two independent experiments. n=12, *p<0.05 and **p<0.01 by one-way ANOVA. FIG. 2E shows quantification of HNSCC invasion grades. Stacked bars show Grade 3 (top) over Grade 2 over Grade 1 (bottom); the PTC209+Anti-PD1 data are Grade 2 (top) over Grade 1 (bottom). *p<0.05 and **p<0.01 by Cochran-Armitage test. FIG. 2F shows representative images of active caspase3 (Ac-casp3, green) in HNSCC. Nuclei were stained with DAPI (blue). White dashed lines demark tumor-stromal junction. Scale bar, 10 μm. FIG. 2G shows percentage of Ac-Casp3⁺ cells in HNSCC from mice with indicated treatments. Values are mean±SD from the pool of two independent experiments. n=12, **p<0.01 by one-way ANOVA. FIG. 2H shows immunostaining of metastatic cells in cervical lymph nodes by anti-PCK. Scale bar, 200 μm. FIG. 2I shows quantification of percentage of metastatic lymph nodes from mice with treatment as indicated. Number of metastatic lymph nodes in each group is indicated in the figure. *p<0.05 and **p<0.01 by Chi-square test. FIG. 2J shows quantification of metastatic areas in lymph nodes from mice with treatment as indicated. Values are mean±SEM from the pool of two independent experiments. *p<0.05 and **p<0.01 by one-way ANOVA. FIG. 2K shows representative immunofluorescent images for CD8 (red) and PCK (green) of HNSCC from mice with treatment as indicated. Nuclei were visualized by DAPI (blue). Scale bar, 10 μm. FIG. 2L shows quantifications of CD8+ T cells percentage from mice with treatment as indicated. Values are mean±SD from the pool of two independent experiments. n=12, **p<0.01 by one-way ANOVA. FIG. 2M shows representative images of Bmi1⁺ cell-driven lineage tracing in HNSCC from mice with treatment as indicated. White dashed lines demark tumor-stromal junction. Scale bar, 10 μm. FIG. 2N shows quantification of the percentage of Tomato⁺ cells in HNSCC from mice with treatment as indicated. Values are mean±SD from the pool of two independent experiments. n=12, *p<0.05 and **p<0.01 by one-way ANOVA.

FIGS. 3A-3L show depletion of intratumoral CD8+ T cells reverse PTC209 plus anti-PD1-mediated anti-tumor immunity. FIG. 3A shows representative immunofluorescent images for CD8 (red) and PCK (green) in HNSCC from mice with indicated treatments. Nuclei were visualized by DAPI (Blue). Scale bar, 10 μm. FIG. 3B shows quantifications of percentage of CD8⁺ T cells in HNSCC. Values are mean±SD from the pool of two independent experiments. n=8, *p<0.05 and **p<0.01 by one-way ANOVA. FIG. 3C shows representative image of tongue visible lesions. Scale bar, 2 mm. FIG. 3D shows quantification of HNSCC lesion areas. Values are mean±SD from the pool of two independent experiments. n=8, **p<0.01 by one-way ANOVA. FIG. 3E shows representative H&E staining of HNSCC. Scale bar, 200 μm. Enlarged images are shown in the lower panels. Scale bar, 50 μm. FIG. 3F shows quantification of HNSCC number and area. Values are mean±SD from the pool of two independent experiments. n=8, *p<0.05 and **p<0.01 by one-way ANOVA. FIG. 3G shows quantification of HNSCC invasion grades. Stacked bars show Grade 3 (top) over Grade 2 over Grade 1 (bottom); PCT209+Anti-PD1 data are Grade 2 (top) over Grade 1 (bottom). *p<0.05 by Cochran-Armitage test. FIG. 3H shows representative images of Ac-casp3 (red) and PCK (green) in HNSCC. Nuclei were visualized by DAPI (Blue). Scale bar, 10 μm. FIG. 3I shows percentage of Ac-casp3⁺ apoptotic cells in all tumor cells. Values are mean±SD from the pool of two independent experiments. n=8, **p<0.01 by one-way ANOVA. FIG. 3J shows immunostaining of metastatic cells in cervical lymph nodes using anti-PCK. Scale bar, 200 μm. FIG. 3K shows percentage of metastatic lymph nodes from mice. Number of metastatic lymph nodes in each group is indicated in the figure. *p<0.05 and **p<0.01 by Chi-square test. FIG. 3L shows quantification of metastatic areas in cervical lymph nodes. Values are mean±SEM from the pool of two independent experiments. *p<0.05 and **p<0.01 by one-way ANOVA.

FIGS. 4A-4K show epithelial deletion of BMI1 collaborates with anti-PD1 to suppress HNSCC growth and metastasis by recruiting CD8⁺ cells. FIG. 4A shows experimental design for Bmi1 knockout in tumor cells and anti-PD1 treatment in vivo. Three administrations of Tam were given to tumor-bearing mice. Mice were randomly divided into four experimental groups (n=14 per group from two independent experiments): BMI1^{f/f} with IgG isotype, BMI1^{f/f} with anti-PD1, K14Cre;BMI1^{f/} with IgG isotype, and K14Cre;BMI1^{f/f} with anti-PD1. FIG. 4B shows representative image of tongue visible lesions. Black dashed lines demark lesion area. Scale bar, 2 mm. FIG. 4C shows quantification of lesion areas from mice treated with different conditions as indicated. Values are mean±SD from the pool of two independent experiments. *p<0.05 and **p<0.01 by two-way ANOVA. FIG. 4D shows representative H&E staining of HNSCC from mice treated with different conditions as indicated. Scale bar, 200 μm. Enlarged images are shown in the lower panels. Scale bar, 50 μm. FIG. 4E shows quantification of HNSCC area and number from mice treated with different conditions as indicated. Values are mean±SD from the pool of two independent experiments. *p<0.05 and **p<0.01 by two-way ANOVA. FIG. 4F shows quantification of HNSCC invasion grades from mice treated with different conditions as indicated. Stacked bars show Grade 3 (top) over Grade 2 over Grade 1 (bottom); data for K14Cre; Bmi1^{f/f} with anti-PD1 are Grade 2 (top) over Grade 1 (bottom). *p<0.05 and **p<0.01 by Cochran-Armitage test. FIG. 4G shows representative immunostaining of metastatic cells in cervical lymph nodes by anti-PCK. Scale bar, 200 μm. FIG. 4H shows quantification of percentage of metastatic lymph nodes. Number of metastatic lymph nodes in each group is indicated in the figure. For each of isotype and Anti-PD1, the left bar is Bmi1^{f/f}, and the right bar, K14Cre; Bmi1^{f/f}. *p<0.05 and **p<0.01 by Chi-square test. FIG. 4I shows quantification of metastatic area in lymph nodes from mice treated with different conditions as indicated. Values are mean±SEM from the pool of two independent experiments. *p<0.05 and **p<0.01 by two-way ANOVA. FIG. 4J shows representative immunofluorescent images for CD8 (red) and PCK (green) in HNSCC. Nuclei were visualized by DAPI (blue). Scale bar, 10 μm. FIG. 4K shows quantification of the percentage of CD8⁺ T cells from mice treated with different conditions as indicated. For each of isotype and Anti-PD1, the left bar is Bmi1^{f/f}, and the right bar, K14Cre; Bmi1^{f/f}. Values are mean±SD from the pool of two independent experiments. **p<0.01 by two-way ANOVA. ##p<0.01 treatment×genotype interaction.

FIGS. 5A-5J show BMI1 Inhibition induces expression of effector T cell attracting chemokines in SCC cells by activating cGAS-STING-IRF3 signaling and erasing repressive H2AUb on their promoters. FIG. 5A shows heatmap from RNA-sequencing data showing the differentially expressed genes related to chemokines-mediated signaling in SCC23 cells upon PTC209 or BMI1 knockdown. Blue rectangles indicate the genes related to IFN-regulated chemokines. FIG. 5B shows results of qRT-PCR indicating that the expression of CCL5, CXCL9, CXCL10, and CXCL11 in SCC23 cells were induced by PTC209 or BMI1 knockdown. For SCC23, for each chemokine, the left bar is DMSO (top graph) or shCtrl (bottom graph), and the right bar is PTC209 (top graph) or shBMI1 (bottom graph). For Means±SD were shown. **p<0.01 by unpaired Student's t test. FIG. 5C shows immunofluorescent staining of pH2A.X (green) in 4NQO-induced HNSCC by PTC209 or BMI1 knockout and their quantifications. Nuclei were stained with DAPI (blue). For 4NQO, the left bar is vehicle (top graph) or Bmi1^{f/f} (bottom graph) and the right bar, PTC209 (top graph) or K14Cre; Bmi1^{f/f}. Scale bar, 10 μm. Means±SD were shown (n=8). **p<0.01 by unpaired Student's t test. FIG. 5D shows confocal images showing cytosolic DNA accumulations and their quantifications in SCC23 cells upon PTC209 or shBMI1 treatment. Double strand DNA (dsDNA) was stained by Picogreen (green). Mitochondria and nuclei were respectively stained with Mito-tracker (Red) and DAPI (blue). White arrows indicate cytosolic dsDNA. Left bar in above graph, DMSO, right bar PTC209. Left bar in below graph shCtrl; right bar shBMI1. Scale bar, 10 μm. More than 100 cells were analyzed per group. Means±SD were shown. **p<0.01 by one-way ANOVA.

FIG. 5E shows induction of phosphorylation of STING (S366), TBK1 (S172) and IRF3 (S396) in SCC23 cells by PTC209 or shBMi1 treatment. FIG. 5F presents results of qRT-PCR showing the induction of IFNβ mRNA expression in SCC23 cells by PTC209 or shBMI1 treatment. Left bar in left graph, DMSO, right bar PTC209. Left bar in right graph siCtrl; right bar shBMI1. **p<0.01 by unpaired Student's t test. FIG. 5G shows reduction of BMI1 occupied on the promoters of CCL5, CXCL9, CXCL10, and CXCL11 in SCC23 cells by PTC209. For each pair or bars, left is Bmi1 and right is IgG. FIG. 5H shows reduction of H2AUb levels on the promoters of CCL5, CXCL9, CXCL10, and CXCL11 in SCC23 cells by PTC209. For each pair or bars, left is H2AK119ub, and right is IgG. FIG. 5I shows reduction of BMI1 occupied on the promoters of CCL5, CXCL9, CXCL10, and CXCL11 in SCC23 cells by shBMI1. For each pair or bars, left is Bmi1 and right is IgG. FIG. 5J shows reduction of H2AUb levels on the promoters of CCL5, CXCL9, CXCL10, and CXCL11 in SCC23 cells by shBMI1. For each pair or bars, left is H2AK119ub, and right is IgG. n=3, means±SD are shown. *p<0.05 and **p<0.01 by unpaired Student's t test.

FIGS. 6A-6L show inhibition of chemokine signaling impairs PTC209 plus anti-PD1-mediated anti-tumor immunity. FIG. 6A shows representative immunofluorescent images for CD8 (red) and PCK (green) in HNSCC from mice with indicated treatments. Nuclei were visualized by DAPI (Blue). Scale bar, 10 μm. FIG. 6B shows quantifications of percentage of CD8+ T cells in HNSCC. Values are mean±SD from the pool of two independent experiments. n=8, *p<0.05 and **p<0.01 by one-way ANOVA. FIG. 6C shows representative image of tongue visible lesions. Scale bar, 2 mm. FIG. 6D shows quantification of HNSCC lesion areas. Values are mean±SD from the pool of two independent experiments. n=8, *p<0.05 and **p<0.01 by one-way ANOVA. FIG. 6E shows representative H&E staining of HNSCC. Scale bar, 200 μm. Enlarged images are shown in the lower panels. Scale bar, 50 μm. FIG. 6F shows quantification of HNSCC number and area. Values are mean±SD from the pool of two independent experiments. n=8, *p<0.05 and **p<0.01 by one-way ANOVA. FIG. 6G shows quantification of HNSCC invasion grades. Stacked bars show Grade 3 (top) over Grade 2 over Grade 1 (bottom); data for PTC209+anti-PD1 are Grade 2 (top) over Grade 1 (bottom). n=8, *p<0.05 by Cochran-Armitage test. FIG. 6H shows representative images of Ac-casp3 (red) and PCK (green) in HNSCC. Nuclei were visualized by DAPI (Blue). Scale bar, 10 μm. FIG. 6I shows percentage of Ac-casp3⁺ apoptotic cells in all tumor cells. Values are mean±SD from the pool of two independent experiments. n=8, *p<0.05 and **p<0.01 by one-way ANOVA. FIG. 6J shows immunostaining of metastatic cells in cervical lymph nodes using anti-PCK. Scale bar, 200 μm. FIG. 6K shows percentage of metastatic lymph nodes from mice. Number of metastatic lymph nodes in each group is indicated in the figure. *p<0.05 and **p<0.01 by Chi-square test. FIG. 6L shows quantification of metastatic areas in cervical lymph nodes. Values are mean±SEM from the pool of two independent experiments. *p<0.05 and **p<0.01 by one-way ANOVA.

FIGS. 7A-7Q show the combination treatment of anti-PD1 and PTC209 prevents BMI1⁺ CSC-mediated tumor relapse. FIG. 7A shows the experimental design for BMI1⁺ CSCs lineage tracing in HNSCC after treatment with anti-PD1 plus cisplatin or anti-PD1 plus PTC209 (n=7 per group). After treatment, mice were injected with Tam and maintained for 4 additional weeks. FIG. 7B shows representative images of Tomato⁺ tumor cells (red) derived from BMI1⁺ CSCs one month after treatment. Nuclei are stained with DAPI (blue). White dashed lines demark tumor-stromal junction. Scale bar, 10 μm. FIG. 7C shows quantification of the percentage of Tomato⁺ tumor cells in HNSCC. Values are mean±SD from the pool of two independent experiments. ns, not significant, *p<0.05 and **p<0.01 by one-way ANOVA. FIG. 7D shows quantification of HNSCC lesion areas. Values are mean±SD from the pool of two independent experiments. ns, not significant, *p<0.05 and **p<0.01 by one-way ANOVA. FIG. 7E shows quantification of HNSCC number and area from mice with treatment as indicated. Values are mean±SD from the pool of two independent experiments. ns, not significant, *p<0.05 and **p<0.01 by one-way ANOVA. FIG. 7F shows quantification of HNSCC invasion grades. ns, not significant, *p<0.05 by Cochran-Armitage test. Stacked bars show Grade 3 (top) over Grade 2 over Grade 1 (bottom); data for PTC209+anti-PD1 are Grade 2 (top) over Grade 1 (bottom). FIG. 7G shows experimental design for examining HNSCC relapse after treatment. Bmi^{CreER};Rosa^{tdTomato} mice with 4NQO induced HNSCC were randomly divided into three experimental groups (n=8). After treatment, mice were maintained for 8 additional weeks for the tumor relapse. Tamoxifen (Tam) was administered 1 day prior to sacrificing (Sac) the mice in order to label BMI1⁺ CSCs. FIG. 7H shows representative image of tongue visible lesions. Black dashed lines demark lesion area. Scale bar, 2 mm. FIG. 7I shows quantification of HNSCC lesion areas. Mean±SD from the pool of two independent experiments. ns, not significant, *p<0.05 and **p<0.01 by one-way ANOVA. FIG. 7J shows H&E staining of HNSCC. Scale bar, 200 μm. Enlarged images are shown in the lower panels. Scale bar, 50 μm. FIG. 7K shows quantification of HNSCC number and area. Mean±SD from the pool of two independent experiments. ns, not significant, *p<0.05 and **p<0.01 by one-way ANOVA. FIG. 7L shows quantification of HNSCC invasion grades. ns, not significant, *p<0.05 by Cochran-Armitage test. Stacked bars show Grade 3 (top) over Grade 2 over Grade 1 (bottom); data for PTC209+anti-PD1 are Grade 2 (top) over Grade 1 (bottom). FIG. 7M shows immunostaining of metastatic cells in cervical lymph nodes by anti-PCK. Scale bar, 200 μm. FIG. 7N shows percentage of metastatic lymph nodes in HNSCC. Number of metastatic lymph nodes in each group is indicated. ns, not significant, **p<0.01 by Chi-square test. FIG. 7O shows quantification of metastatic areas in lymph nodes. Mean±SEM from the pool of two independent experiments. ns, not significant, *p<0.05 and **p<0.01 by one-way ANOVA. FIG. 7P shows representative images of Tomato⁺ BMI1⁺ CSCs in HNSCC after treatment. White dashed lines demark tumor-stromal junction. Scale bar, 10 μm. FIG. 7Q shows quantification of the percentage of Tomato⁺ BMI1⁺ CSCs in HNSCC after treatment. Mean±SD from the pool of two independent experiments. *p<0.05 and **p<0.01 by one-way ANOVA.

FIGS. 8A-8B show Cisplatin plus anti-PD1 induces apoptosis in HNSCC. FIG. 8A shows representative staining for Ac-casp3⁺ apoptotic cells (green) and Tomato⁺ CSCs (red) in HNSCC. Nuclei were stained with DAPI (blue). White dashed lines demark tumor-stromal junction. Scale bar, 10 μm. FIG. 8B shows quantifications of percentage of Ac-casp3⁺ cells in Tomato⁺ (right bar of each pair or not determined [n.d.]) and Tomato⁻ (left bar of each pair) cells in HNSCC. Mean±SD from the pool of two independent experiments (n=8). nd, not detected. **p<0.01 by one-way ANOVA.

FIGS. 9A-9F show PTC209 treatment does not affect CD8⁺ cell activation in lymph nodes, blood and spleen of mice. FIG. 9A presents results of immunochemistry showing the inhibition of BMI1 expression in HNSCC by PTC209. Scale bar, 10 μm. FIG. 9B shows results of Western blot showing the inhibition of BMI1 expression in HNSCC tumor tissues by PTC209 treatment. GAPDH was used as internal control. FIG. 9C shows flow cytometry analysis of CD8⁺ and CD4⁺ T cells in cervical lymph nodes, blood, and spleen of 4NQO-induced HNSCC mice treated with vehicle and PTC209. FIG. 9D shows quantifications of percentage of CD8⁺ and CD4⁺ T cells in CD3⁺ cells in lymph nodes, blood, and spleen of each group as indicated. For each pair of bars, left is Vehicle and right is PTC209. Values are mean±SD. n=6, ns, not significant by unpaired Student's t test. FIG. 9E shows flow cytometry analysis of percentages of CD8⁺ T cells expressing IFNγ in cervical lymph nodes, blood, and spleen of 4NQO-induced HNSCC mice treated with vehicle or PTC209. FIG. 9F shows quantifications of percentage of CD8+ T cells secreting IFNγ in lymph nodes, blood, and spleen of each group as indicated. For each pair of bars, left is Vehicle and right is PTC209. Values are mean±SD, n=6, ns, not significant by unpaired Student's t test.

FIGS. 10A-10G show anti-PD1 plus PTC209 recruits and activates CD8⁺ cells in HNSCC. FIG. 10A shows representative images of Ac-casp3+(green) and Tomato⁺ CSCs (red) in HNSCC. Nuclei were stained with DAPI (blue). White dashed lines demark tumor-stromal junction. Scale bar, 10 μm. FIG. 10B shows percentage of Ac-casp3+ cells in Tomato⁻ cells in HNSCC from mice with treatment as indicated. Values are mean±SD from the pool of two independent experiments. n=12, **p<0.01 by one-way ANOVA. FIG. 10C shows quantifications of percentage of Ac-casp3⁺ in Tomato⁻ cells in HNSCC. Values are mean±SD from the pool of two independent experiments. n=12. nd, not detected. FIG. 10D shows representative immunofluorescent images for CD8+ T and GzmB⁺ T cells. The upper, middle and lower panels respectively show the staining of GzmB (green), CD8 (Red), and CD8 co-localization with GzmB. Nuclei were visualized by DAPI (blue). White dashed lines demark tumor-stromal junction. Scale bar, 10 μm. FIG. 10E shows quantifications of percentage of GzmB⁺ CD8⁺ T cells percentage in HNSCC. Values are mean±SD from the pool of two independent experiments. n=12. **p<0.01 by one-way ANOVA. FIG. 10F shows representative images of SOX2⁺ cells in HNSCC after treatment. SOX2 staining intensity was scored as: 1=weak; 2=moderate; 3=strong. Only tumor cells with strong SOX2 staining were counted as SOX2⁺ CSCs. Black arrows indicate SOX2⁺ CSCs. Scale bar, 10 μm. FIG. 10G shows quantification of the percentage of SOX2⁺ CSCs in HNSCC after treatment. Values are mean±SD from the pool of two independent experiments. n=12, *p<0.05 and **p<0.01 by one-way ANOVA.

FIGS. 11A-11F show tumor cell deletion of BMI1 collaborates with anti-PD1 to inhibit HNSCC by recruiting and activating CD8+ T cells. FIG. 11A shows immunochemical staining of BMI1 in HNSCC from both Bmi1^{f/f} and K14Cre;Bmi1^{f/f} mice after Tam treatment. Scale bar, 10 μm. FIG. 11B presents Western blot showing that BMI1 was deleted in HNSCC from K14Cre;Bmi1^{f/f}. FIG. 11C shows immunostaining of Ac-casp3+ apoptotic cells (green) in HNSCC. Nuclei were stained with DAPI (blue). For each pair of bars, left is Bmi1^{f/f}, and right is K14Cre;Bmi1^{f/f}. Scale bar, 10 μm. FIG. 11D shows percentage of Ac-casp3⁺ cells in HNSCC. Values are mean±SD from the pool of two independent experiments. n=14, *p<0.05 and **p<0.01 by two-way ANOVA. ##p<0.01 treatment×genotype interaction. FIG. 11E shows immunofluorescent staining of CD8⁺ and GzmB⁺ T cells. The upper, middle and lower panels respectively show the staining of GzmB (green), CD8 (Red), and CD8 co-localization with GzmB. Nuclei were visualized by DAPI (Blue). White dashed lines demark tumor-stromal junction. Scale bar, 10 μm. FIG. 11F shows quantifications of percentage of GzmB CD8+ T cells in HNSCC. Values are mean±SD from the pool of two independent experiments. For each pair of bars, left is Bmi1^{f/f}, and right is K14Cre;Bmi1^{f/f}. n=14, **p<0.01 by two-way ANOVA. ##p<0.01 treatment×genotype interaction.

FIGS. 12A-12F show BMI1 inhibition activates tumor cell-intrinsic anti-tumor immunity in SCC cells. FIG. 12A shows Western blot analysis of BMI1 and H2Aub in SCC23 and SCC1 treated with shBMI1 or PTC209. FIG. 12B shows histogram of the top 10 most-enriched GO terms of upregulated genes (fold change >2) in SCC23 cells induced by PTC209. FIG. 12C shows histogram of the top 10 most-enriched GO terms of upregulated genes (fold change >2) in SCC23 cells induced by shBMI1. FIG. 12D presents ELISA results showing protein levels of CCL5, CXCL9, CXCL10, and CXCL11 in SCC23 cells were induced by PTC209 or BMI1 knockdown. Means±SD were shown. **p<0.01 by unpaired Student's t test. FIG. 12E presents qRT-PCR showing the expression of CCL5, CXCL9, CXCL10, and CXCL11 in SCC1 cells were induced by PTC209 or BMI1 knockdown. In the graph above, for each pair of bars, left is DMSO and right is PTC209; in the graph below, for each pair of bars, left is shCtrl and right is shBMI1. Means±SD were shown. **p<0.01 by unpaired Student's t test. FIG. 12F presents ELISA results that showed the protein levels of CCL5, CXCL9, CXCL10, and CXCL11 in SCC1 cells were induced by PTC209 or BMI1 knockdown. Means±SD were shown. **p<0.01 by unpaired Student's t test.

FIGS. 13A-13C show BMI1 protein expression levels are negatively associated with the expression of CD8, CCL5, and CXCL10 in human HNSCC. FIG. 13A shows BMI1 protein expression levels were negatively correlated with the expression of CD8, CCL5, and CXCL10 in human HNSCC samples (n=60). The Pearson and Spearman correlation coefficient of liner regression was used to determine the correlation between different proteins. FIG. 13B shows representative immunostaining of human HNSCC samples with high BMI1 expression and corresponding low expression of CD8, CCL5, and CXCL10. Scale bar, 200 μm. Enlarged images are shown in the lower panels. Scale bar, 50 μm. FIG. 13C shows representative immunostaining of human HNSCC with low BMI1 expression and corresponding high expression of CD8, CCL5, and CXCL10. Scale bar, 200 μm. Enlarged images are shown in the lower panels. Scale bar, 50 μm.

FIGS. 14A-14I show BMI1 inhibition activates cGAS-STING-IRF3 signaling by inducing DNA damage and cytosolic DNA accumulation. FIG. 14A shows immunofluorescent staining of pH2A.X (green) in SCC23 and SCC1 cells treated with PTC209 or shBMI1 and their quantifications. Nuclei were stained with DAPI (blue). Means±SD are from three independent experiments. Scale bar, 50 μm. **p<0.01 by unpaired Student's t test. FIG. 14B shows Western blot analysis of pH2A.X in SCC23 and SCC1 treated with PTC209 and shBMI1 treatment. FIG. 14C shows representative images and quantification of DNA Comet assays in SCC23 and SCC1 cells treated with PTC209 or shBmi1. More than 200 cells were analyzed per group. Means±SD are shown. Scale bar, 100 μm. **p<0.01 by unpaired Student's t test. FIG. 14D presents confocal images showing cytosolic DNA accumulation in SCC1 cells induced by PTC209 or shBMI1. Double strand DNA (dsDNA) was stained by Picogreen (Green). Mitochondria and nuclei were respectively stained with Mito-tracker (Red) and DAPI (Blue). White arrows indicate cytosolic dsDNA. Scale bar, 10 μm. FIG. 14E shows quantification of cytosolic dsDNA accumulation in SCC1 cells induced by PTC209 or shBMI1. More than 100 cells were analyzed per group. Means±SD are shown. **p<0.01 by unpaired Student's t test. FIG. 14F shows induction of phosphorylation of STING (S366), TBK1 (S172) and IRF3 (S396) in SCC1 cells by PTC209 or shBMI1 treatment. FIG. 14G shows qRT-PCR measurement of IFNβ mRNA expression in SCC1 cells treated with PTC209 or shBMI1. Means±SD are shown. **p<0.01 by unpaired Student's t test. FIG. 14H shows immunofluorescent staining of pIRF3 (green) in 4NQO-induced HNSCC by PTC209 or BMI1 knockout and their quantifications. Nuclei were stained with DAPI (blue). Scale bar, 10 μm. Values are means±SD, n=8, **p<0.01 by unpaired Student's t test. FIG. 14I shows a model depicting how BMI1 inhibition eliminates CSCs and activates tumor cell-intrinsic immunity to enable PD1 blockade, thereby inhibiting HNSCC growth and metastasis, and preventing tumor relapse.

FIGS. 15A-15I show inhibition of BMI1 by PTC209 overcomes melanoma resistance to anti-PD1 by recruiting CD8+ T cells. FIG. 15A shows Western blot analysis of BMI1, EZH2 and H2AK119ub (H2AUb) in B16 cells treated by PTC209. GAPDH, H2A, and H3 are internal controls. FIG. 15B shows qRT-PCR measurement of IFNβ, CCL5, CXCL9, CXCL10, and CXCL11 mRNA expression in B16 cells treated with PTC209. For each pair of bars, left is DMSO and right is PTC209. Means±SD are shown. **p<0.01 by unpaired Student's t test. FIG. 15C shows images of B16 xenografted tumors in immunocompetent mice with different treatment as indicated. Scale bar, 2 cm. FIG. 15D shows anti-PD1 plus PTC209 inhibited B16 melanoma growth in mice. Values are means±SD. n=6, *p<0.05 and **p<0.01 by one-way ANOVA. FIG. 15E shows B16 tumor weights after treatment. Values are means±SD. n=6, *p<0.05 and **p<0.01 by one-way ANOVA. FIG. 15F shows representative immunofluorescent staining for CD8 (red) and S100 (green) of B16 tumors after treatment. Nuclei were visualized by DAPI (blue). Scale bar, 10 μm. FIG. 15G shows quantifications of percentage of CD8⁺ T cells after treatment. Values are mean±SD (n=6). *p<0.05 and **p<0.01 by one-way ANOVA. FIG. 15H shows representative immunofluorescent staining for CD8⁺ T cells and GzmB⁺ T cells. The upper, middle and lower panels respectively show the staining of GzmB (green), CD8 (red), and CD8 co-localization with GzmB. Nuclei were visualized by DAPI (blue). Scale bar, 10 μm. FIG. 15I shows quantifications of percentage of GzmB⁺ CD8+ T cells in B16 melanomas after treatment. Values are mean±SD (n=6). **p<0.01 by one-way ANOVA.

FIGS. 16A-16D show BMI1 inhibition does not affect the expression of ATM, DNA-PKcs, and MRE11 and TAM779 does not affect CSCs in HNSCC. FIG. 16A shows qRT-PCR measurement of ATM, DNA-PKcs, and MRE11 mRNA expression in SCC23 cells treated with PTC209. Values are mean±SD. ns, not significant by unpaired Student's t test. For each pair of bars, left is DMSO and right is PTC209. FIG. 16B shows qRT-PCR measurement of ATM, DNA-PKcs, and MRE11 mRNA expression in SCC23 cells treated with shBMI1. For each pair of bars, left is shCtrl and right is shBMI1. Values are mean±SD. ns, not significant by unpaired Student's t test. FIG. 16C shows representative images of BMI1⁺ tumor cells in HNSCC after treatment. BMI1 staining intensity was scored as: 1=weak; 2=moderate; 3=strong, and only tumor cells with strong BMI1 staining were counted as CSCs. Black arrows indicate BMI1⁺ CSCs. Scale bar, 10 μm. FIG. 16D shows quantification of the percentage of BMI1⁺ CSCs in HNSCC after treatment. Values are mean±SD. n=8, *p<0.05; **p<0.01 by one-way ANOVA.

FIGS. 17A-17B show PTC209 treatment does not significantly change the percentage of neutrophils and MDSCs (Gr1⁺CD11b⁺ cells) in HNSCC. FIG. 17A shows immunofluorescent staining of neutrophil (red) and PCK (green) in HNSCC and their quantifications. Nuclei were stained with DAPI (blue). Values are mean±SD are shown (n=8). Scale bar, 10 μm. ns, not significant by unpaired Student's t test. FIG. 17B shows immunofluorescent staining of Gr1 (red) and CD11b (green) in HNSCC and their quantifications. Nuclei were stained with DAPI (blue). White arrows indicate MDSCs (Gr1⁺CD11b⁺ cells). Values are mean±SD (n=8). Scale bar, 10 μm. ns, not significant by unpaired Student's t test.

FIGS. 18A-18B show Cisplatin induces Bmi1 expression in Tomato⁺ CSCs. FIG. 18A shows representative staining for Bmi1 (green) and Tomato⁺ CSCs (red) in HNSCC. Nuclei were stained with DAPI (blue). White arrows indicate Bmi1 staining in Tomato⁺ cells (upper panels). White dashed lines demark tumor-stromal junction (middle and lower panels). Scale bar, 10 μm. FIG. 18B shows quantifications of mean fluorescence intensity (MFI) of BMI1 staining in Tomato⁺ cells in HNSCC. Values are mean±SD. n=8, **p<0.01 by one-way ANOVA.

FIGS. 19A-19B show BMI1 inhibition does not change the expression of JUN, FOSL1, MMP3, and MMP9 significantly. FIG. 19A shows qRT-PCR measurement of JUN, FOSL1, MMP3, and MMP9 mRNA expression in SCC23 cells treated with PTC209. For each pair of bars, left is DMSO and right is PTC209. Values are mean±SD. ns, not significant by unpaired Student's t test. FIG. 19B shows qRT-PCR measurement of JUN, FOSL1, MMP3, and MMP9 mRNA expression in SCC23 cells treated with shBMI1. For each pair of bars, left is shCtrl and right is shBMI1. Values are mean±SD. ns, not significant by unpaired Student's t test.

### DETAILED DESCRIPTION OF THE INVENTION

The present disclosure shows that combination treatment of anti-PD1 and cisplatin significantly enriched BMI1⁺ cancer stem cells (CSCs) in head and neck squamous cell carcinoma (HNSCC) by in vivo lineage tracing while inhibiting HNSCC growth in a mouse model of HNSCC, suggesting that BMI1⁺ CSCs formed a fundamental basis for HNSCC relapse. In contrast, pharmacological and genetic inhibition of BMI1 eliminated BMI1⁺ CSCs and enabled PD1 blockade therapy, resulting in potent inhibition of metastatic HNSCC and prevention of HNSCC relapses. Unexpectedly, BMI1 inhibition strongly induced tumor cell-intrinsic immune responses by recruiting and activating CD8+ T cells in addition to eliminating BMI1⁺ CSCs. Mechanistically, BMI1 inhibition induced CD8+ T cell-recruiting chemokines by two interrelated mechanisms: 1) stimulating the cGAS-STING signaling to activate IRF3-mediated transcription and 2) erasing repressive H2A ubiquitination on their promoters. Taken together, these results indicate that in addition to purging CSCs, targeting BMI1 would enable immune checkpoint blockade to inhibit metastatic tumor growth and prevent tumor relapse by activating cell-intrinsic immunity.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates. The compositions described herein can be used to treat any suitable mammal, including primates, such as monkeys and humans, or rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly.

Effective doses of the compositions of the present invention, for treatment of conditions or diseases vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, including cancer, will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test bioassays or systems.

Moreover, suitable doses may also be influenced by permissible daily exposure limits of any compound included in a formulation or method as described herein. Such limits are readily available, including, for example, from industry guidance recommendations provided periodically from the U.S. Food and Drug Administration, and the evaluation of these limits are within the knowledge and understanding of one of ordinary skill in the art.

The composition of the invention may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

As used herein, the term “administering” refers to bringing in contact with a compound of the present invention. Administration can be accomplished to cells or tissue cultures, or to living organisms, for example humans. In one embodiment, the present invention encompasses administering the compositions of the present invention to a human subject.

In one embodiment, any of the therapeutic or prophylactic drugs or compositions described herein may be administered simultaneously. In another embodiment, they may be administered at different time point than one another. In one embodiment, they may be administered within a few minutes of one another. In another embodiment, they may be administered within a few hours of one another. In another embodiment, they may be administered within 1 hour of one another. In another embodiment, they may be administered within 2 hours of one another. In another embodiment, they may be administered within 5 hours of one another. In another embodiment, they may be administered within 12 of one another. In another embodiment, they may be administered within 24 hours of one another.

In one embodiment, any of the therapeutic or prophylactic drugs or compositions described herein may be administered at the same site of administration. In another embodiment, they may be administered at different sites of administration.

In one embodiment, the present disclosure provides a method of treating cancer in a patient in need thereof, comprising the steps of (i) administering to the patient an agent that blocks signaling through programmed cell death protein 1 (PD-1); and (ii) administering to the patient an agent that reduces expression or function of B-cell-specific Moloney murine leukemia virus integration site 1 (BMI-1). In one embodiment, the agent in step (i) is administered to the patient before the agent in step (ii) is administered to the patient. In another embodiment, the agent in step (i) is administered to the patient after the agent in step (ii) is administered to the patient. In yet another embodiment, the agent in step (i) is administered to the patient concurrently with the agent in step (ii).

Representative examples of cancers that can be treated by the above method include, but are not limited to, carcinoma, sarcoma, lymphoma, leukemia, germ cell tumor, blastoma, chondrosarcoma, Ewing's sarcoma, malignant fibrous histiocytoma of bone, osteosarcoma, rhabdomyosarcoma, heart cancer, brain cancer, astrocytoma, glioma, medulloblastoma, neuroblastoma, breast cancer, medullary carcinoma, adrenocortical carcinoma, thyroid cancer, Merkel cell carcinoma, eye cancer, gastrointestinal cancer, colon cancer, gallbladder cancer, gastric (stomach) cancer, gastrointestinal carcinoid tumor, hepatocellular cancer, pancreatic cancer, rectal cancer, bladder cancer, cervical cancer, endometrial cancer, ovarian cancer, renal cell carcinoma, prostate cancer, testicular cancer, urethral cancer, uterine sarcoma, vaginal cancer, head cancer, neck cancer, nasopharyngeal carcinoma, hematopoietic cancer, non-Hodgkin lymphoma, skin cancer, basal-cell carcinoma, melanoma, small cell lung cancer, non-small cell lung cancer, or any combination thereof.

In one embodiment, the above method can reduce cancer metastasis in the patient. In another embodiment, the above method reduces the number of BMI-1+ cancer stem cells in the patient.

In one embodiment, the agent that blocks signaling through programmed cell death protein 1 (PD-1) is an anti-PD-1 antibody. The PD-1 pathway has received considerable attention due to its role in eliciting immune checkpoint response of T cells, resulting in tumor cells capable of evading immune surveillance and being highly refractory to conventional chemotherapy. Application of anti-PD-1/PD-L1 antibodies as checkpoint inhibitors is rapidly becoming a promising therapeutic approach in treating tumors. The present disclosure encompasses anti-PD-1 antibodies that are currently in use, in development, or those that will be developed in the future. Examples of anti-PD-1 antibodies include, but are not limited to, nivolumab (OPDIVO), pembrolizumab (KEYTRUDA), cemiplimab (LIB TAYO), avelumab (BAVENCIO), durvalumab (IMFINZI), and atezolizumab (TECENTRIQ).

In another embodiment, the agent that blocks signaling through PD-1 is an antibody that binds a ligand of PD-1. The present disclosure encompasses ligands of PD-1 that are currently known or those that will be discovered in the future. The present disclosure also encompasses anti-PD-1 ligand antibodies that are currently known or those that will be developed in the future. Examples of ligands of PD-1 include, but are not limited to, PD-L1 and PD-L2.

In one embodiment, the agent that reduces expression or function of BMI-1 is a BMI-1 inhibitor. The present disclosure encompasses BMI-1 inhibitors that are currently in use, in development, or those that will be developed in the future. Examples of BMI-1 inhibitors include, but are not limited to, PTC209, PTC596, PRT4165, PTC-209 HBr, and PTC-028. PTC-209 is a potent and selective BMI-1 inhibitor with IC₅₀ of 0.5 μM in HEK293T cell line, and results in irreversible reduction of cancer-initiating cells. PTC596 is a second-generation BMI-1 inhibitor that accelerates BMI-1 degradation. Upon oral administration, PTC596 targets BMI1 expressed by both tumor cells and cancer stem cells, and induces hyper-phosphorylation of BMI1, leading to its degradation. IC₅₀ values for PTC596 at 72 hours ranged from 68 to 340 nM in mantle cell lymphoma (MCL) cell lines. PRT4165 is a BMI-1 inhibitor with an IC₅₀ of 3.9 μM in cell-free assay. PTC-209 HBr is the hydrobromide salt of PTC-209. PTC-028 is an orally bioavailable compound that decreases BMI-1 levels by posttranslational modification.

In one embodiment, the BMI-1 inhibitor is PTC209, N-(2,6-dibromo-4-methoxyphenyl)-4-(2-methylimidazo[1,2-a]pyrimidin-3-yl)thiazol-2-amine, CAS number 315704-66-6, as described in Kreso A., Van Galen P., Pedley N. M., Lima-Fernandes E., Frelin C., Davis T., et al. (2014), Self-renewal as a therapeutic target in human colorectal cancer. Nat. Med. 20, 29-36, and has the following structure:

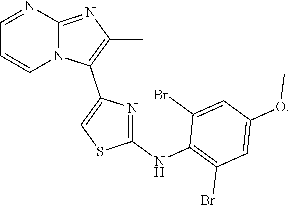 [SMILES: COc1cc(Br)c(Nc2nc(-c3c(C)nc4ncccn34)cs2)c(Br)c1]

In one embodiment, the BMI-1 inhibitor is PTC596, 5-fluoro-2-(6-fluoro-2-methyl-1H-benzimidazol-1-yl)-N-[4-(trifluoromethyl)phenyl]pyrimidine-4,6-diamine, also called unesbulin, CAS number 1610964-64-1, as described by Nishida Y, Maeda A, Kim M J, Cao L, Kubota Y, Ishizawa J, AlRawi A, Kato Y, Iwama A, Fujisawa M, Matsue K, Weetall M, Dumble M, Andreeff M, Davis T W, Branstrom A, Kimura S, Kojima K. The novel BMI-1 inhibitor PTC596 downregulates MCL-1 and induces p53-independent mitochondrial apoptosis in acute myeloid leukemia progenitor cells. Blood Cancer J. 2017 Feb. 17; 7(2):e527, and by Maeda A, Nishida Y, Weetall M, Cao L, Branstrom A, Ishizawa J, Nii T, Schober W D, Abe Y, Matsue K, Yoshimura M, Kimura S, Kojima K. Targeting of BMI-1 expression by the novel small molecule PTC596 in mantle cell lymphoma. Oncotarget. 2018 Jun. 19; 9(47):28547-28560, and has the following structure:

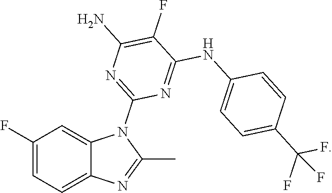 [SMILES: Cc1nc2ccc(F)cc2n1-c1nc(N)c(F)c(Nc2ccc(C(F)(F)F)cc2)n1]

In one embodiment, the BMI-1 inhibitor is PTC-209 hydrobromide, N-(2,6-dibromo methoxyphenyl)-4-(2-methylimidazo[1,2-a]pyrimidin-3-yl)-1,3-thiazol-2-amine hydrobromide, CAS number 1217022-63-3, is the hydrobromide salt of PTC209 described above, and has the following structure:

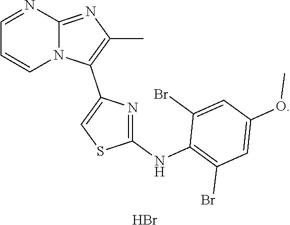 [SMILES: COc1cc(Br)c(Nc2nc(-c3c(C)nc4ncccn34)cs2)c(Br)c1 ; Br]

In one embodiment, the BMI-1 inhibitor is PTC-028, 6-(5,6-difluoro-2-methyl-1H-benzo[d]imidazol-1-yl)-N-(4-(trifluoromethyl)phenyl)pyrazin-2-amine, CAS number 1782970-28-8, such as described in Bolomsky A, Muller J, Stangelberger K, Lejeune M, Duray E, Breid H, Vrancken L, Pfeiffer C, Hübl W, Willheim M, Weetall M, Branstrom A, Zojer N, Caers J, Ludwig H. The anti-mitotic agents PTC-028 and PTC596 display potent activity in pre-clinical models of multiple myeloma but challenge the role of BMI-1 as an essential tumour gene. Br J Haematol. 2020 September; 190(6):877-890, and has the following structure:

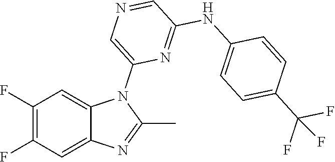 [SMILES: Cc1nc2cc(F)c(F)cc2n1-c1cncc(Nc2ccc(C(F)(F)F)cc2)n1]

In one embodiment, the BMI-1 inhibitor PRT4165, 2-(3-pyridinylmethylene)-1H-indene-1,3(2H)-dione or 2-(3-pyridylmethylene)-1,3-indandione, CAS number 31083-55-3, such as described in Ismail I H, McDonald D, Strickfaden H, Xu Z, Hendzel M J. A small molecule inhibitor of polycomb repressive complex 1 inhibits ubiquitin signaling at DNA double-strand breaks. J Biol Chem. 2013 Sep. 13; 288(37):26944-54, and has the following structure:

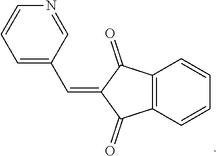 [SMILES: O=C1C(=Cc2cccnc2)C(=O)c2ccccc21]

In one embodiment, the agent that reduces expression or function of BMI-1 is a small interfering RNA (siRNA). One of ordinary skill in the art would readily construct and use siRNA against a target such as BMI-1.

In another embodiment, there is provided a method of increasing anti-tumor T cell activities in a patient having a cancer, comprising the steps of (i) administering to the patient an agent that blocks signaling through programmed cell death protein 1 (PD-1); and (ii) administering to the patient an agent that reduces expression or function of B-cell-specific Moloney murine leukemia virus integration site 1 (BMI-1). The agent in step (i) can be administered to the patient before, after, or at the same time as the agent in step (ii) is administered to the patient. Examples of cancer that can be treated have been discussed above. In one embodiment, the anti-tumor T cell activities are mediated by CD8+ T cells.

In one embodiment, the agent that blocks signaling through PD-1 is anti-PD-1 antibody or antibody that binds a ligand of PD-1. Examples of anti-PD-1 antibodies or ligands of PD-1 have been discussed above.

In one embodiment, the agent that reduces expression or function of BMI-1 is a BMI-1 inhibitor or small interfering RNA (siRNA). Examples of BMI-1 inhibitors have been discussed above.

In another embodiment, there is provided a method of reducing the number of cancer stem cells in a cancer patient in need thereof, comprising the steps of (i) administering to the patient an agent that blocks signaling through programmed cell death protein 1 (PD-1); and (ii) administering to the patient an agent that reduces expression or function of B-cell-specific Moloney murine leukemia virus integration site 1 (BMI-1). The agent in step (i) can be administered to the patient before, after, or at the same time as the agent in step (ii) is administered to the patient. Examples of cancer that can be treated have been discussed above. In one embodiment, the cancer stem cells are BMI-1+.

In one embodiment, the agent that blocks signaling through PD-1 is anti-PD-1 antibody or antibody that binds a ligand of PD-1. Examples of anti-PD-1 antibodies or ligands of PD-1 have been discussed above.

In one embodiment, the agent that reduces expression or function of BMI-1 is a BMI-1 inhibitor or small interfering RNA (siRNA). Examples of BMI-1 inhibitors have been discussed above.

In another embodiment, there is provided a composition for treating cancer in a patient, comprising (i) a composition of an agent that blocks signaling through programmed cell death protein 1 (PD-1); and (ii) a composition of an agent that reduces expression or function of B-cell-specific Moloney murine leukemia virus integration site 1 (BMI-1). In one embodiment, the agent that blocks signaling through PD-1 is anti-PD-1 antibody or antibody that binds a ligand of PD-1. Examples of anti-PD-1 antibodies include, but are not limited to, nivolumab (OPDIVO), pembrolizumab (KEYTRUDA), cemiplimab (LIB TAYO), avelumab (BAVENCIO), durvalumab (IMFINZI), and atezolizumab (TECENTRIQ). In another embodiment, the antibodies bind to ligands of PD-1 such as PD-L1 or PD-L2. In another embodiment, the agent that reduces expression or function of BMI-1 is a BMI-1 inhibitor or small interfering RNA (siRNA). Examples of BMI-1 inhibitors include, but are not limited to, PTC209, PTC596, PRT4165, PTC-209 HBr, or PTC-028. One of ordinary skill in the art could readily employ standard techniques to determine desirable doses for the agents in this composition for cancer treatment. For example, current or previous clinical trials or clinical uses of BMI-1 inhibitors and anti-PD-1 antibodies would provide reference data for determining the suitable doses. In one embodiment, BMI-1 inhibitors (e.g. PTC596) can be administered at 200 mg orally twice weekly. In another embodiment, anti-PD-1 antibodies such as nivolumab can be administered at 240 mg IV over 30 minutes every 2 weeks, or 480 mg IV over 30 minutes every 4 weeks, or 1 mg/kg IV over 30 minutes every 3 weeks. Alternatively, anti-PD-1 antibodies such as pembrolizumab can be administered at 200 mg IV over 30 minutes every 3 weeks. In yet another embodiment, anti-PD-1 antibodies such as cemiplimab can be administered at 350 mg IV over 30 minutes every 3 weeks.

In another embodiment, there is provided a therapeutic combination of compositions formulated for treating cancer in a patient, comprising (i) a composition of an agent that blocks signaling through programmed cell death protein 1 (PD-1); and (ii) a composition of an agent that reduces expression or function of B-cell-specific Moloney murine leukemia virus integration site 1 (BMI-1). Examples of agents in the composition of (i) and composition of (ii) have been described above.

In one embodiment, there is provided uses of the above composition or therapeutic combination of composition in treating cancer in a patient. Examples of cancers, as well as agents in the composition of (i) and composition of (ii) have been described above. Examples of methods of administering the composition to the patient have been discussed above. One of ordinary skill in the art would determine the doses of the agents in the compositions by standard techniques generally known in the art. In one embodiment, composition of (i) is administered to the patient before, after, or concurrently as the composition of (ii) is administered to the patient.

The terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “an enzyme” or “at least one enzyme” may include a plurality of enzymes, including mixtures thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

In the description presented herein, each of the steps of the invention and variations thereof are described. This description is not intended to be limiting and changes in the components, sequence of steps, and other variations would be understood to be within the scope of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Example 1

### BMI1 Ablation Eliminates Cancer Stem Cells and Activates Antitumor Immunity

### Materials and Methods

### Mice

Bmi1^{CreER} (JAX:010531) and R26^{tdTomato} (JAX:007908) mouse strains were cross-mated to generate Bmi1^{CreER};R26^{tdTomato}. K14^{CreER} (JAX:005107) and Bmi1^{flox/flox} (JAX: 028974) mouse strains were cross-mated to generate K14^{CreER}; Bmi1^{flox/flox}. All of these above mice were purchased from The Jackson Laboratory and housed under specific-pathogen-free (SPF) conditions in the UCLA animal facility. All mouse experiments were performed per protocols approved by UCLA Animal Research Committee. For induction of HNSCC, six-week-old mice were treated with drinking water containing 50 ug/ml 4NQO (Santa Cruz, Cat #256815) for 16 weeks and then given normal drinking water for tumor formation and lymph node metastasis. For lineage tracing and Bmi1 knock out studies, mice were intraperitoneally injected with tamoxifen (9 mg per 40 g body weight; Sigma-Aldrich, Cat #T5648) to activate Cre.

### Cell Lines

Human HNSCC cell lines SCC23 and SCC1 were from the University of Michigan. B16 cells were from American Type Culture Collection (ATCC, Manassas, Va.). Cells were maintained in DMEM containing 10% FBS and antibiotics (streptomycin and penicillin) at 37° C. in 5% CO2 atmosphere.

### Human HNSCC Samples

The use of human HNSCC samples for immunostaining was approved by the UCLA Institutional Review Board. Human HNSCC paraffin-embedded blocks were obtained from the UCLA Translational Pathological Core Laboratory and processed as described previously (Chen et al., Cell stem cell 20:621-634 (2017); Ding et al., Science signaling 6, ra28.21-13, S20-15 (2013)).

### 4NQO Mouse Model of HNSCC, Treatment And Histology

Cisplatin (Sigma-Aldrich, Cat #479306) was dissolved in saline. PTC209 (MedChem Express, Cat #HY-15888) was dissolved in 14% DMSO, 36% polyethylene glycol 400 (Sigma-Aldrich, Cat #202304) and 50% polypropylene glycol (Sigma-Aldrich Cat #4347). For treatment, tumor-bearing mice were randomly divided into 4 groups and given: 1) control vehicle and antibody InVivoPlus rat IgG2a isotype (BioXcell Cat #BP0089, 200 μg/mouse); 2) anti-PD1 (BioXcell, Cat #BE0146, 200 μg/mouse twice/week); 3) cisplatin (5 mg/kg body weight once a week) or PTC209 (60 mg/kg body weight twice/week); and 4) anti-PD1 plus cisplatin or anti-PD1 plus PTC-209. The cisplatin dose and frequency chosen was the weekly tolerated dose that did not have severe side effects on mice based on previous studies. For depletion of CD8+ T cells, mice were given anti-mouse CD8 (InVivoPlus, BioXcell Cat #BP0061, 100 μg/mouse twice/week). For the inhibition of CXCR3 and CXCR5, mice were given TAK779 (Sigma-Aldrich, Cat #SML0911, 150 μg/mouse twice/week).

Tumor growth and cervical lymph node metastasis were examined as described before (Chen et al., 2017). Briefly, mice were sacrificed, and tongues and cervical lymph nodes were harvested immediately and the lesion surface areas were measured. For histological analysis and immunostaining, longitudinally cut tongues (dorsal/ventral) and intact lymph nodes were fixed overnight in 10% buffered formalin and paraffin-embedded. Tissue blocks were cut into 10-15 sections in 4 μm thickness and stained with hematoxylin and eosin (H&E). The SCC number was counted and areas were measured as described before (Chen et al., 2017). The HNSCC invasiveness was scored based on the following criteria: showing signs of normal or epithelial dysplasia appearance (grade 1); distinct invasion, unclearness of basement membrane, drop and diffuse infiltration into the superficial portion of the muscle layer (grade 2); loss of the basement membrane, extensive invasion into deep muscle layer (grade 3). To assess lymph node metastasis, the sections of cervical lymph nodes were immunostained with anti-PCK antibodies which specifically detected epithelial tumor cells in lymph nodes (Santa Cruz, Cat #sc-8018). The percentage of lymph nodes with metastasis and their metastatic areas were measured.

### Mouse B16 Melanoma Tumor Models

C57/6J mice were injected in the flank subcutaneously with B16 melanoma cells (250,000 cells per site). Tumors were measured every 3 days once palpable (long diameter and short diameter) with a caliper. Tumor volume was determined using the volume formula for an ellipsoid: ½×D×d² where D is the longer diameter and d is the shorter diameter. Mice were sacrificed when tumors reached 1000 mm³ or upon ulceration/bleeding. For treatment, tumor-bearing mice were randomly divided into 4 groups and given the same treatment strategy as 4NQO-induced HNSCC model.

### Immunostaining

Mouse HNSCC and cervical lymph nodes were harvested and cytosections were prepared and processed as previously described (Chen et al., 2017). For immunofluorescent staining, sections were stained with the following primary antibodies: anti-PCK (Abcam Cat #ab9377; 1:200), anti-Ac-casp3 (Cell Signaling Technology, Cat #9661; 1:200), anti-CD8 (Cell Signaling Technology Cat #98941; 1:200), anti-Granzyme-B, (R&D Systems, Cat #AF1865; 1:100), and anti-S100 (Abcam Cat #ab4066; 1:200). The immunocomplexes were detected and visualized using related secondary antibodies conjugated with Cy2 or Cy3 (Jackson ImmunoResearch Laboratories). Sections were then counterstained with 4′6′-diamidino-2-phenilindole (DAPI; Sigma-Aldrich Cat #D9542) and mounted with SlowFade Antifade Reagents (Thermo Fisher Scientific Cat #536937) for imaging and analysis. For the quantification of CD8+, Ac-casp3⁺ and Tomato⁺ cells, the methods described previously (Miao et al., Cell 177:1172-1186 (2019)) were used with some modifications. At least three sections from each HNSCC lesions were immunostained and analyzed. Tumor cells (>150) and CD8+, Ac-casp3+ and Tomato⁺ cells of these tumor cell areas were counted manually in each section. The percentage of CD8+, Ac-casp3⁺ and Tomato⁺ cells were calculated by dividing those cells with tumor cells and averaged from the sections.

For immunohistochemistry of human or murine HNSCC samples, sections were incubated with the following primary antibodies at 4° C. overnight: anti-BMI1 (Cell Signaling Technology, Cat #5856; 1:50), anti-CD8a (Cell Signaling Technology Cat #85336; 1:100), anti-CCL5 (Abcam Cat #ab9679; 1:100), and anti-CXCL10 (Santa Cruz, Cat #sc-101500; 1:100). The sections were then incubated with horseradish perioxidase-labeled polymer for 60 min. The signals were detected with AEC+ chromogen (Dako EnVision System Cat #MP-6401-15) and counterstained with hematoxylin. The intensity of immunostaining was scored as follows: 0, no staining; 1, weak staining; 2, moderate staining; 3, strong staining; and 4, very strong staining as described before (Chen et al., 2017). The Spearman or Pearson correlation coefficient of liner regression was used to determine the correlation between different proteins in human HNSCC samples.

### Flow Cytometry Analysis

PBMCs were isolated from blood using Ficoll-Paque Plus density gradient centrifugation (GE Healthcare Life Sciences, Cat #17-1440). Cervical lymph nodes and spleens were collected and processed into single-cell suspensions through mechanical separation. The isolated or dissociated cells were stained with the specific surface marker antibodies, anti-CD3-FITC (eBioscience, Cat #11-0031), anti-CD4-APC (eBioscience, Cat #17-0041), and anti-CD8-PerCP-Cy5.5 (eBioscience, Cat #45-0081) in PBS with FBS for 30 min at 4° C. Intracellular staining of IFNγ was performed as follows: cells were stimulated with PMA and ionomysin cocktail (eBioscience, Cat #00-4970) for 5 h at 37° C. with 5% CO₂. Cells were washed and stained with surface marker antibodies, then fixed and permeabilized with a fixation/permeabilization kit (BD Bioscience, Cat #554715) and intracellularly stained with anti-IFNγ-PE (eBioscience, Cat #12-7311). For proper compensation of flow cytometry channels, single-stain samples were utilized, and for gating, isotype controls were applied. The stained cells were analyzed on the BD FACS flow cytometer, and data analyzed using Flowjo software.

Cell Culture and BMI1 Knockdown by shRNA

Human SCC23 and SCC1 cells were grown in DMEM containing 10% FBS and antibiotics (streptomycin and penicillin) at 37° C. in a 5% CO₂ atmosphere. To generate lentiviruses, scramble control (shCtrl, Addgene, Cat #1864; see Sarbassov et al Science 2005 Feb. 18; 307(5712):1098-101) and BMI1 specific shRNA lentiviral plasmids (shBmi1, Sigma-Aldrich, Cat #TRCN0000020156, comprising CCGGCCTAATACTTTCCAGATTGATCTCGAGATCAATCTGGAAAGTATTAGGTTTTT, SEQ ID NO:35) were transfected into HEK293T cells with two helper plasmids psPAX2 (Addgene, Cat #12260) and pMD2.G (Addgene, Cat #12259). Viral supernatant was harvested 72 h after transfection and passed through a 0.45 μm filter to remove cell debris and live cells. Collected lentiviruses were used directly to infect cells with the addition of polybrene (Sigma-Aldrich, Cat #H9268), or frozen at −80° C. for later use. Twenty four hours after infection, cells were selected with puromycin (Sigma-Aldrich, Cat #P9620) at 1 μg/ml for 5 days and then expanded before being used for subsequent assays. The knockdown of BMI1 was confirmed by Western blot analysis.

qRT-PCR and ChIP-qPCR

For qRT-PCR, total RNA was prepared using TRIzol reagent (Thermo Fisher Scientific Cat #15596026), and 1 μg of RNA was reversely transcribed with random primer (Thermo Fisher Scientific Cat #48190011), dNTP mix (Thermo Fisher Scientific, Cat #18427013), and M-MuLV Reverse Transcriptase (New England Biolabs, Cat #M0253L). The levels of mRNA were qualitatively measured using a SYBRGreen supermix (Bio-Rad, Cat #1708880). GAPDH was used as an internal control.

ChIP-qPCR assays were performed as previously described (Ding et al, 2013). Briefly, SCC cells were sequentially treated with dimethyl 3,3′-dithiobispropionimidate-HCl (DTBP; Cat #20665, Thermo Fisher Scientific) solution and formaldehyde, and harvested with a cell scraper. The cell pellet was lysed with ChIP lysis buffer and sonicated to generate 200-500 bp DNA fragments with a sonicator. The fragmented chromatins were immunoprecipitated with anti-BMI1 (Cell Signaling Technology, Cat #6964), anti-Ubiquityl-Histone H2A (Lys119) (Cell Signaling Technology, Cat #8240) overnight at 4° C. The precipitated DNA-chromatin products were purified with ChIP DNA clean & concentrator kit (Cat #D5205, Zymo Research) and the DNA levels were quantified by qPCR. Data is presented as the percentage of input DNA. The primer sequences used for qRT-PCR and ChIP-qPCR were listed in Table 1.

### Western Blot and ELISA Assays

Cells were lysed using the radioimmunoprecipitation assay (RIPA) buffer (Sigma-Aldrich, Cat #R0278) added with a cocktail of protease inhibitors (Thermo Fisher Scientific, Cat ##78430) and phosphatase inhibitors (Sigma-Aldrich, Cat #4906845001). Protein extracts were resolved on a 10% or 15% SDS polyacrylamide gel and then transferred to a polyvinylidene difluoride membrane. Membranes were blocked with 5% milk for 1 h and incubated with primary antibodies overnight at 4° C. Primary antibodies used in this study were: anti-phospho-STING (Ser366) (1:1,000; Cell Signaling Technology, Cat #19781), anti-STING (1:1,000; Cell Signaling Technology, Cat #13647), anti-phospho-TBK1 (Ser172) (1:1,000; Cell Signaling Technology, Cat #5483), anti-TBK1 (1:1,000; Cell Signaling Technology, Cat #3504), anti-phospho-IRF3 (Ser396) (1:1,000; Cell Signaling Technology, Cat #29047), anti-IRF3 (1:1,000; Cell Signaling Technology, Cat #4302), anti-BMI1 (1:1,000; Cell Signaling Technology, Cat #6964), anti-Ubiquityl-Histone H2A (Lys119) (1:1000, Cell Signaling Technology, Cat #8240), anti-phospho Histone H2A.X (Ser139) (1:1000; Cell Signaling Technology, Cat #9718), and anti-GAPDH (1:1000; Cell Signaling Technology, Cat #5174), anti-Histone H3 (1:2,000; Cell Signaling Technology, Cat #4499). The signals were detected using the Clarity Western ECL kit (Bio-Rad, Cat #1705060). To measure the protein levels of CCL5, CXCL9, CXCL10 and CXCL11, cells were treated with PTC209 or shBMI1 knockdown for 48 hr. After treatment, supernatants were collected, and the protein levels of CCL5, CXCL9, CXCL10 and CXCL11 were measured with ELISA (R&D Systems, Cat #DRN00B, DCX900, DIP100, DCX110) according to the manufacturer's instructions.

### RNA-Seq And Pathway Enrichment Analysis

Total RNA was isolated from PTC209-treated or shBMI1 knockdown SCC cells using a RNeasy Micro Kit (QIAGEN, Cat #74004). RNA quality was examined using an Agilent 2100 Bioanalyzer. Library preparation using the KAPA RNA-Seq Library Preparation Kits (KAPA Biosystems, Cat #07960140001) was performed at the UCLA sequencing core facility, and RNAs were single-end sequenced on Illumina HiSeq 3000 machines. The online DAVID bioinformatics resources were used to analyze the differentially expressed genes under the category of GOTERM_BP_DIRECT. The heatmap was generated with Heatmap Builder. The raw data was deposited at the Gene Expression Omnibus (GEO) under the subseries entry GSE140433.

Cytosolic dsDNA Staining

Following the treatment, SCC23 and SCC1 cells were incubated with culture media containing PicoGreen (dsDNA stain, 200-fold dilution, Thermo Fisher Scientific, Cat #P11496) and MitoTracker (mitochondrial dsDNA stain, 100 nM, Thermo Fisher Scientific, Cat #M7512). One hour after incubation, cells were fixed with 4% paraformaldehyde for 10 min. Cells were then washed twice with PBS and stained with DAPI (Sigma-Aldrich, Cat #D9542) and mounted with SlowFade Antifade Reagents (Thermo Fisher Scientific, Cat #536937). Staining was imaged and assessed using a Leica SP5X laser scanning confocal microscope.

### Comet Assays

Single cell gel electrophoresis comet assays were performed using the SCGE assay Kit (Enzo Life Sciences, Cat #ADI-900-166). Following the treatment, cells were mixed with low melting point agarose at a volume ratio of 1:50, and 100 μl of aliquots were loaded onto pre-warmed slides. Slides were incubated in pre-chilled lysis solution for 1 h and then in pre-chilled alkaline solution for 1 h. Electrophoresis was run at 22 V in the TBE buffer for 30 min. Comets were stained with CYGREEN dye for 30 min and imaged. 50 individual cells at least per sample were evaluated in duplicates by the CASP Version 1.2.2 analysis tool.

### Statistical Analyses

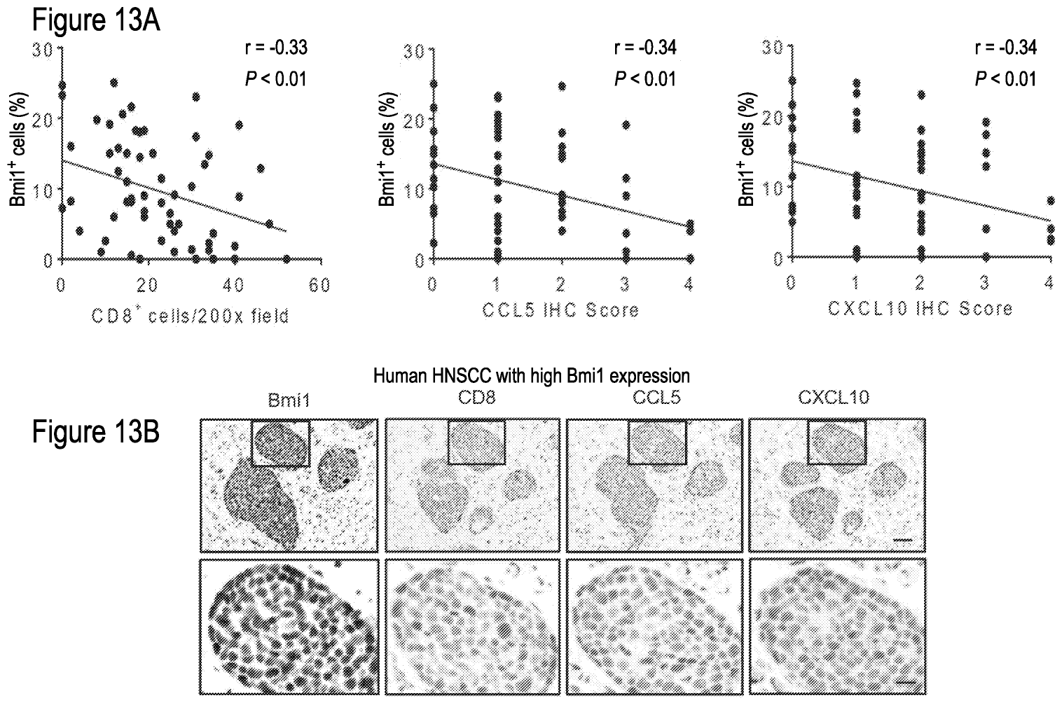

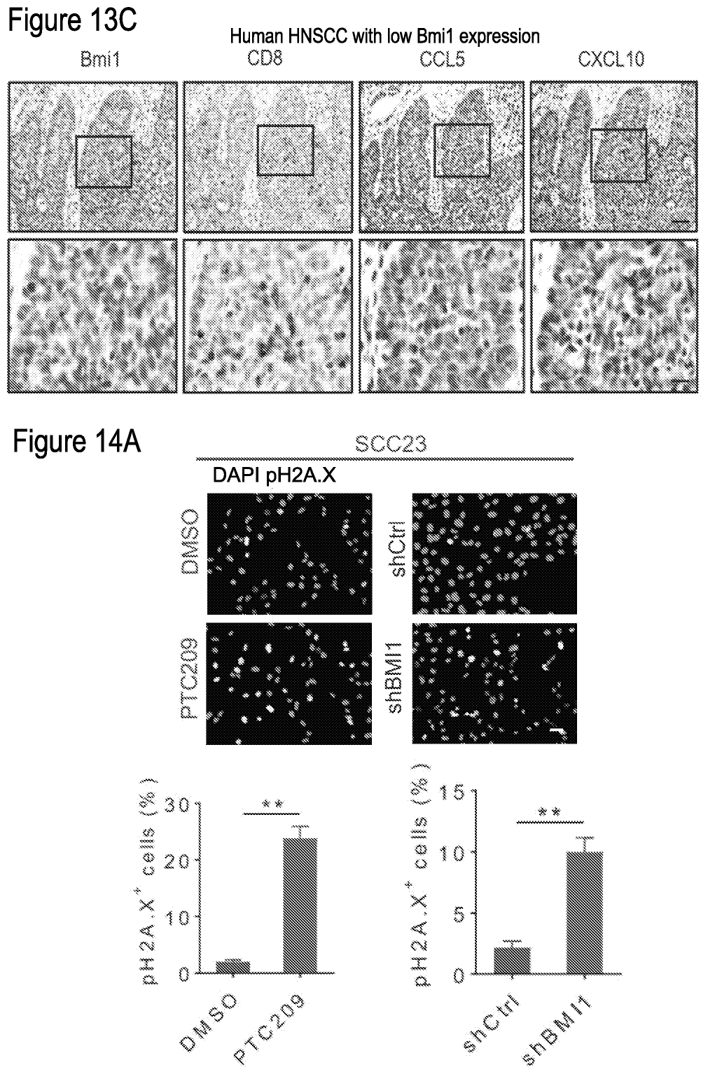

Statistical parameters of the analyses are reported in the Figure Legends. All in vitro experiments were repeated at least twice, and in vivo experiments were repeated at least once. Statistical analyses were performed using GraphPad Prism 6.0 for windows (GraphPad software, Inc.). Statistical parameters of the analyses are reported in the Figure Legends. To compare HNSCC lesion size, number and area in control and knockout mice, the differences were assessed using two-way ANOVA. For comparison of treatment in same stain of mouse, the differences were evaluated by one-way ANOVA followed by the Tukey's HSD post-hoc tests to minimize type I errors. For ANOVA analyses, we utilized Shapiro-Wilk test to validate normal distribution of data and that all data met the assumptions of no significant outliers. A total of 60 human HNSCC samples were used in this study, and the parameters for scores was reported in the Figure legends (FIG. 13A). The Pearson and Spearman correlation coefficient of liner regression was used to assess the correlation between different proteins.

TABLE 1
Sequences for qRT-PCR and ChIP-qPCR primers

| | | |
|---|---|---|
| Target Genes | Forward (5′-3′) | Reverse (5′-3′) |

Primers for qRT-PCR

| | | |
|---|---|---|
| human GAPDH | AACGGGAAGCTTGTCATCAA | TGGACTCCACGACGTACTCA |
| | (SEQ ID NO: 1) | (SEQ ID NO: 2) |
| human CCL5 | CAGTGGCAAGTGCTCCAACC | CCATCCTAGCTCATCTCCAAAGAGT |
| | (SEQ ID NO: 3) | (SEQ ID NO: 4) |
| human CXCL9 | GTGGTGTTCTTTTCCTCTTGGG | ACAGCGACCCTTTCTCACTAC |
| | (SEQ ID NO: 5) | (SEQ ID NO: 6) |
| human CXCL10 | GCAAGCCAATTTTGTCCACG | ACATTTCCTTGCTAACTGCTTTCAG |
| | (SEQ ID NO: 7) | (SEQ ID NO: 8) |
| human CXCL11 | CAGAATTCCACTGCCCAAAGG | GTAAACTCCGATGGTAACCAGCC |
| | (SEQ ID NO: 9) | (SEQ ID NO: 10) |
| human IFNβ | GCCATCAGTCACTTAAACAGC | GAAACTGAAGATCTCCTAGCCT |
| | (SEQ ID NO: 11) | (SEQ ID NO: 12) |
| mouse GAPDH | CTCATGACCACAGTCCATGC | CACATTGGGGGTAGGAACAC |
| | (SEQ ID NO: 13) | (SEQ ID NO: 14) |
| mouse CCL5 | CACCACTCCCTGCTGCTT | ACACTTGGCGGTTCCTTC |
| | (SEQ ID NO: 15) | (SEQ ID NO: 16) |
| mouse CXCL9 | TTTTGGGCATCATCTTCCTGG | GAGGTCTTTGAGGGATTTGTAGTGG |
| | (SEQ ID NO: 17) | (SEQ ID NO: 18) |
| mouse CXCL10 | CTTCTGAAAGGTGACCAGCC | GTCGCACCTCCACATAGCTT |
| | (SEQ ID NO: 19) | (SEQ ID NO: 20) |
| mouse CXCL11 | AACAGGAAGGTCACAGCCATAGC | TTTGTCGCAGCCGTTACTCG |
| | (SEQ ID NO: 21) | (SEQ ID NO: 22) |
| mouse IFNβ | GGTGGAATGAGACTATTGTTG | AGGACATCTCCCACGTC |
| | (SEQ ID NO: 23) | (SEQ ID NO: 24) |
| mouse IFNβ | GGTGGAATGAGACTATTGTTG | AGGACATCTCCCACGTC |
| | (SEQ ID NO: 25) | (SEQ ID NO: 26) |

Primers for ChIP

| | | |
|---|---|---|
| human CCL5 | Forward (−215) | CACCATTGGTGCTTGGTCAAAGAGG |
| | | (SEQ ID NO: 27) |
| | Reverse (+115) | GCAGTAGCAATGAGGATGACAGCGA |
| | | (SEQ ID NO: 28) |
| human CXCL9 | Forward (−219) | TGTGCCAAAGGCTATCAGTG |
| | | (SEQ ID NO: 29) |
| | Reverse (−117) | CAGATCCAAGGGAATTTCTGC |
| | | (SEQ ID NO: 30) |
| human CXCL10 | Forward (−363) | TAGGTTTACCTATAAAGGATGAA |
| | | (SEQ ID NO: 31) |
| | Reverse (−92) | ACTCGAAGGTATTATTTATTGTAG |
| | | (SEQ ID NO: 32) |
| human CXCL11 | Forward (−264) | GGTTTTCACAGTGCTTTCAC |
| | | (SEQ ID NO: 33) |
| | Reverse (−154) | TTTCCCTCTTTGAGTCATGC |
| | | (SEQ ID NO: 34) |

### Results

BMI1⁺ CSCs Enriched after Combination of Cisplatin and Anti-PD1

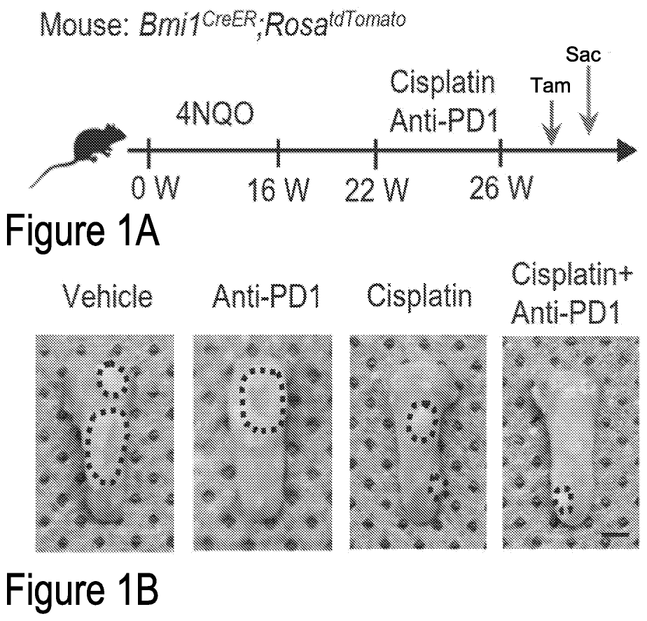

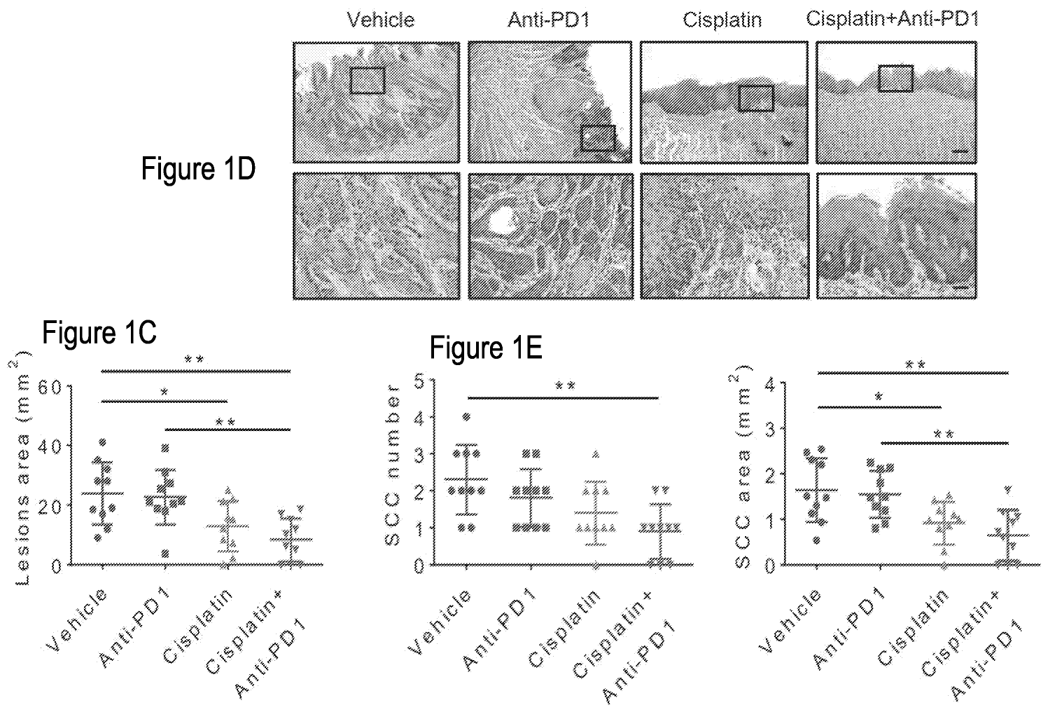

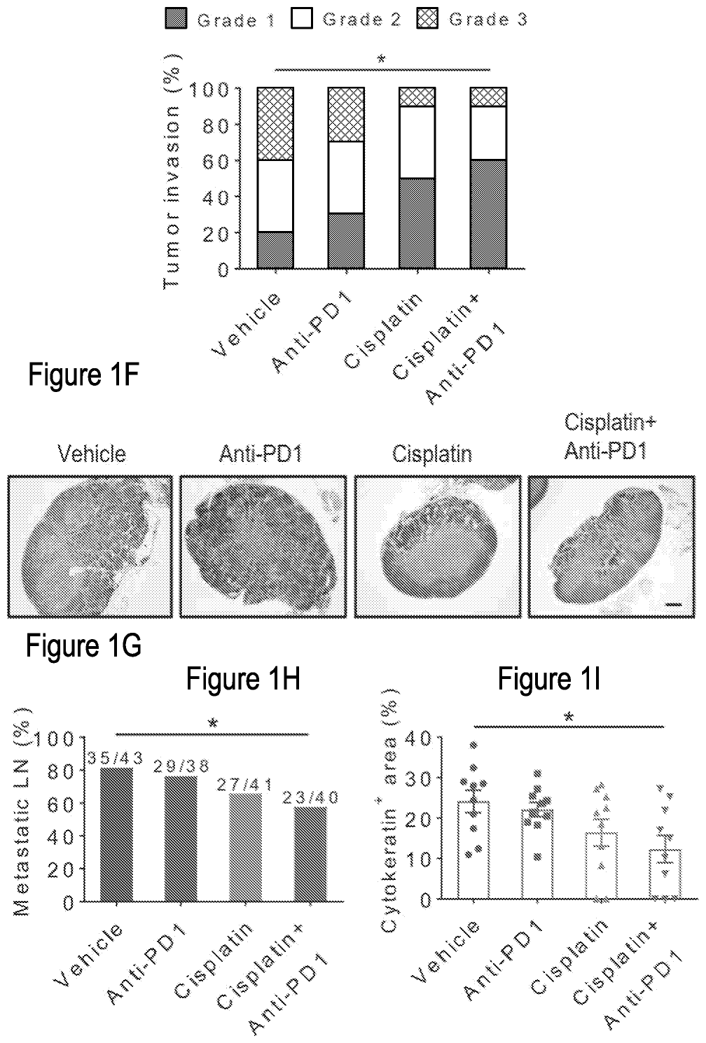

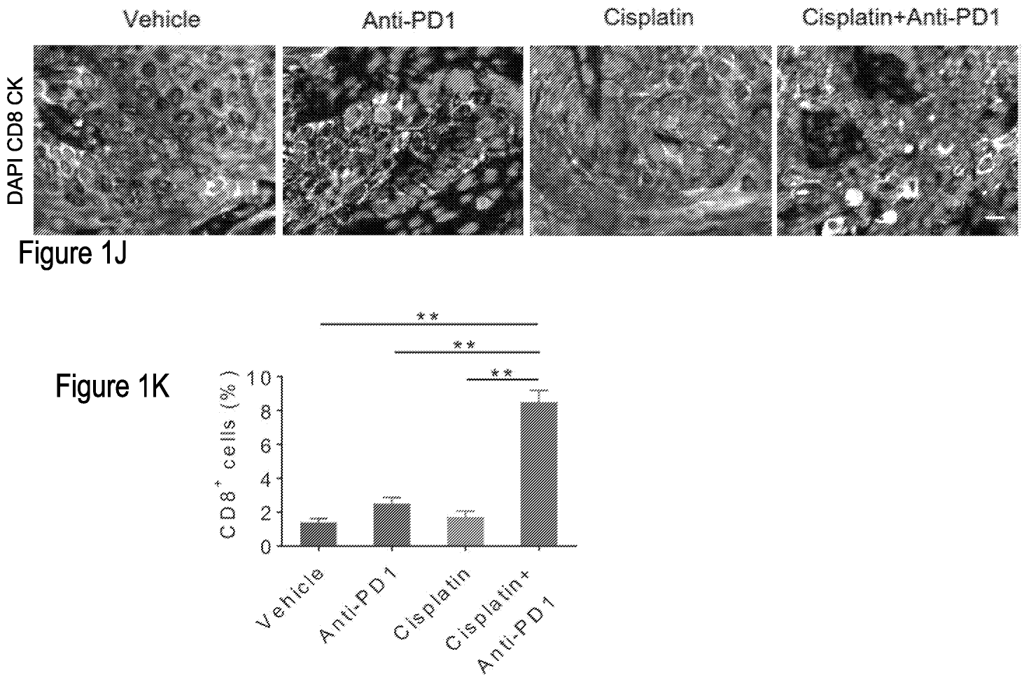

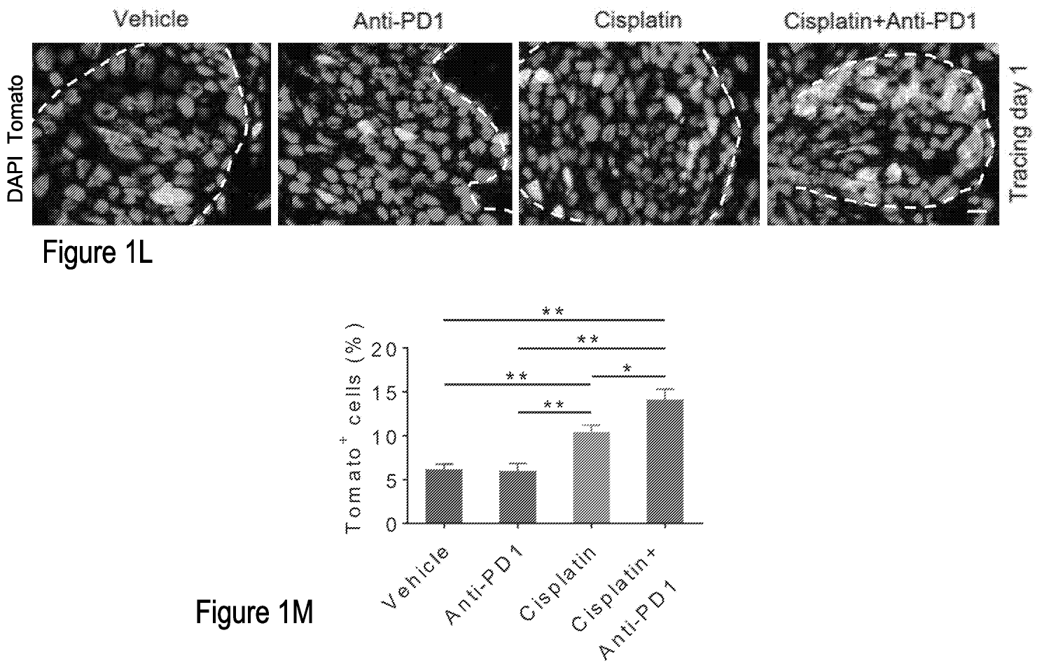

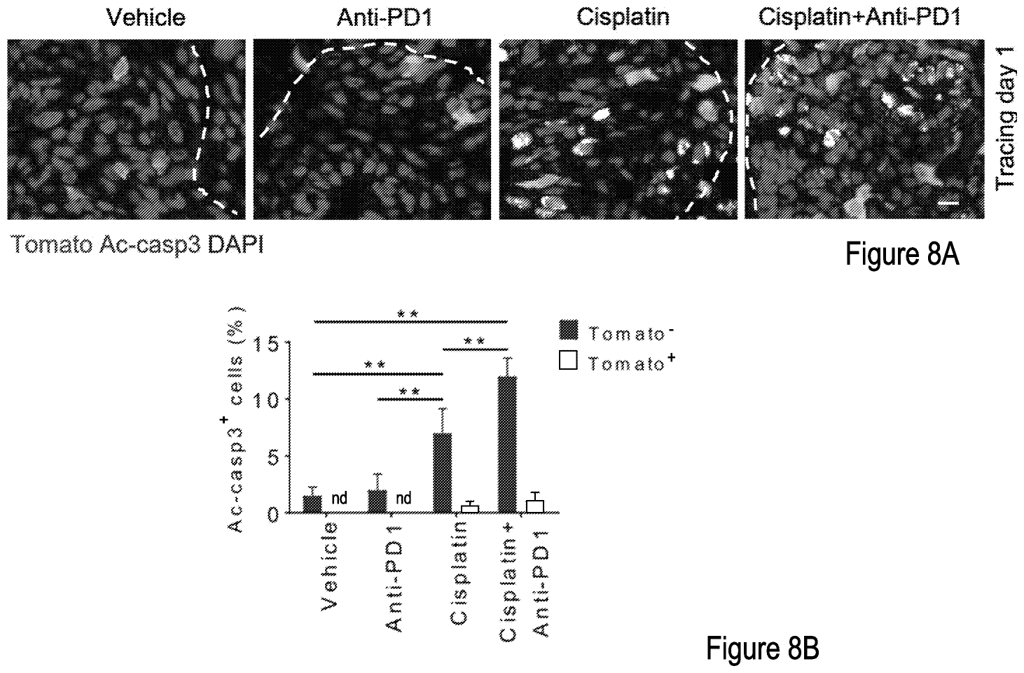

To examine whether anti-PD1 plus cisplatin could eliminate BMI1⁺ CSCs of HNSCC, Bmi1CreER;RosatdTomato mice were treated with 4NQO in their drinking water for 16 weeks and then provided them with normal drinking water. At 22 weeks, the tumor-bearing mice were randomly divided into four groups and treated with cisplatin, anti-PD1, anti-PD1 plus cisplatin, or IgG control for 4 weeks. A single dose of tamoxifen was administered 1 day prior to sacrificing the mice in order to label Tomato⁺ BMI1⁺ CSCs (FIG. 1A). Whereas the treatment of cisplatin alone reduced the lesion surface areas, anti-PD1 did not show inhibition compared with vehicle control. The addition of anti-PD1 to cisplatin did not further enhance inhibitory effects compared with cisplatin alone (FIGS. 1B and 1C). Histological analysis found that cisplatin plus anti-PD1 significantly reduced HNSCC numbers and areas, while such inhibitory effect was not observed by anti-PD1 treatment alone. Although anti-tumor effects between cisplatin alone and anti-PD1 plus cisplatin did not show a statistically significant difference, a trend was observed that anti-PD1 plus cisplatin more effectively reduced SCC numbers (**p<0.01, cisplatin plus anti-PD1 vs. control; not significant, cisplatin vs. control) and areas (**p<0.01, cisplatin plus anti-PD1 vs. control; *p<0.05, cisplatin vs. control) compared with cisplatin treatment alone (FIGS. 1D and 1E). Moreover, anti-PD1 plus cisplatin also significantly reduced the invasiveness of HNSCC (FIG. 1F). Immunostaining with anti-active caspase-3 antibodies (anti-Ac-casp3) demonstrated that apoptotic cells were significantly increased by anti-PD1 plus cisplatin. However, apoptosis was mainly induced in Bmi1⁻ tumor cells rather than BMI1⁺ CSCs (FIGS. 8A and 8B). The cervical lymph nodes are the most common site of HNSCC metastasis which is a key prognostic factor for patients. To accurately detect whether the treatment inhibited HNSCC lymph node metastasis, cervical lymph nodes of mice were immunostained with anti-pan-keratin (PCK) antibodies. Anti-PCK immunostaining revealed that anti-PD1 plus cisplatin, but not cisplatin or anti-PD1 alone, significantly reduced the lymph node metastasis (FIGS. 1G-1I).

Next, the effect of the combination treatment on anti-tumor T cell immunity was investigated. Anti-PD1 or cisplatin alone did not significantly increase CD8+ T cell infiltration in HNSCC. However, immunostaining showed that anti-PD1 plus cisplatin significantly increased CD8+ T cell infiltration (FIGS. 1J and 1K). Since anti-PD1 plus cisplatin efficiently inhibited HNSCC, it is questioned whether the combination could efficiently eliminate BMI1⁺ CSCs by in vivo labeling BMI1⁺ CSCs since BMI1⁺ CSCs were found to play a critical role in HNSCC chemoresistance and relapse (Chen et al., 2017). Anti-PD1 treatment alone did not affect BMI1⁺ CSCs compared with IgG control. Consistent with previous studies, cisplatin significantly enriched BMI1⁺ CSCs in HNSCC. Interestingly, anti-PD1 plus cisplatin further enriched BMI1⁺ CSCs compared with cisplatin alone, probably due to anti-PD1 plus cisplatin capability to efficiently kill non-stem tumor cells (FIGS. 1L and 1M), indicating that BMI1⁺ CSCs also evaded cytotoxic CD8⁺ T cell killing.

### BMI1 Inhibitor Plus Anti-PD1 Eliminates CSCs and Inhibits HNSCC Progression

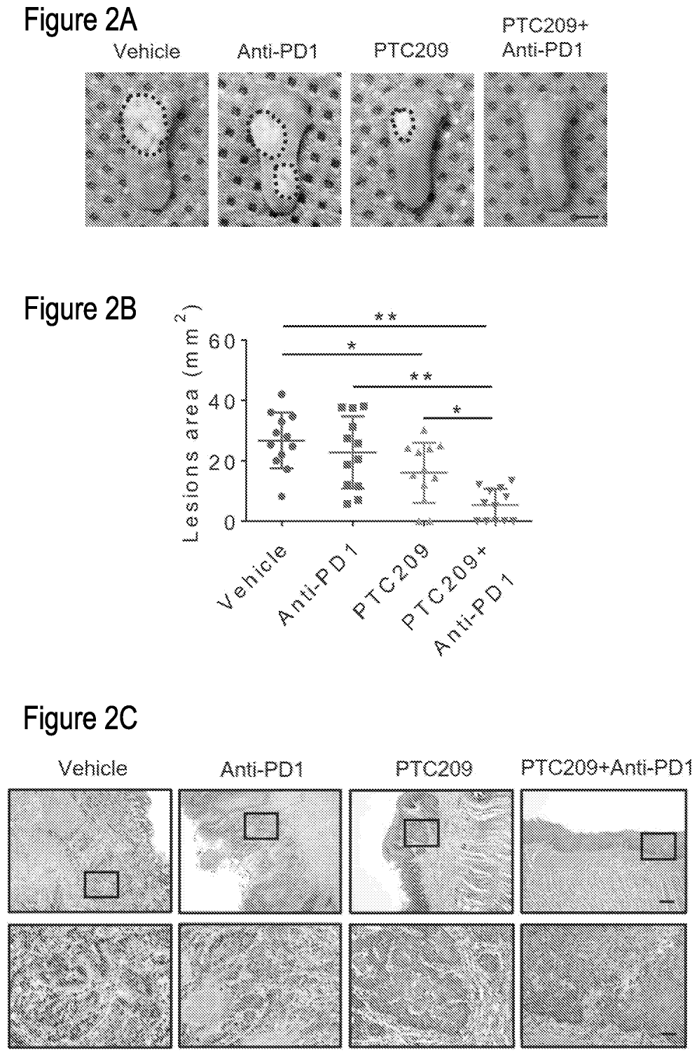

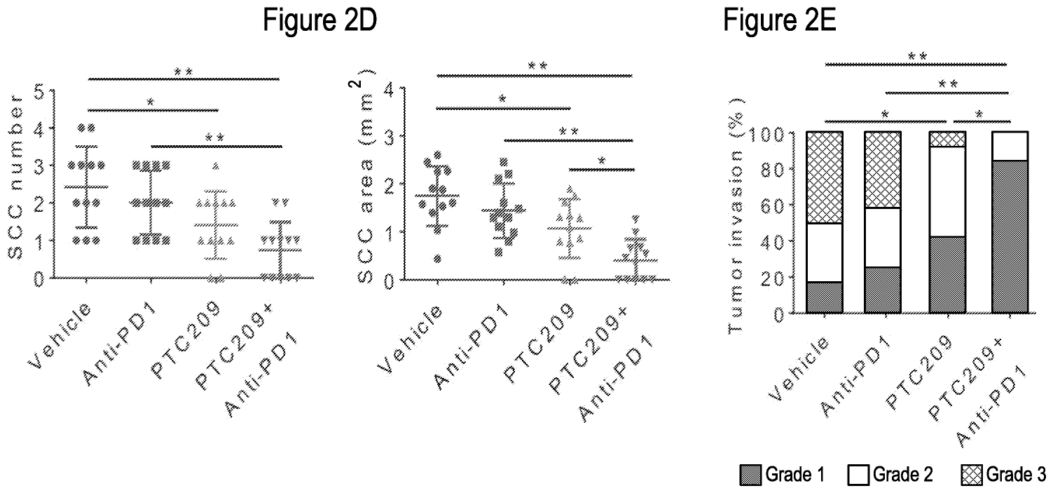

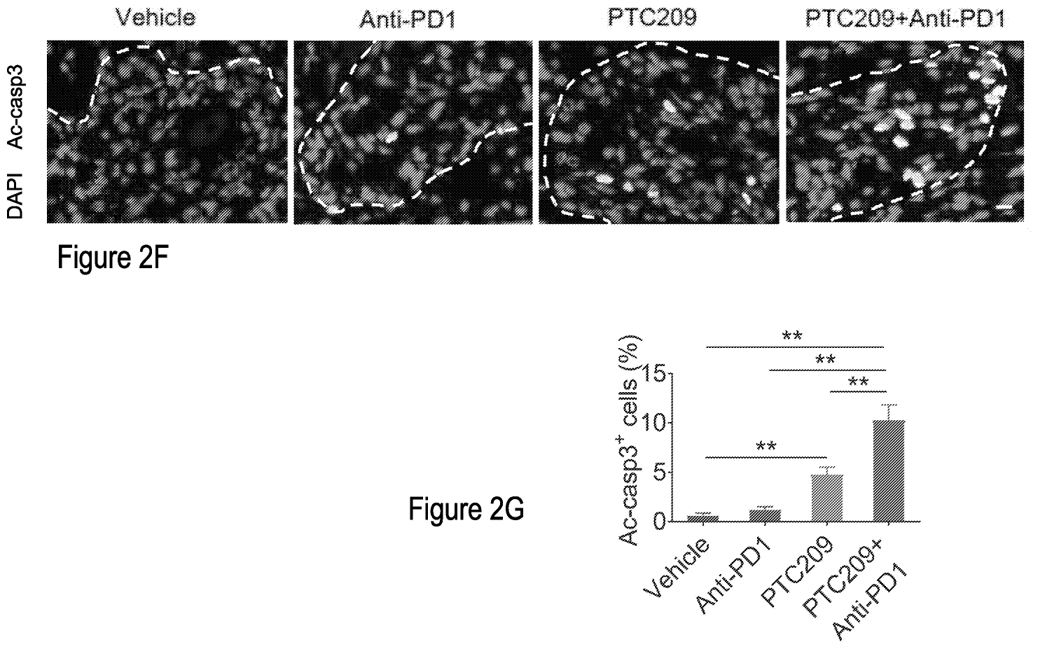

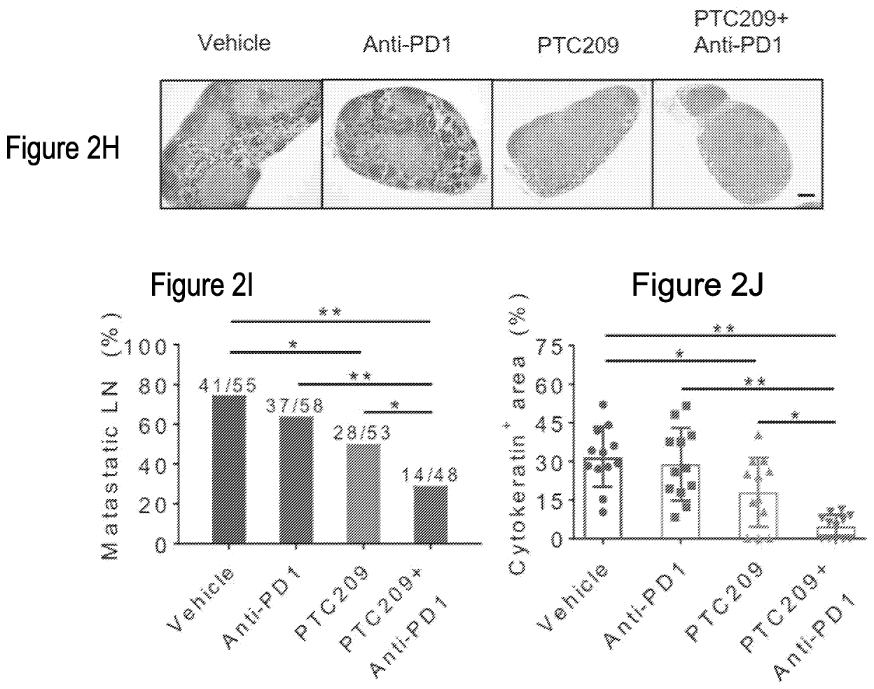

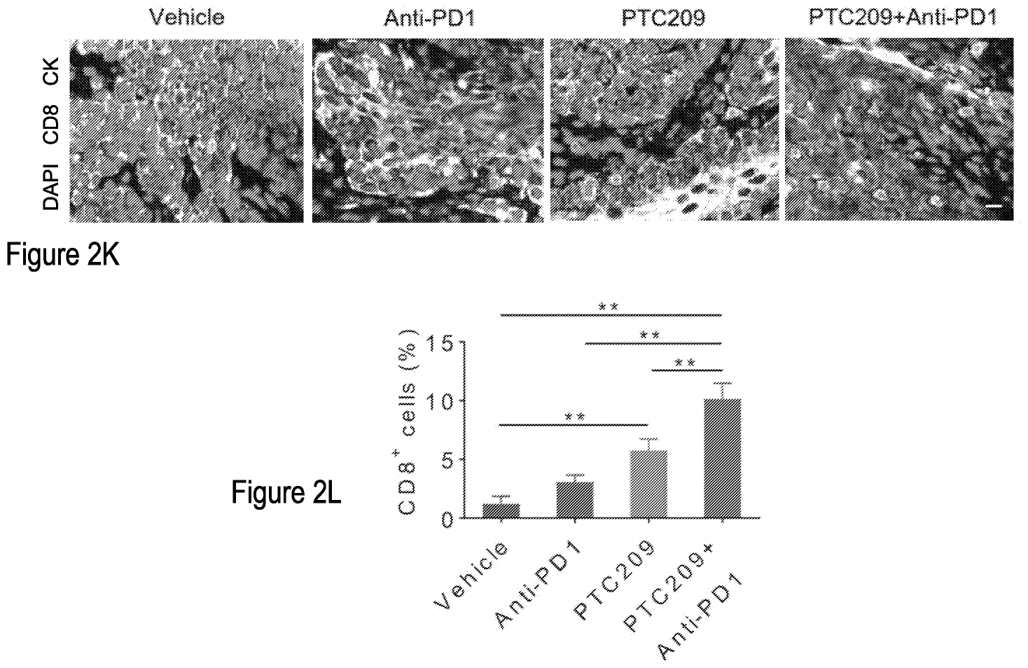

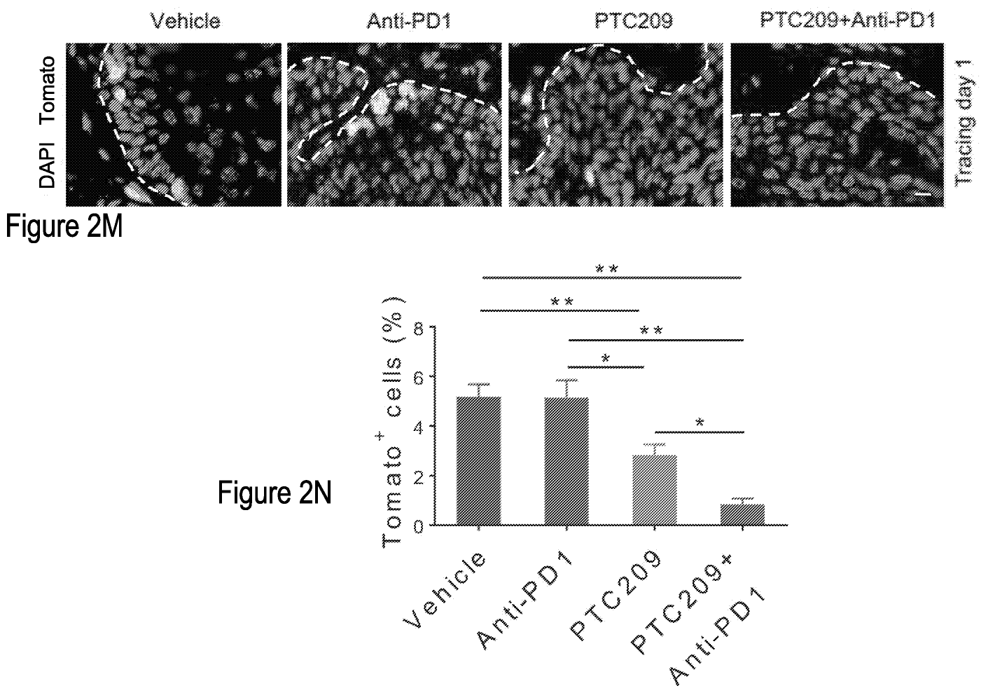

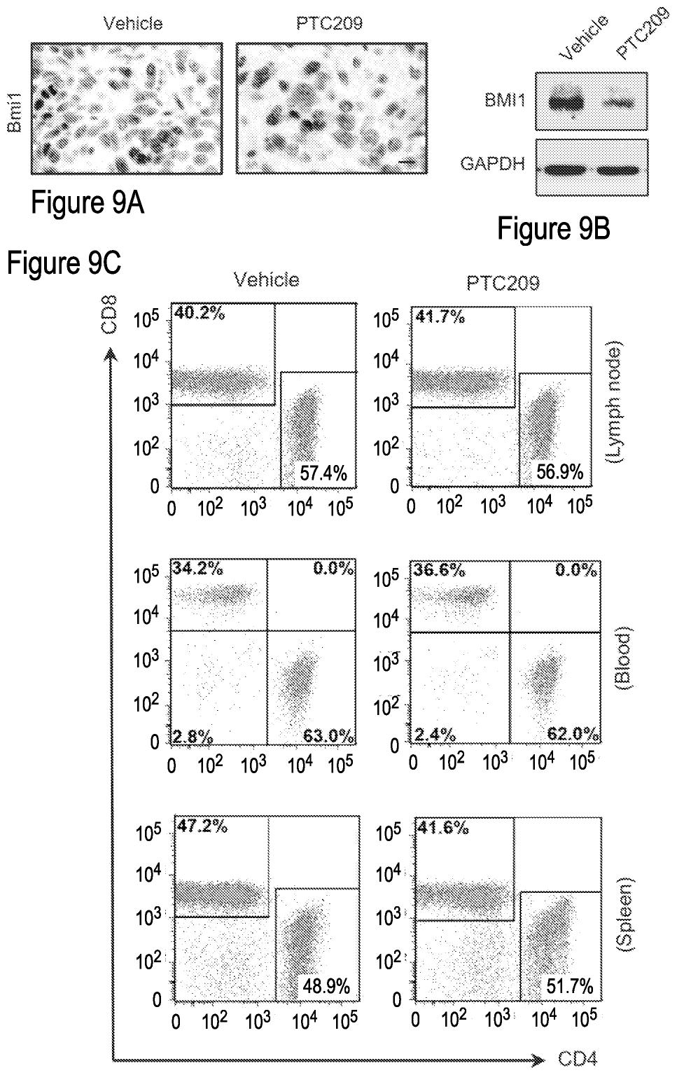

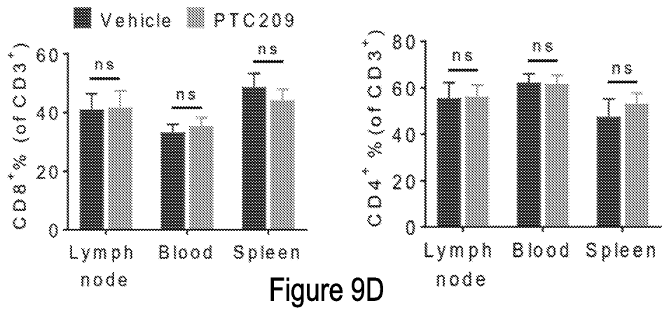

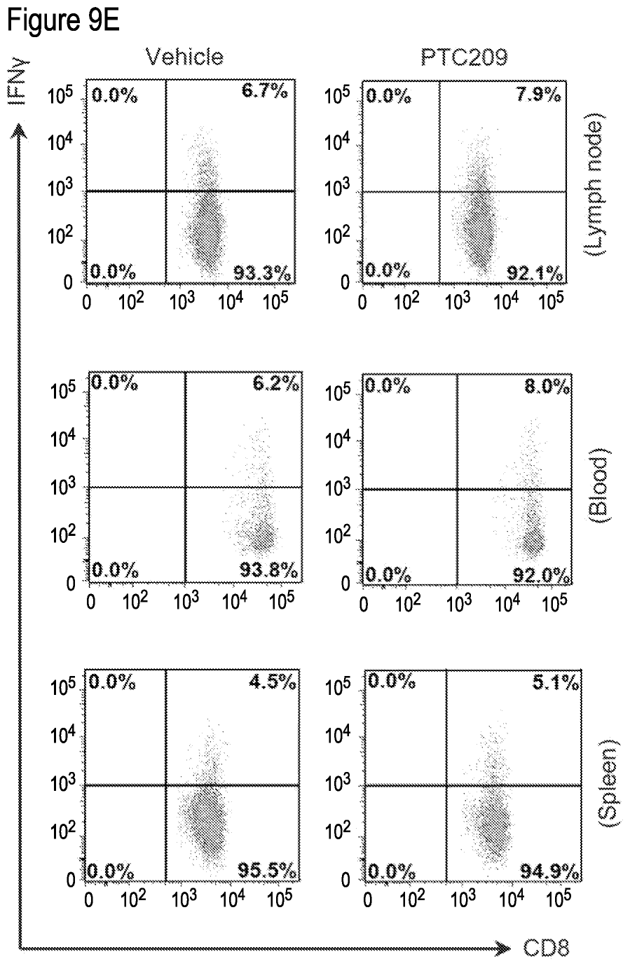

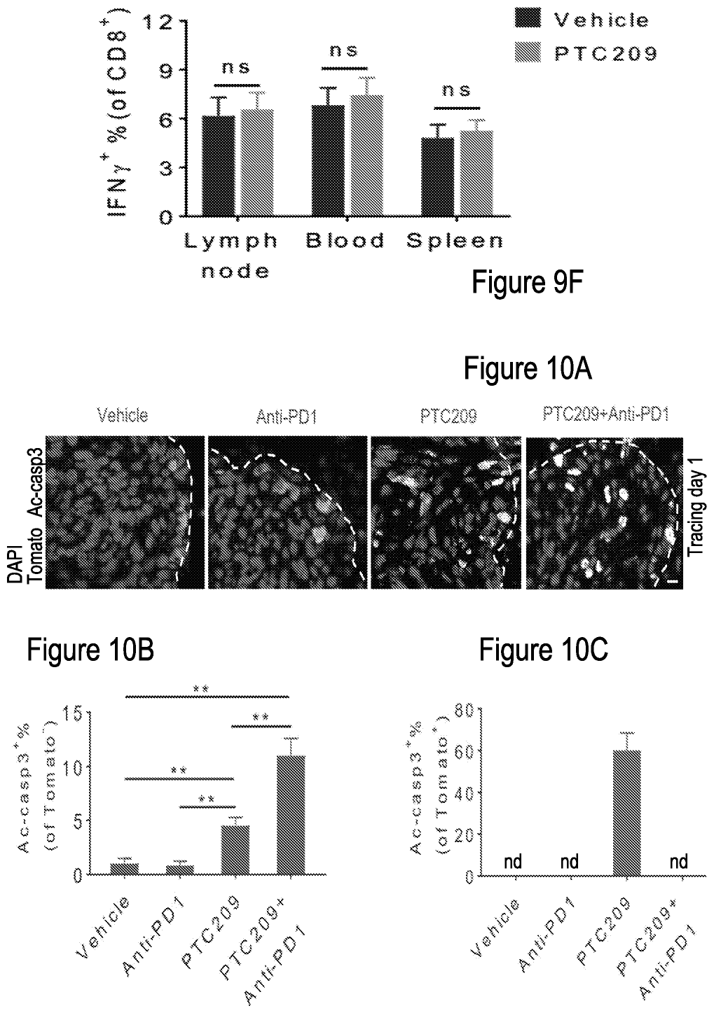

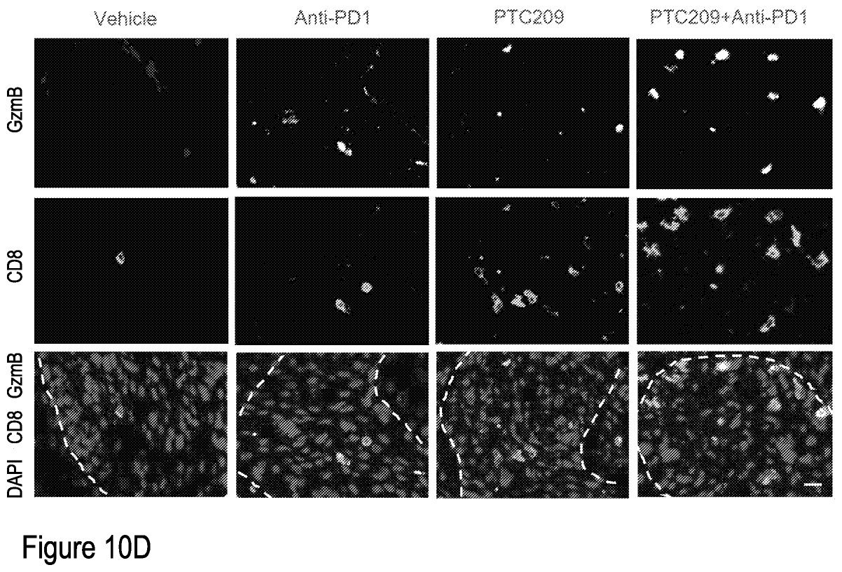

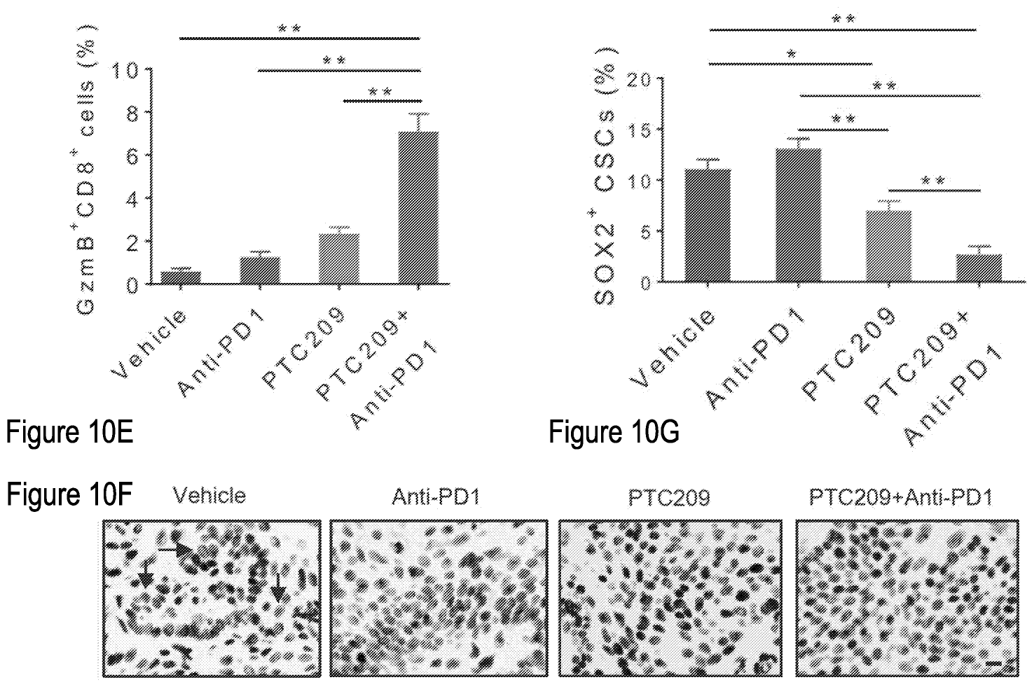

To explore whether targeting BMI1⁺ CSCs augmented PD1 blockade therapy, the specific Bmi1 inhibitor PTC209, which has be shown to effectively destroy BMI1⁺ CSCs, was used. Reduction of BMI1 expression by PTC209 in tumors was confirmed by immunostaining (FIG. 9A) and Western blot (FIG. 9B). Flow cytometry analysis showed that there was no significant alteration in the percentage of CD8+ T cells and IFNγ-produced CD8+ T cells in the lymph nodes, blood and spleen after PTC209 treatment (FIGS. 9C-9F). Tumor-bearing Bmi1^{CreER};Rosa^{tdTomato} mice were treated with anti-PD1, PTC209, anti-PD1 plus PTC209, or control vehicle. PTC209 plus anti-PD1 significantly reduced more lesion surface areas compared with PTC209 alone (FIGS. 2A and 2B). Histological analysis found that anti-PD1 plus PTC209 significantly reduced HNSCC numbers, areas and invasiveness compared with PTC209 or anti-PD1 (FIGS. 2C-2E). Consistently, anti-Ac-casp3 staining revealed that PTC209 plus anti-PD1 potently induced apoptosis in HNSCC compared with PTC209 or anti-PD1 (FIGS. 2F ad 2G). Whether apoptosis was induced in CSCs or non-stem tumor cells was further examined. While anti-PD1 did not induce apoptosis in CSCs, PTC209 was able to induce apoptosis in Tomato⁺ CSCs. However, apoptotic Tomato⁺ cells after PTC209 plus anti-PD1 treatment were not found, probably because the combination treatment potently induced apoptosis in BMI1⁺ CSCs and impaired their self-renewal (FIGS. 10A-10C). PTC209 plus anti-PD1 effectively eliminated the majority of lymph node metastasis of HNSCC as determined by anti-PCK immunostaining (FIGS. 2H-2J). Unexpectedly, immunostaining found that PTC209, but not anti-PD1, could induce CD8+ T cell infiltration in tumors, which were further significantly increased in HNSCC treated with PTC209 plus anti-PD1 (FIGS. 2K and 2L). To assess the functional activity of CD8+ T cells, the expression of the Granzyme-B (GzmB) in CD8+ T cells was analyzed. Immunostaining found that anti-PD1 plus PTC209 also significantly increased GzmB+CD8+ T cell infiltration in tumors (FIGS. 10D and 10E). In vivo labeling showed that PTC209 was able to reduce BMI1+ CSCs while anti-PD1 did not. In sharp contrast to anti-PD1 plus cisplatin, in vivo labeling showed that anti-PD1 plus PTC209 efficiently eliminated BMI1+ CSCs in HNSCC (FIGS. 2M and 2N). SOX2+ cells have been identified to represent CSCs in the skin SCC, and previous study has shown that most of the BMI1+ cells had increasing SOX2 protein expression in HNSCC (Chen et al., 2017). Because of weak positive staining in some non-stem tumor cells, only SOX2+ cells which were strongly stained were counted. Immunostaining revealed that the number of SOX2+ cells were significantly reduced after PTC209 treatment, which were further decreased in tumors treated with PTC209 plus anti-PD1 (FIGS. 10F and 10G).

### Requirement of Intratumoral CD8⁺ T Cells for Anti-Tumor Immunity Induced by BMI1 Inhibitor Plus Anti-PD1

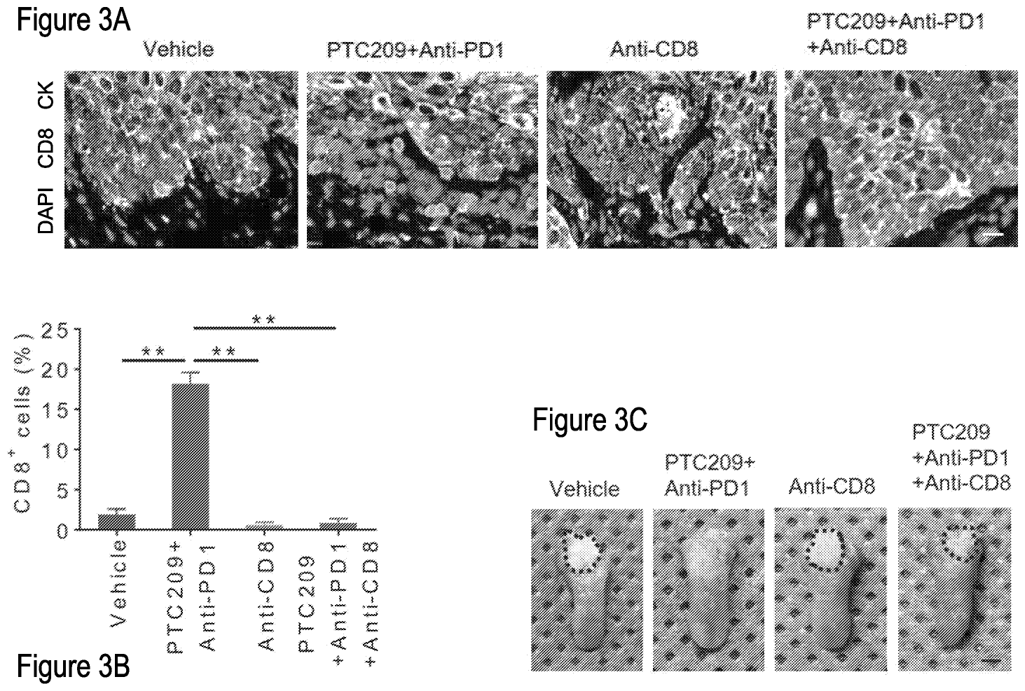

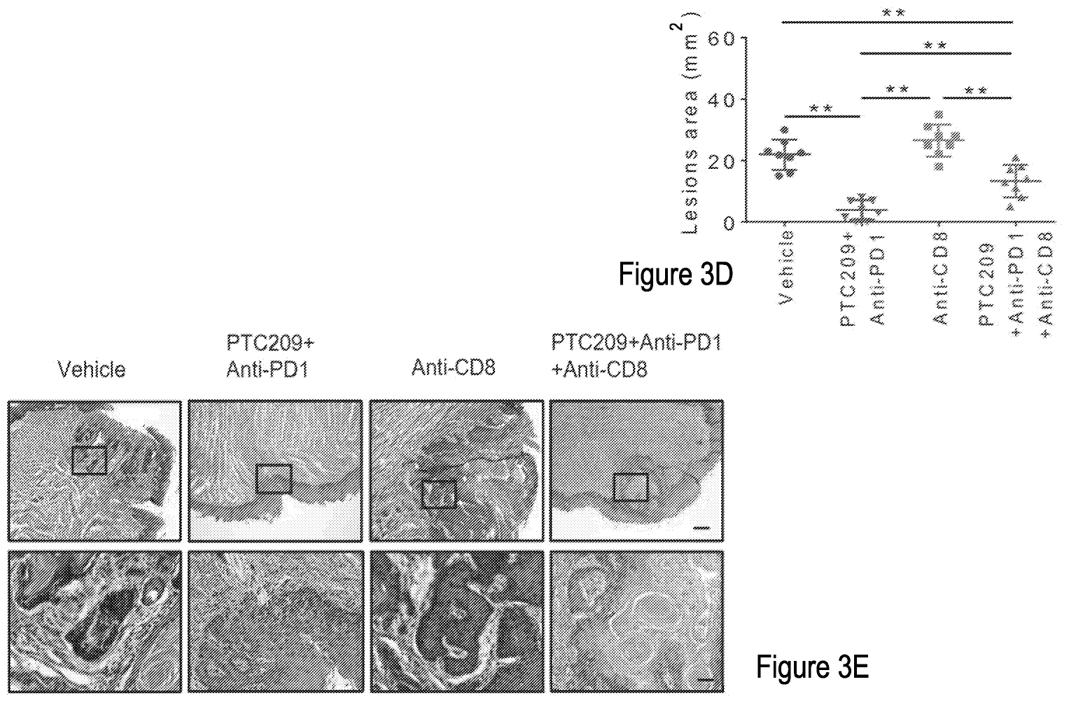

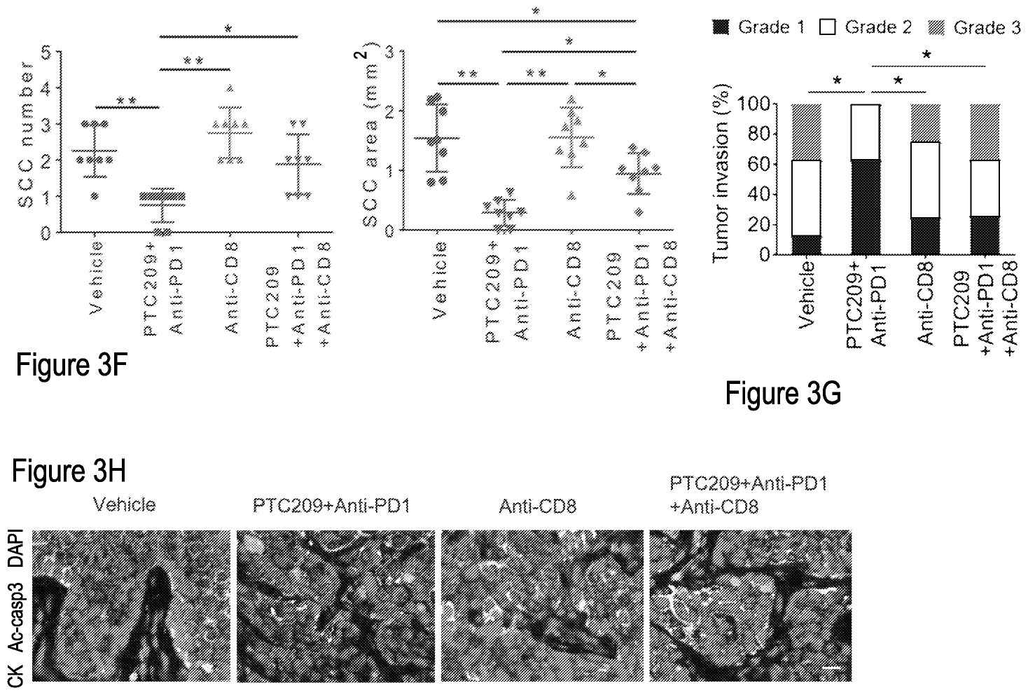

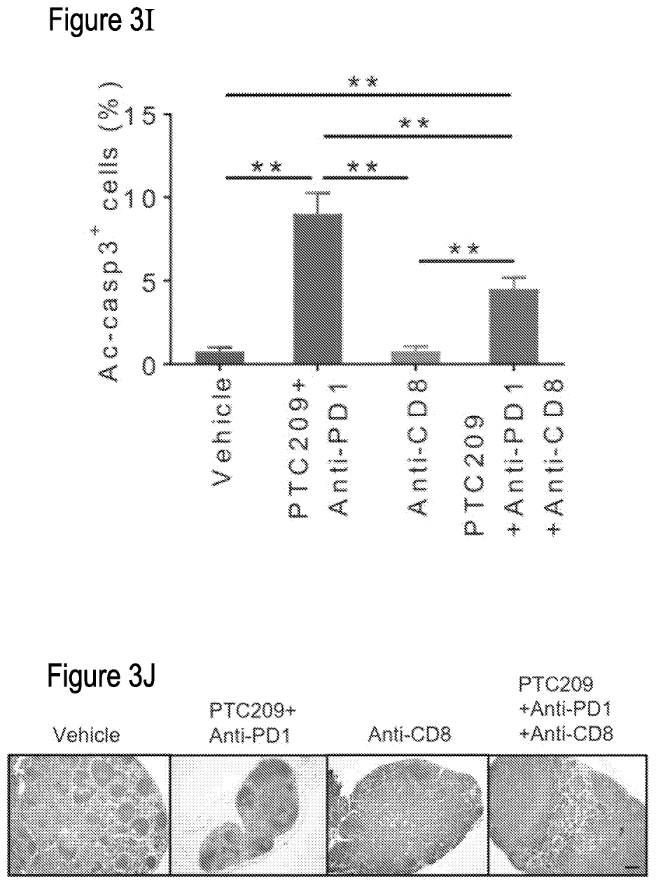

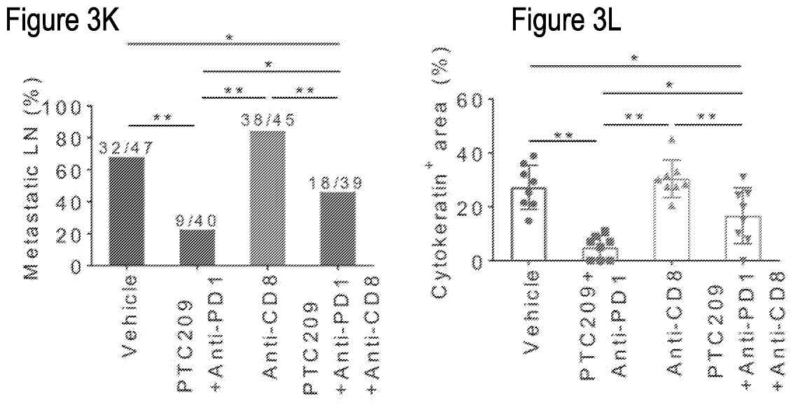

To test whether intratumoral CD8⁺ cells were required for PTC209 plus anti-PD1-mediated anti-tumor immunity, we concurrently treated tumor-bearing mice with anti-CD8 antibodies to immunodeplete CD8⁺ cells. Immunostaining showed that anti-CD8 significantly abrogated CD8⁺ T cells infiltration in HNSCC induced by PTC-209 plus anti-PD1 (FIGS. 3A and 3B). Anti-CD8 significantly restored visible lesion areas inhibited by PTC-209 plus anti-PD1 (FIGS. 3C and 3D). Histological analysis found that anti-CD8 significantly reversed the inhibition of HNSCC growth by PTC209 plus anti-PD1 (FIGS. 3E-3G). Consistently, anti-CD8 attenuated PTC209 plus anti-PD1-induced apoptosis in HNSCC (FIGS. 3H and 3I). Furthermore, anti-CD8 also significantly lessened the inhibition of lymph node metastasis of HNSCC mediated by PTC209 plus anti-PD1 (FIGS. 3J-3L).

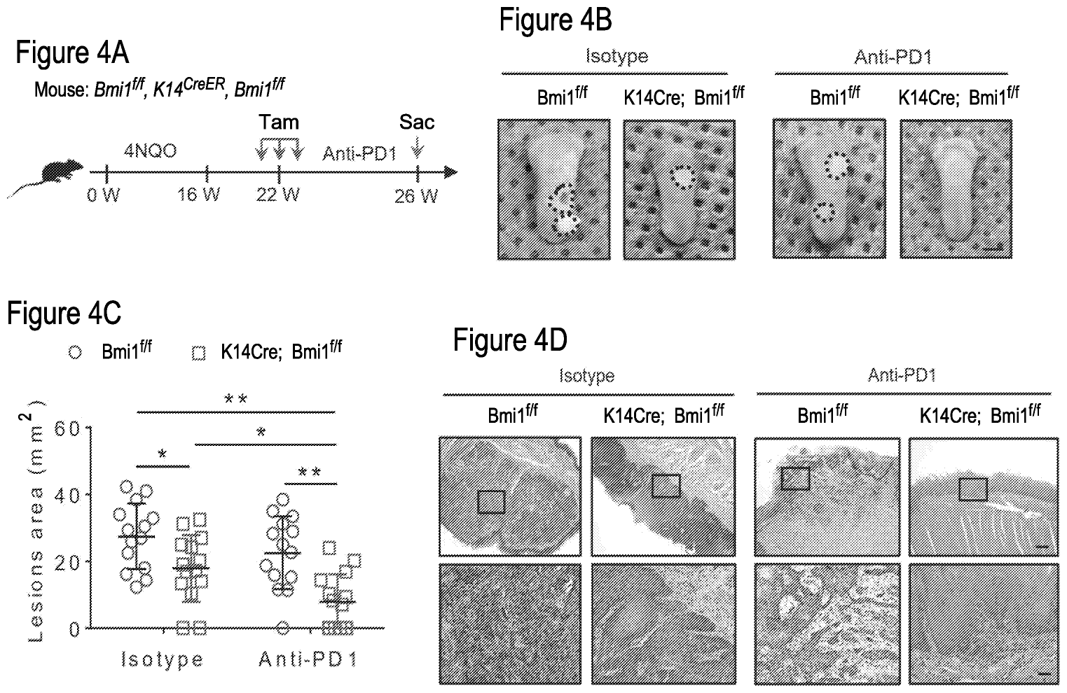

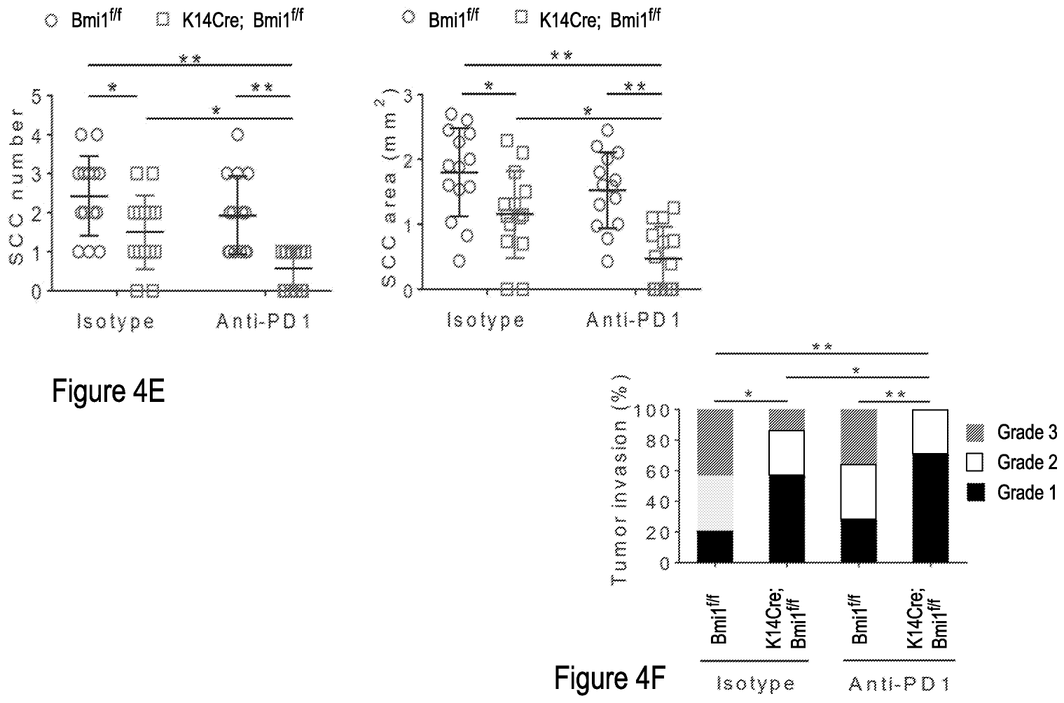

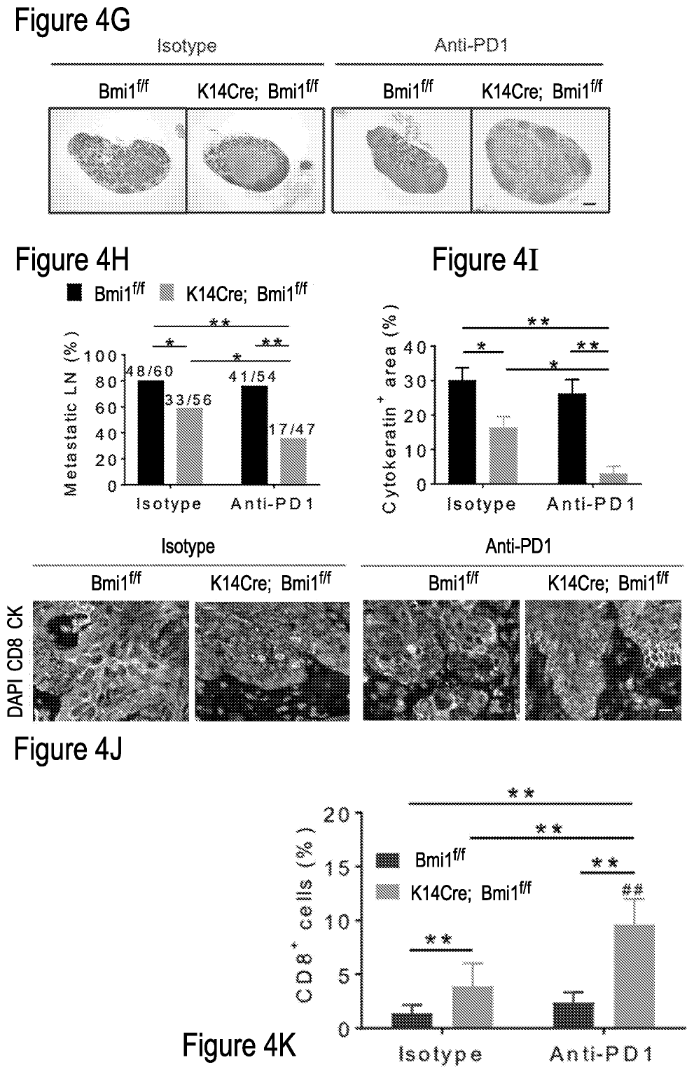

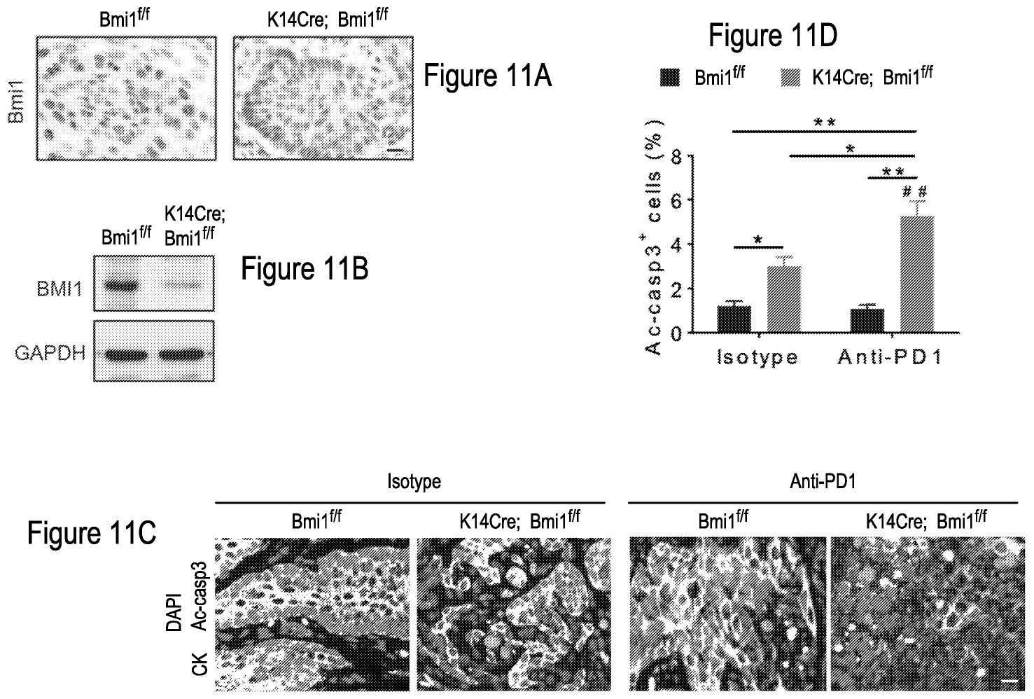

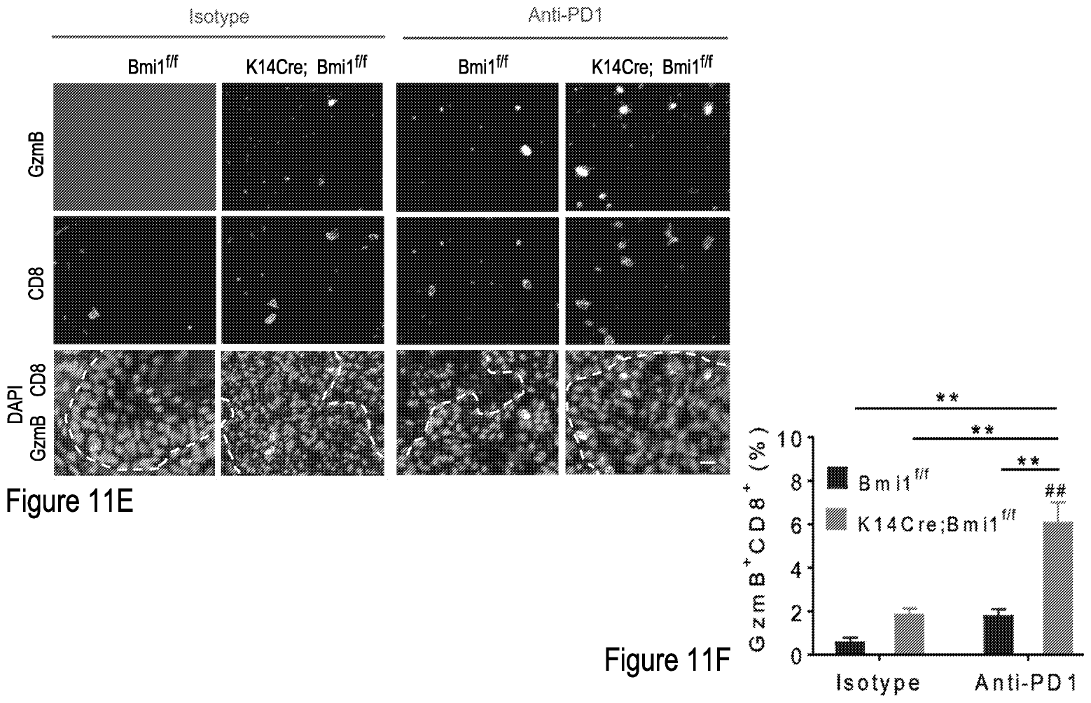

To further determine whether targeting tumor cell-intrinsic BMI1 promoted CD8⁺ T cells infiltration, Bmi1^{flox/flox} (Bmi1^{f/f}) mice were crossed with keratin 14-Cre/ERT2 mice (K14CreER) to generate K14^{CreER};Bmi1^{f/f} mice in which epithelial BMI1 can be inducibly deleted by tamoxifen treatment. To achieve efficient recombination activity, three successive applications of tamoxifen were applied to both K14^{CreER};Bmi1^{f/f} and the control Bmi1^{f/f} mice 22 weeks after the initial 4NQO treatment (FIG. 4A). BMI1 knockout (BMI1 KO) in mouse HNSCC was confirmed by immunostaining (FIG. 11A) and Western blot (FIG. 11B). Consistently, whereas BMI1 KO alone reduced the lesion surface areas, BMI1 KO plus anti-PD1 had superior inhibitory effects (FIGS. 4B and 4C). Histological analysis revealed that BMI1 KO plus anti-PD1 significantly inhibited the numbers, areas and invasive grades of HNSCC compared with BMI1 KO or anti-PD1 alone (FIGS. 4D-4F). Consistently, BMI1 KO plus anti-PD1 also potently induced apoptosis in HNSCC (FIGS. 11C and 11D). Moreover, BMI1 KO plus anti-PD1 also exhibited superior inhibitory effects on lymph node metastasis compared with BMI1 KO alone (FIGS. 4G-4I). Importantly, immunostaining revealed that BMI1 KO alone could induce CD8+ T cell infiltration in HNSCC, which was further increased in HNSCC treated with BMI1 KO plus anti-PD1 (FIGS. 4J and 4K). Moreover, BMI1 KO plus anti-PD1 also significantly increased GzmB⁺CD8+ T cells in HNSCC (FIGS. 11E and 11F). Taken together, these findings confirm that the inhibition of BMI1 in tumor cells collaborates with PD1 blockade to inhibit HNSCC invasive growth and metastasis by recruiting and activating CD8+ T cells.

### Activating Tumor Cell-Intrinsic Immune Response by BMI1 Inhibition

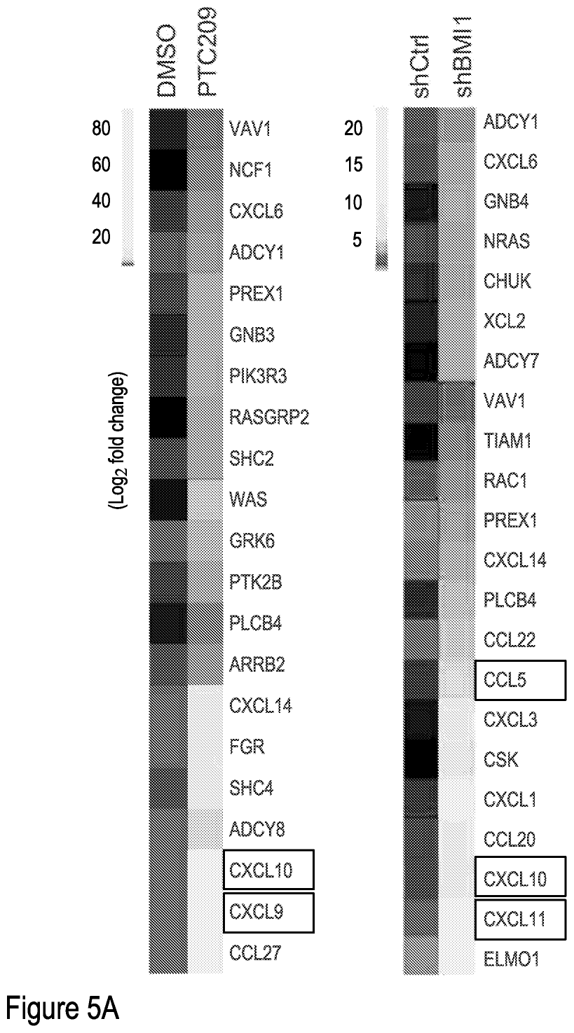

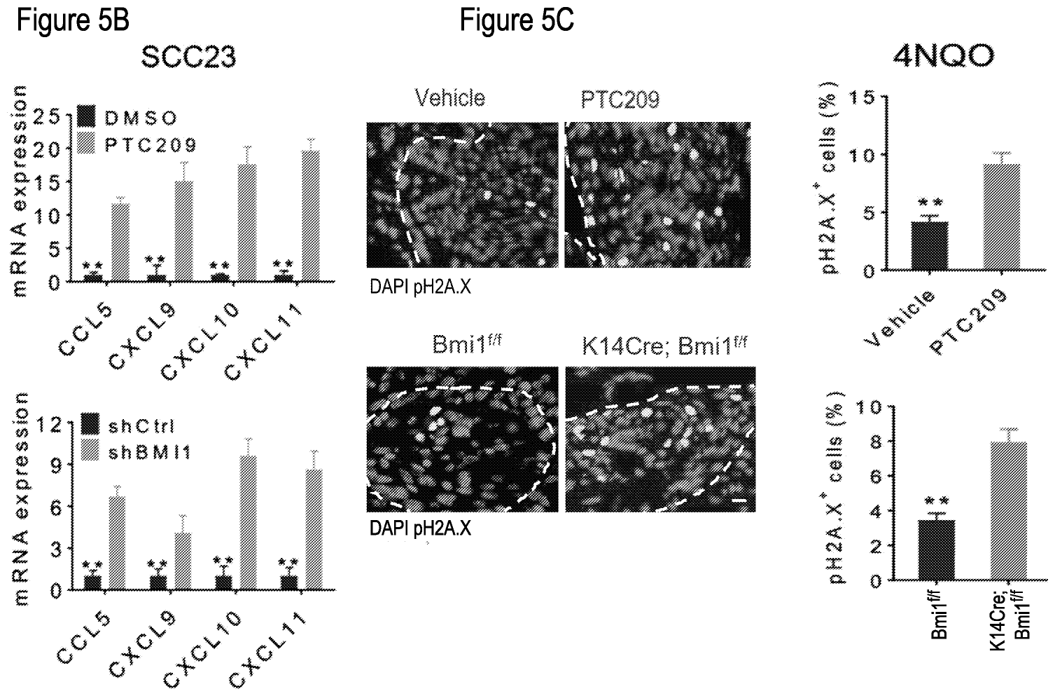

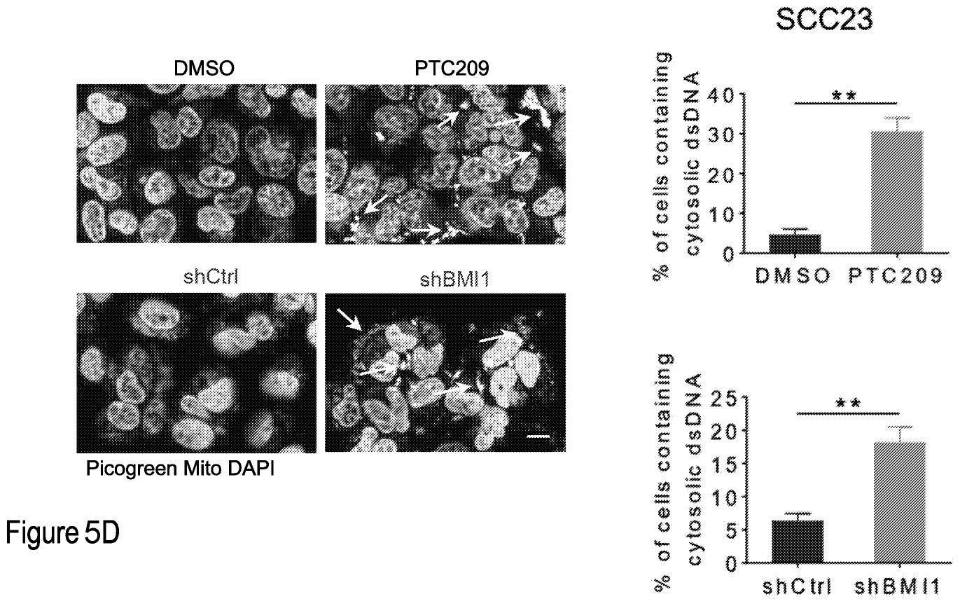

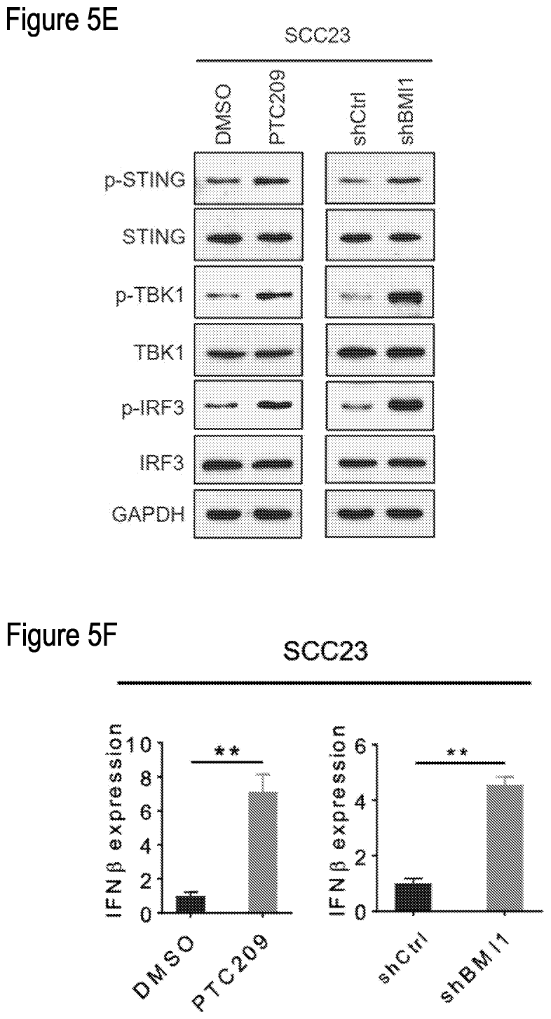

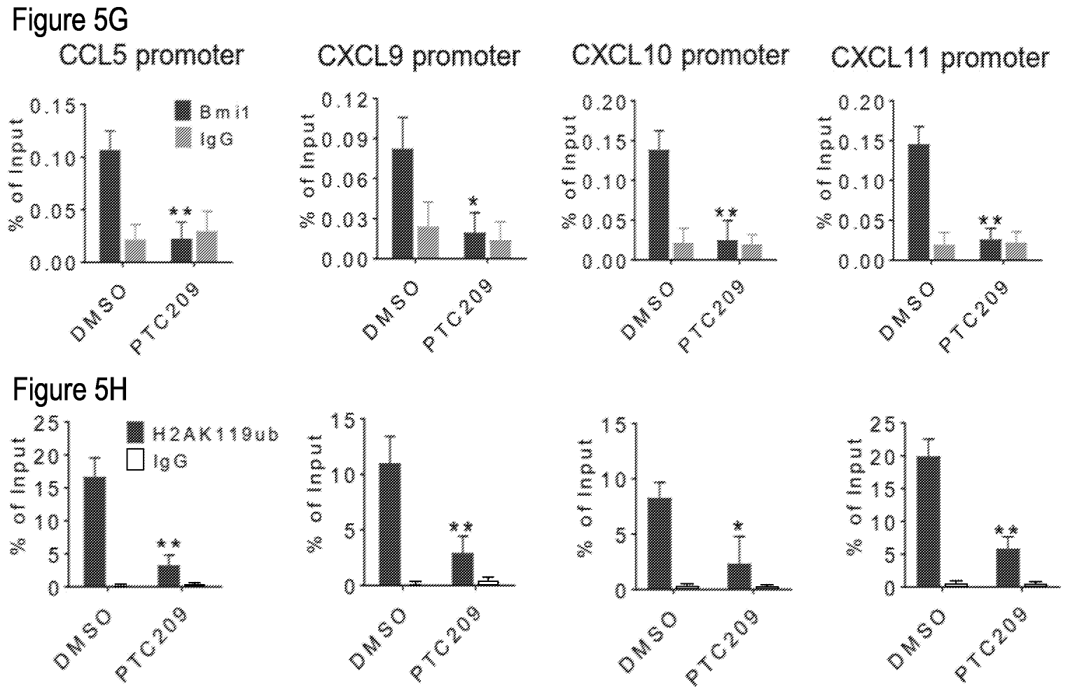

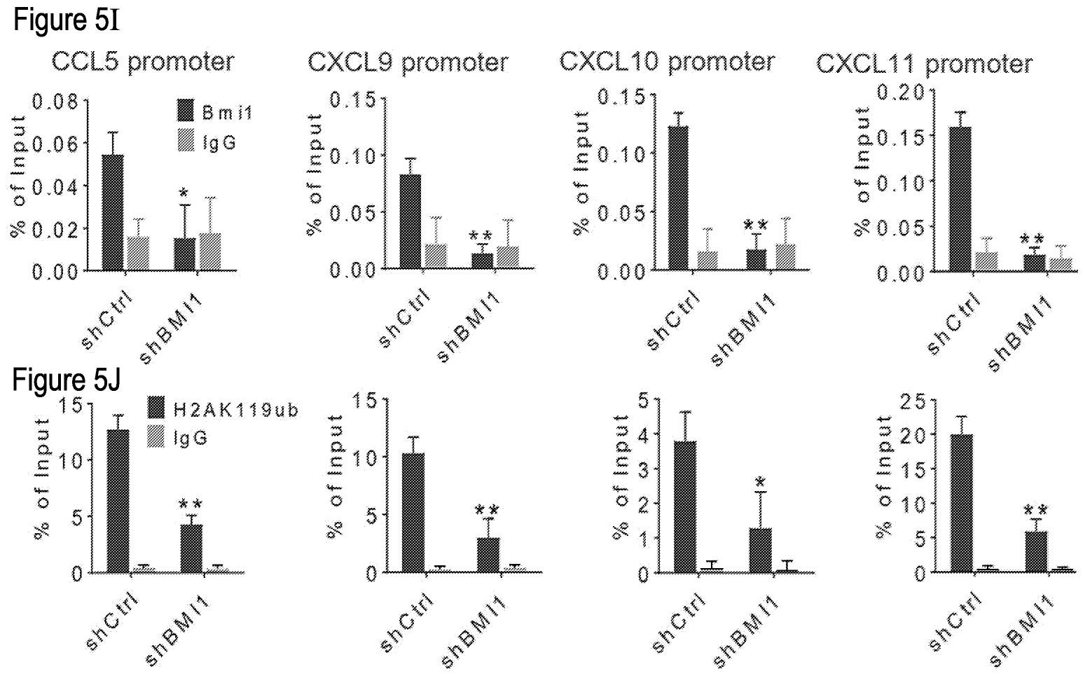

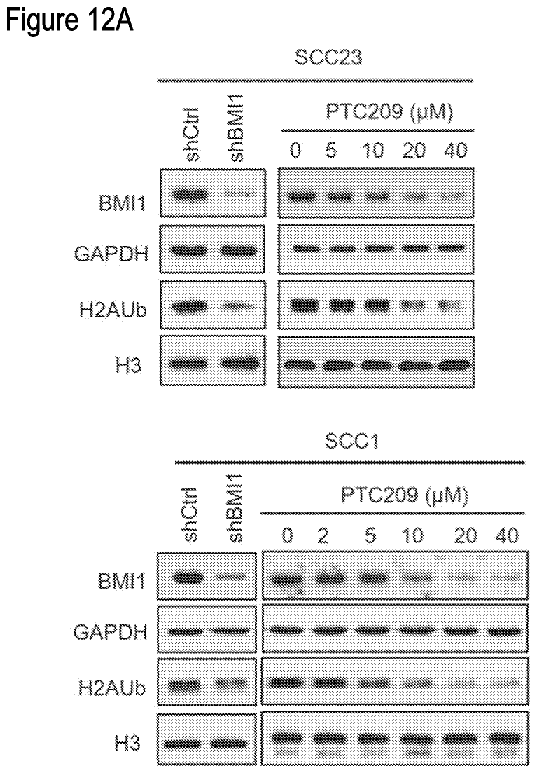

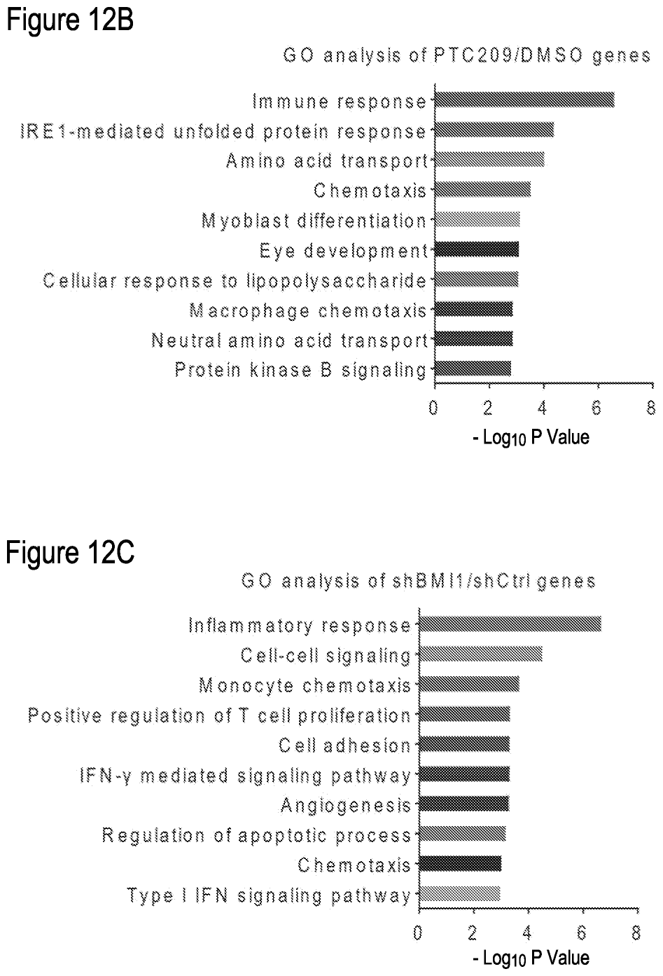

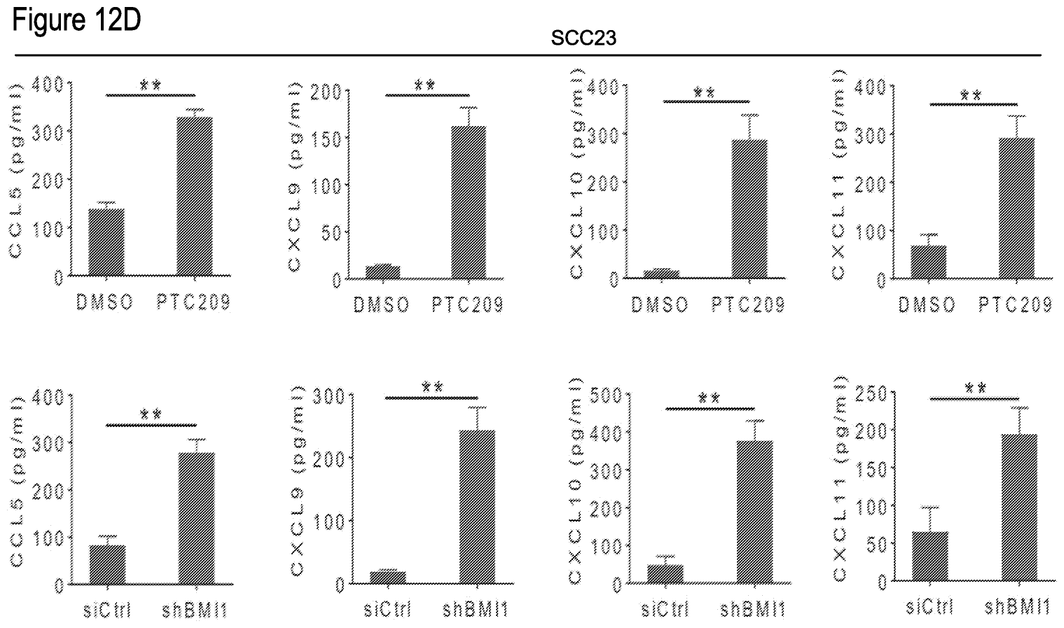

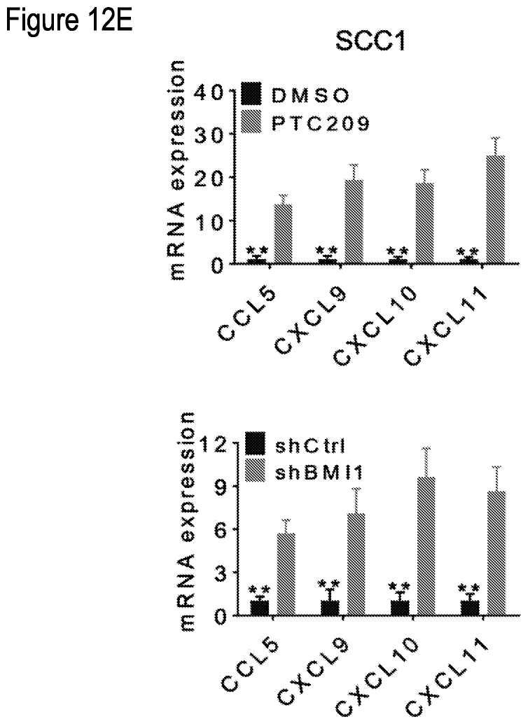

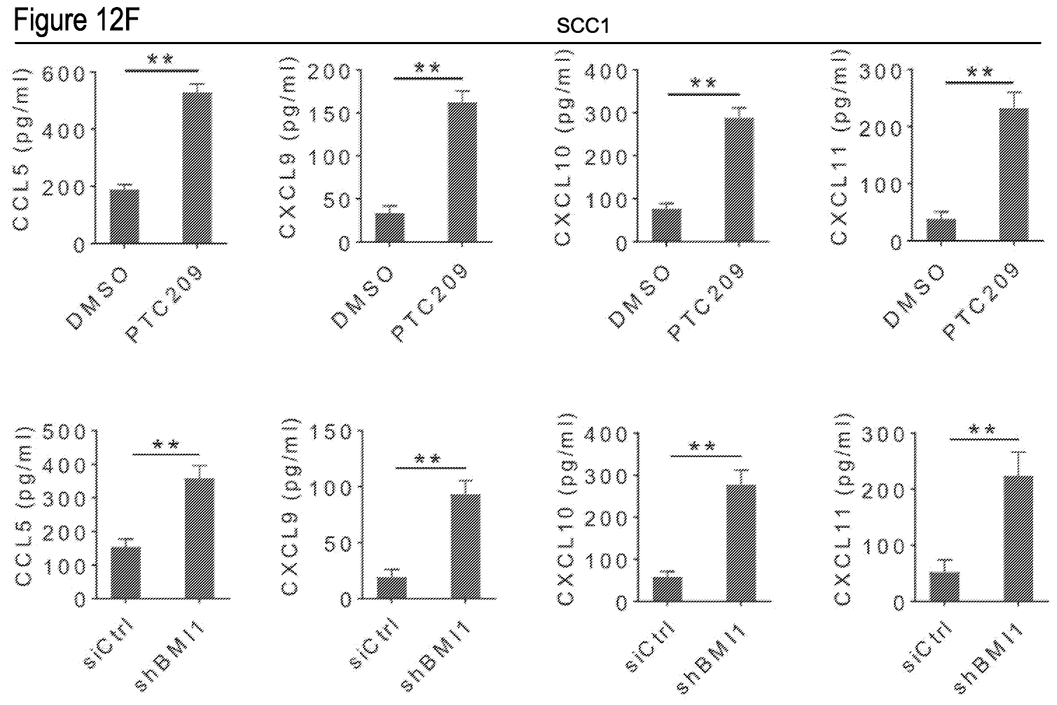

To further elucidate the molecular and epigenetic mechanisms by which BMI1 inhibition recruited CD8+ T cells to augment PD1 blockade therapy, BMI1 expression was knocked down in SCC23 and SCC1 cells using the lentivirus-based short-hairpin RNA for BMI1 (shBMI1). Western blot analysis confirmed that shBMI1 reduced the expression of BMI1. Since BMI1 is required for H2AUb, the level of H2AUb was reduced in SCC23 and SCC1 cells (FIG. 12A). Similarly, PTC209 also reduced BMI1 and H2AUb in SCC23 and SCC1 cells in a dose-dependent manner (FIG. 12A). Next, RNA-Seq was performed to determine whether PTC209 treatment or BMI1 knockdown affected the gene expression in SCC23 cells. Interestingly, GO analysis revealed that the inhibition of BMI1 by PTC209 induced the expression of genes associated with immune response and chemotaxis (FIG. 12B). Similarly, BMI1 knockdown also increased the expression of genes associated with inflammatory response, IFNγ signaling and chemotaxis (FIG. 12C). Heatmap from the RNA-seq data showed that IFN-regulated chemokines (CCL5, CXCL9, CXCL10, and CXCL11), which promote the recruitment of CD8+ T lymphocytes into tumor sites were significantly increased by BMI1 knockdown or PTC209 (FIG. 5A). PTC209 or BMI1 knockdown also significantly increased the expression of CCL5, CXCL9, CXCL10, and CXCL11 in both SCC1 and SCC23 cells (FIGS. 5B and 12D-12F) as determined by both qRT-PCR and enzyme-linked immunosorbent assays (ELISA). Immunostaining was also performed to examine expression of BMI1, CD8, CCL5, and CXCL10 in human HNSCC samples. Immunostaining demonstrated that there was a negative correlation between BMI1 and CD8, CCL5, and CXCL10 in human HNSCC tissues (FIGS. 13A-13C).

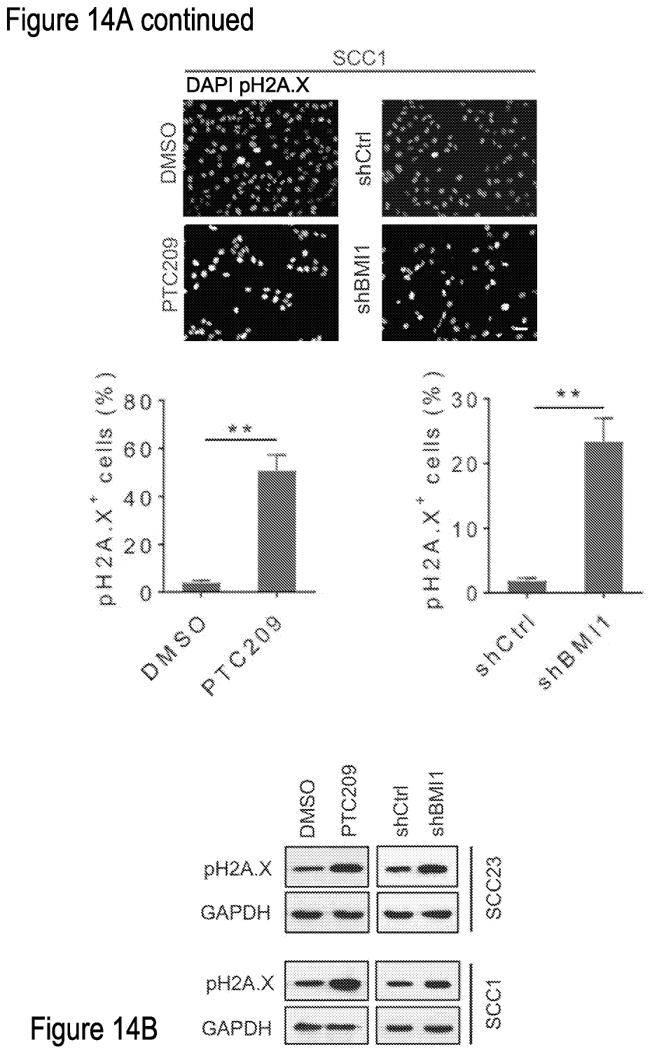

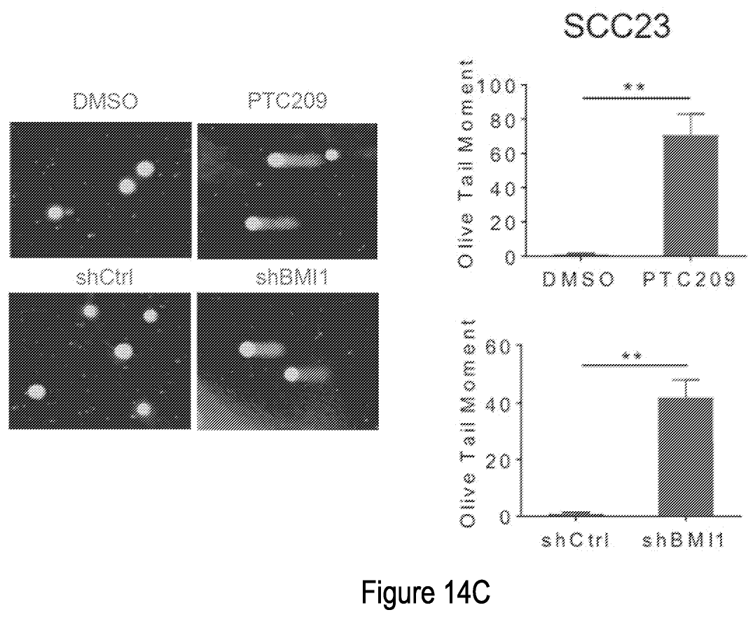

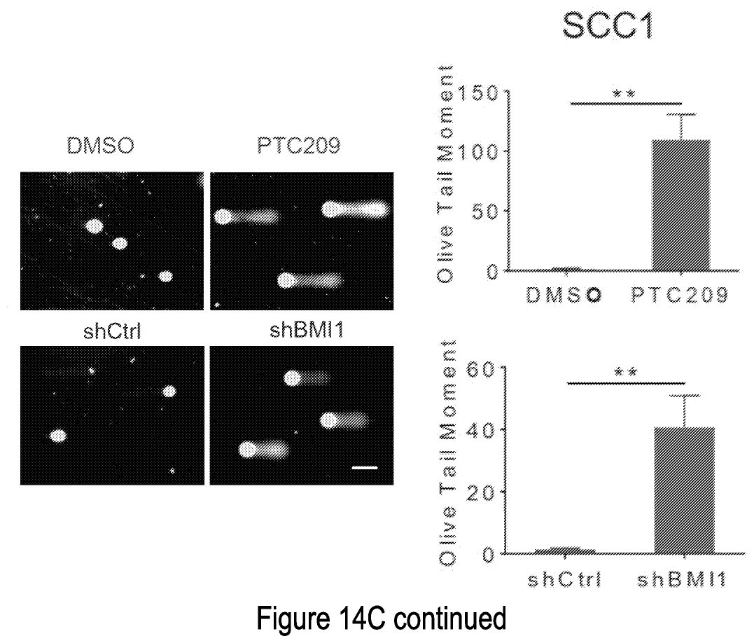

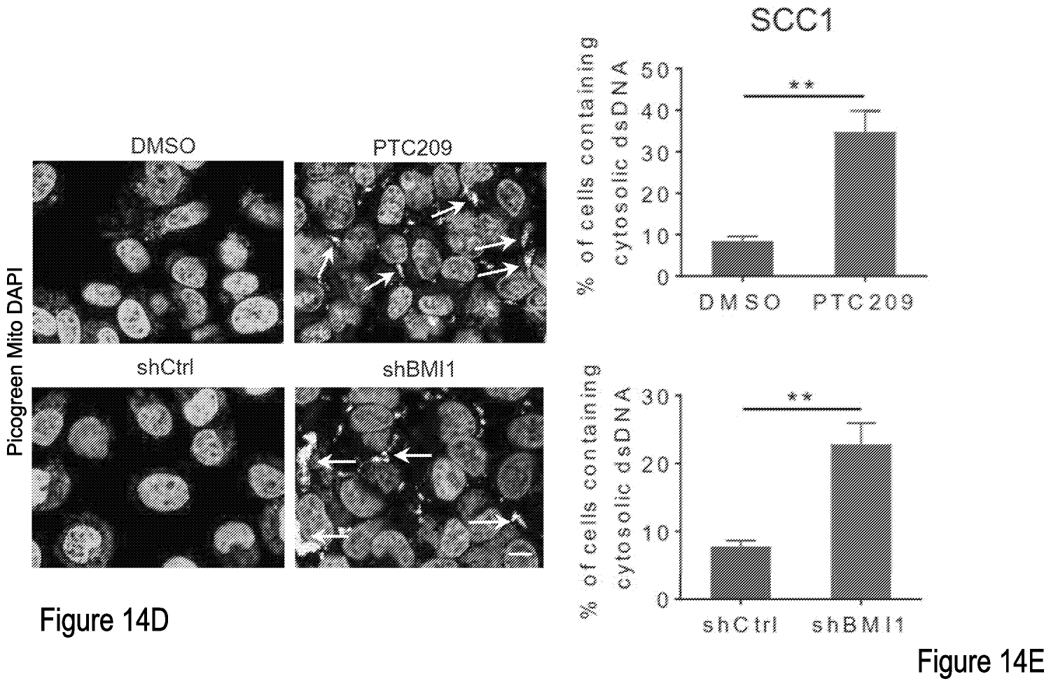

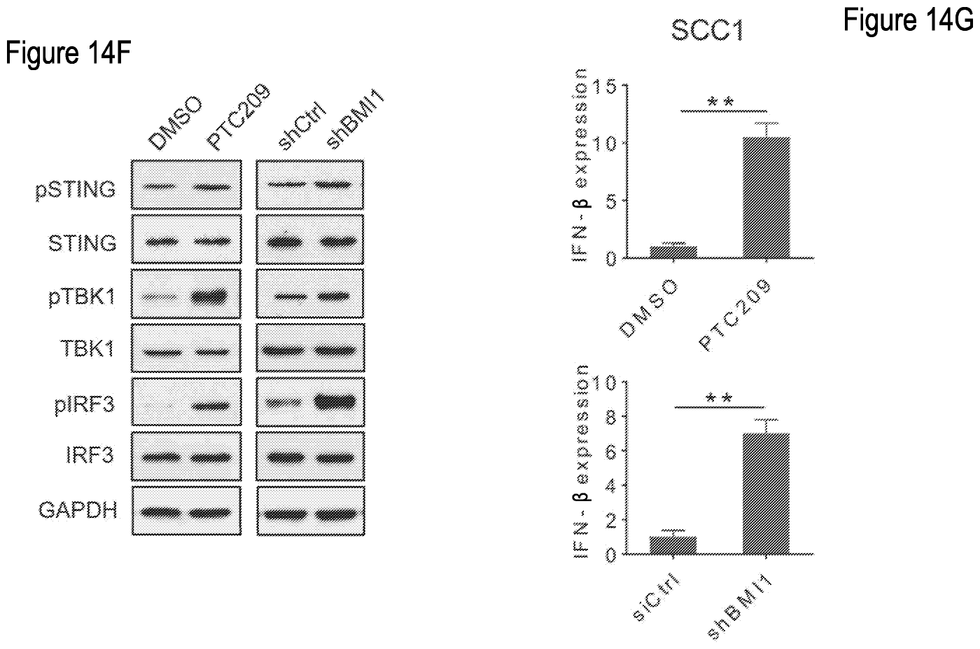

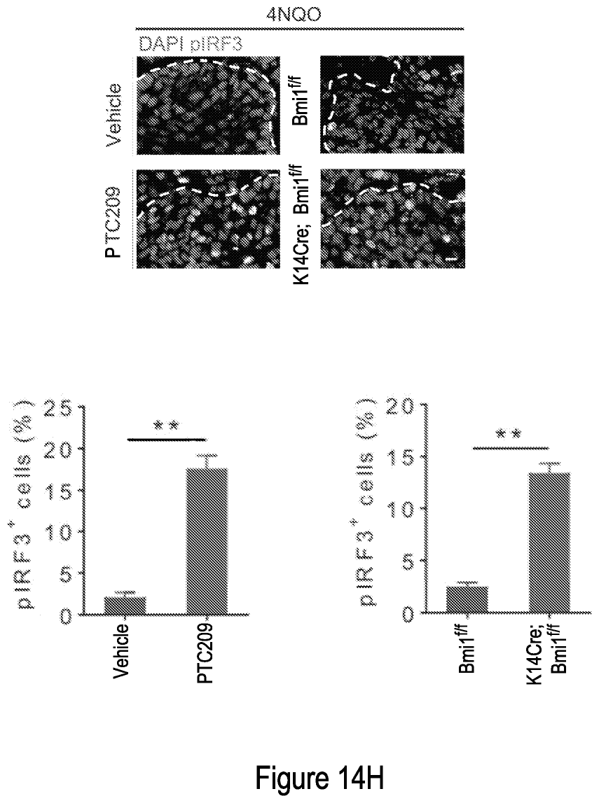

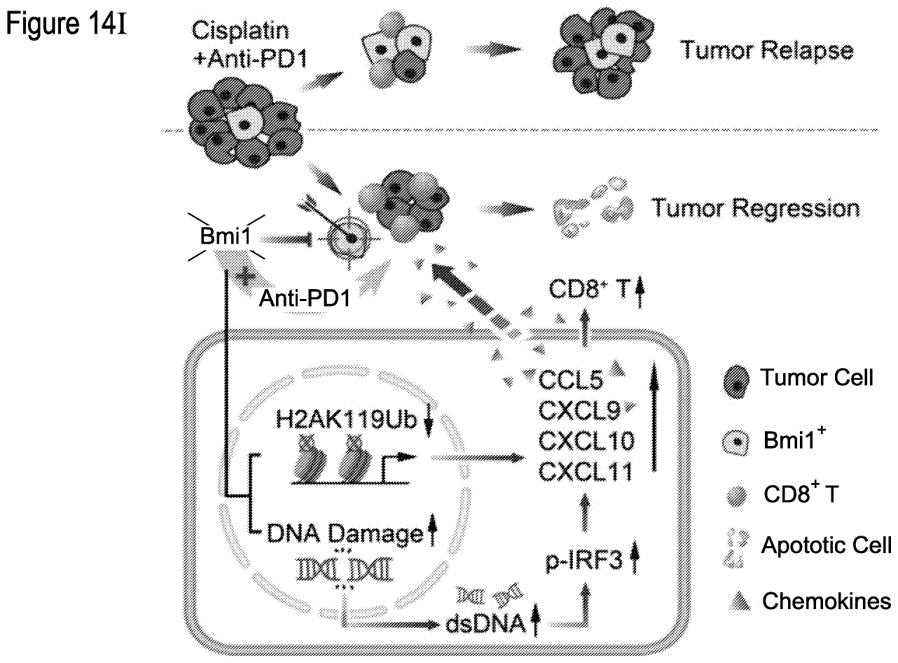

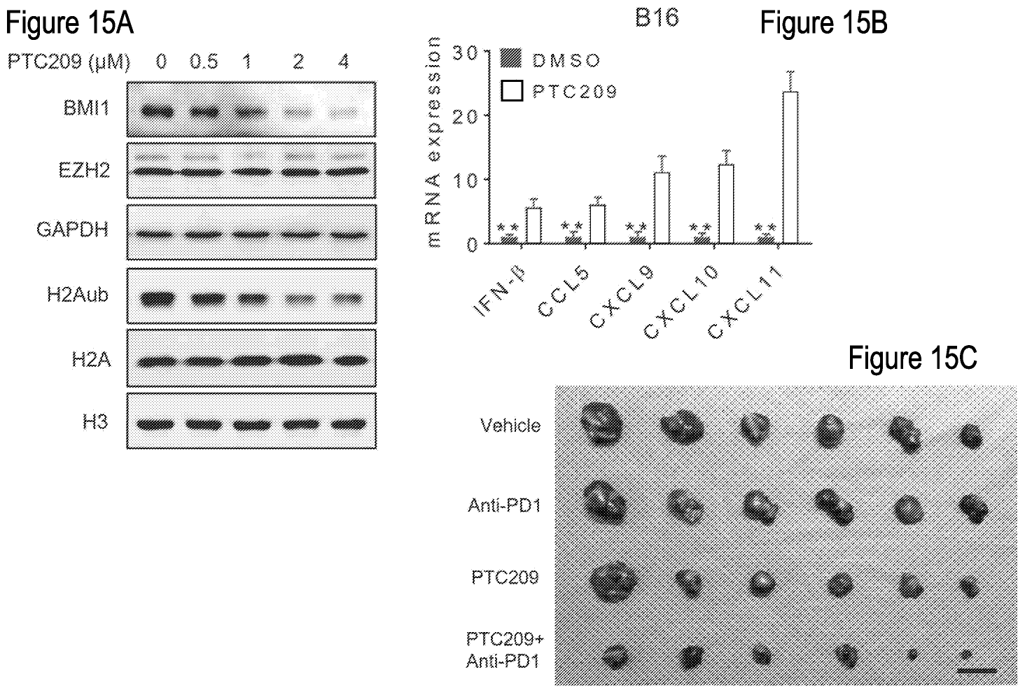

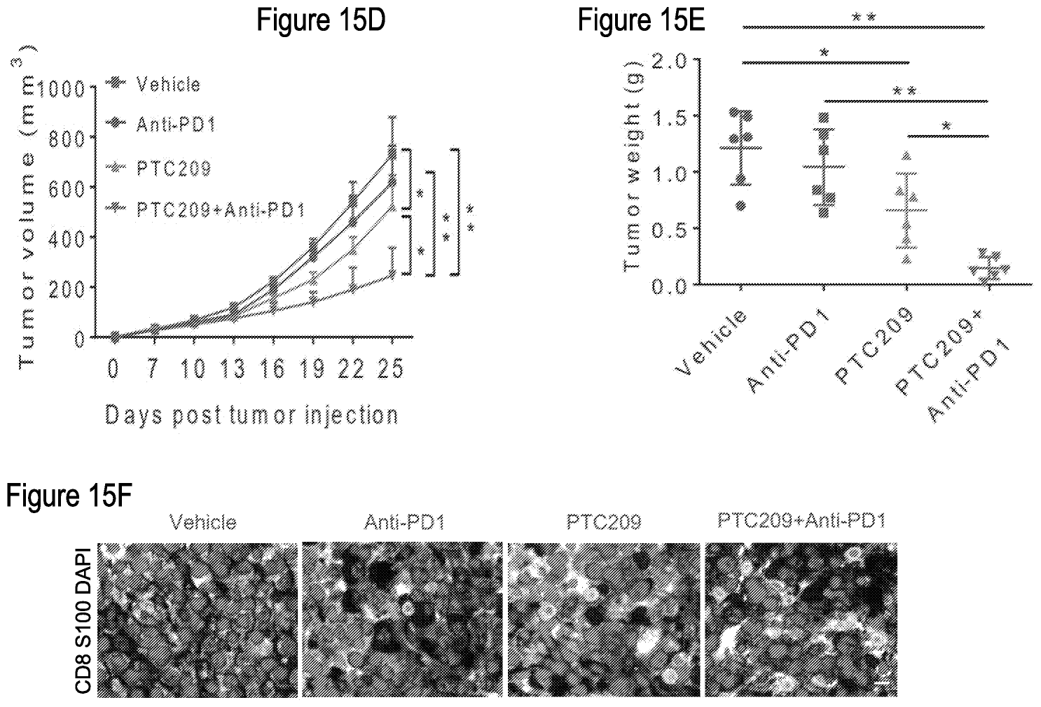

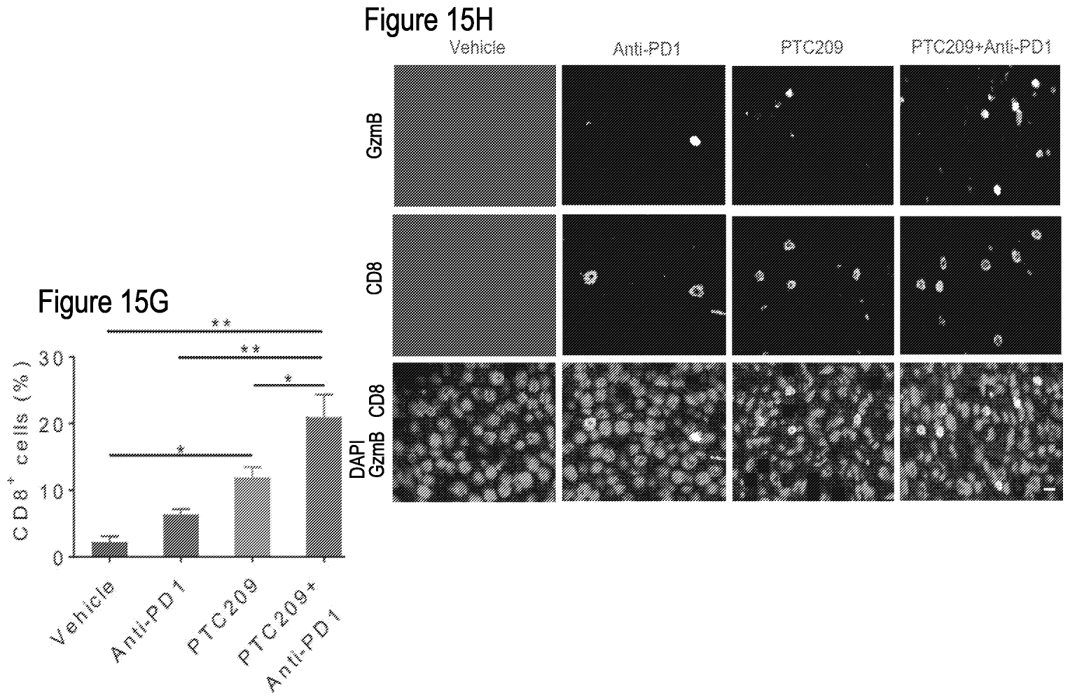

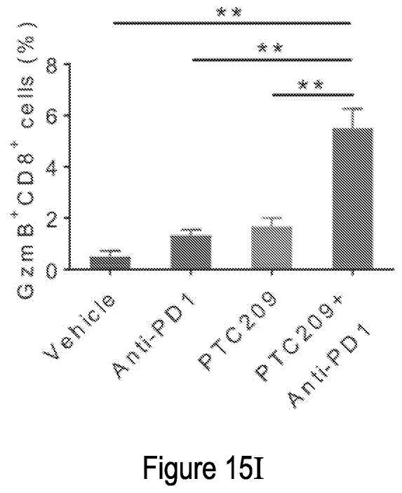

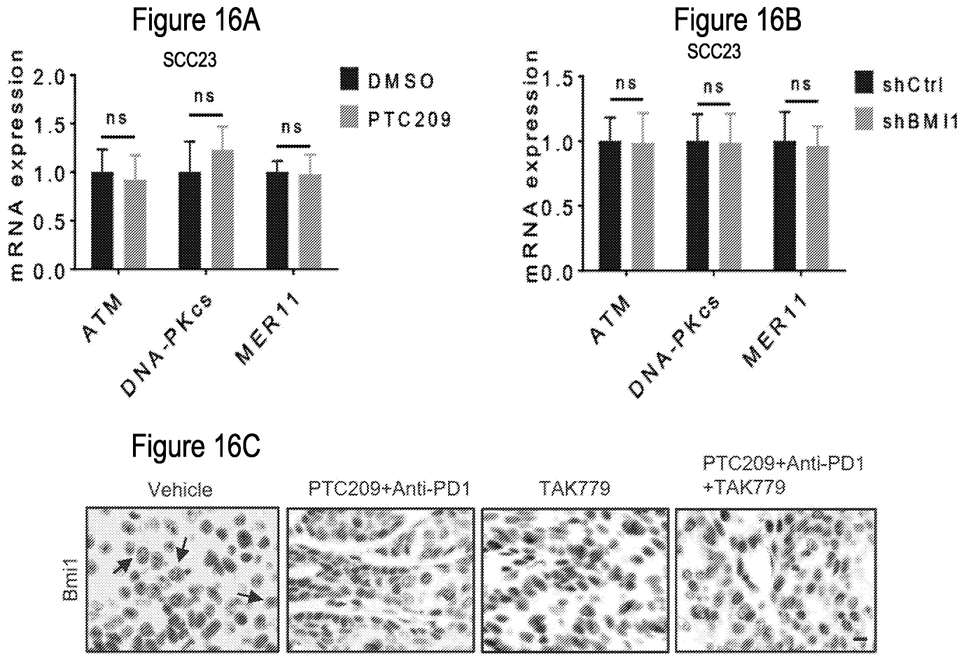

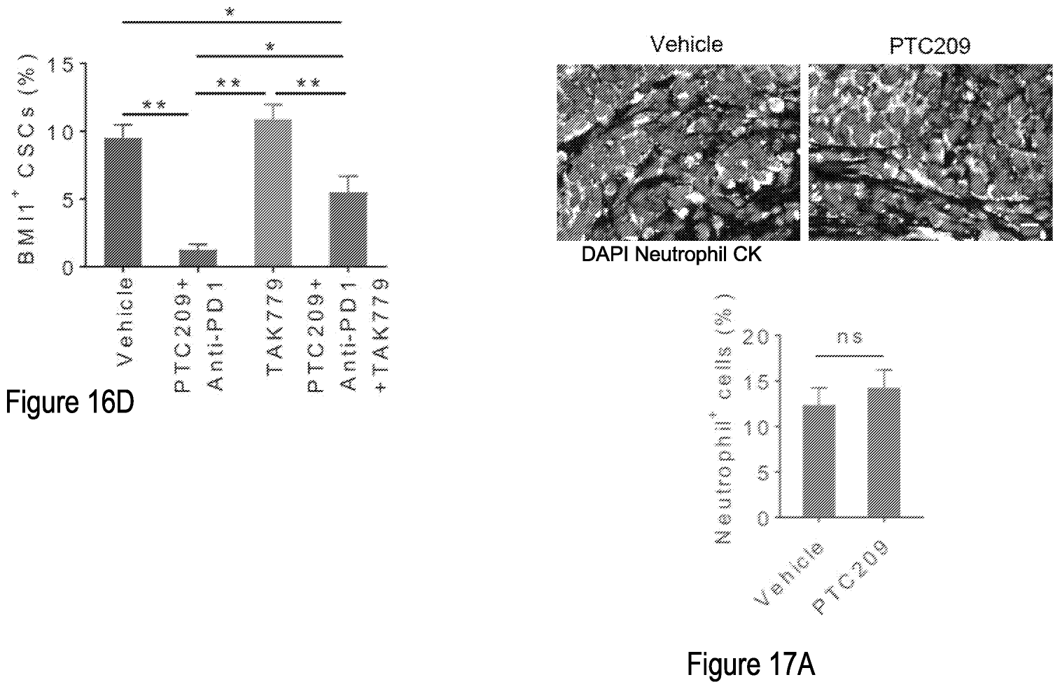

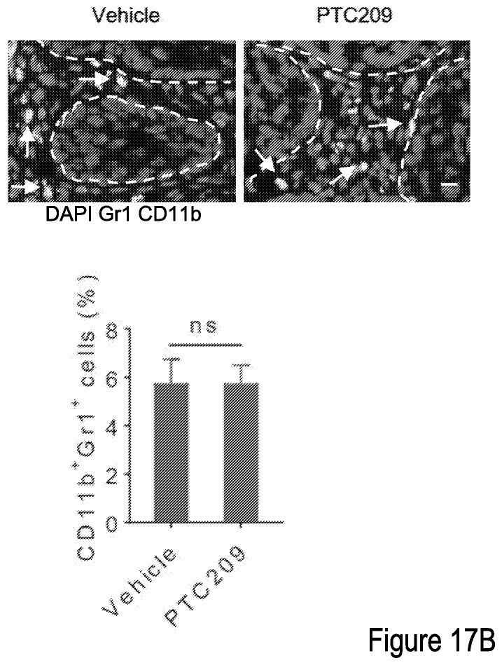

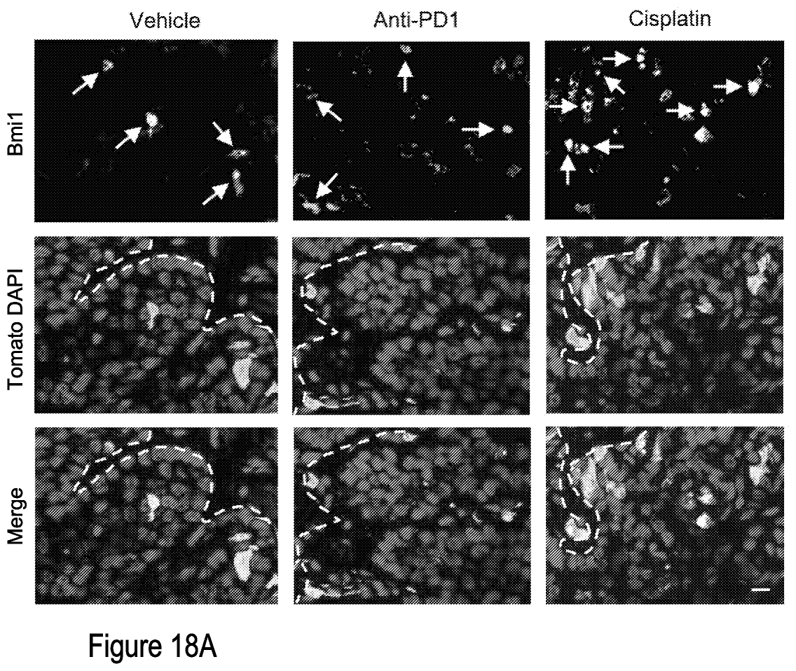

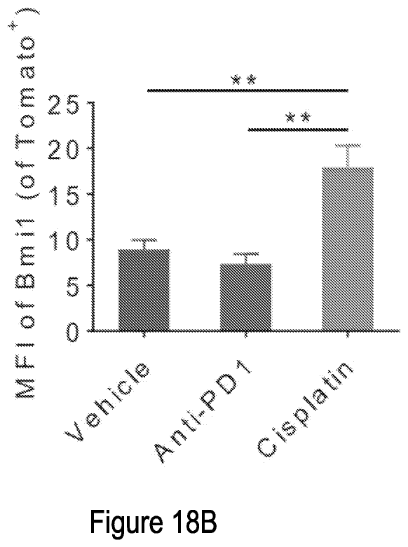

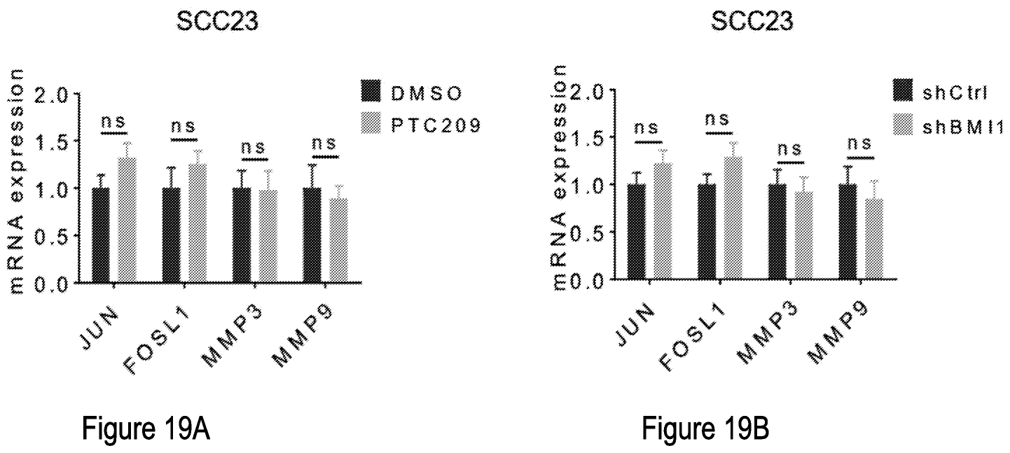

BMI1 has been found to play a regulatory role in DNA damage response and repair. Upon DNA damage, BMI1 is recruited to sites of double-stranded DNA breaks (DSBs) where they promote the ubiquitylation of pH2A.X, thereby facilitating the repair of DSBs by stimulating homologous recombination and non-homologous end joining. To explore how BMI1 inhibition induced IFN-regulated chemokines, pH2A.X (a specific marker for DNA damage) was found to be significantly increased in PTC209-treated HNSCC (FIG. 5C). Similarly, BMI1 KO also increased pH2A.X in HNSCC, indicating that BMI1 inhibition induces DNA damage in HNSCC (FIG. 5C). Furthermore, PTC209 or shBMI1 treatment also increased pH2A.X in SCC23 and SCC1 cells as determined by immunostaining (FIG. 14A) and Western blot (FIG. 14B). PTC209 or shBMI1 treatment also significantly increased the Olive tail moment in SCC23 and SCC1 cells as determined by the alkaline comet assay for the detection of dsDNA damage (FIG. 14C).

To examine whether DNA damages induced by BMI1 inhibition caused the accumulation of cytosolic double strand DNA (dsDNA) in SCC cells, SCC cells were stained with PicoGreen, a dsDNA-specific vital dye. Since PicoGreen also stains mitochondrial DNA, mitochondrial dsDNA was also stained with MitoTracker simultaneously. Multiple PicoGreen staining areas in the cytoplasm of SCC23 and SCC1 cells, which were not overlapped with MitoTracker, were detected upon PTC209 or shBMI1 treatment, indicating that PTC209 and shBMI1 induced the accumulation of cytosolic dsDNA (FIGS. 5D, 14D and 14E). It is well known that cytosolic DNA could activate the cyclic GMP-AMP synthase/stimulator of interferon genes (cGAS-STING) signaling axis by the sequential phosphorylation of STING, TBK1, and IRF3 and subsequently induce transcription of IFN and IFN-regulated chemokines. Western blot analysis showed that BMI1 inhibition induced the phosphorylation of STING, TBK1, and IRF3 in SCC23 and SCC1 cells (FIGS. 5E and 14F). Consistently, BMI1 inhibition also induced IFNβ in SCC23 and SCC1 cells (FIGS. 5F and 14G). pIRF3 was also found to be significantly increased in HNSCC upon PTC209 treatment or BMI1 KO, confirming that BMI1 inhibition activated the cGAS-STING-IRF3 pathway in vivo (FIG. 14H).

Recently, PRC1 was found to activate CCL2 transcription to promote cancer stemness and bone metastasis in prostate cancers by recruiting macrophages and regulatory T cells. However, RNA-seq analysis did not detect BMI inhibition regulated CCL2 transcription in HNSCC. In contrast, BMI1 inhibition induced the transcription of chemokines associated with CD8+ T cell recruitments. Whether BMI1 could repress the transcription of chemokines by H2AUb and whether BMI1 inhibition might erase the repressive H2AUb were further explored by chromatin immunoprecipitation-qPCR (ChIP-qPCR). ChIP-qPCR revealed that BMI1 specifically occupied the promoters of CCL5, CXCL9, CXCL10 and CXCL11. PTC209 treatment significantly reduced the levels of BMI1 on these promoters in SCC23 cells (FIG. 5G). Consistently, the levels of H2AUb marks on the promoters of CCL5, CXCL9, CXCL10 and CXCL11 were significantly decreased in SCC23 cells by PTC209 (FIG. 5H). Furthermore, shBMI1 also confirmed that BMI1 was present on the promoters of CCL5, CXCL9, CXCL10 and CXCL11 and that knockdown of BMI1 reduced the levels of H2AUb on their promoters (FIGS. 51 and 5J), indicating that BMI1 inhibition could also de-repress chemokine expression intrinsically. Taken together, these results suggest that BMI1 inhibition stimulated chemokines in tumor cells by two interrelated mechanisms: inducing cGAS-STING signaling to activate IRF3-mediated transcription and erasing repressive H2AUb marks on the promoter of chemokines.

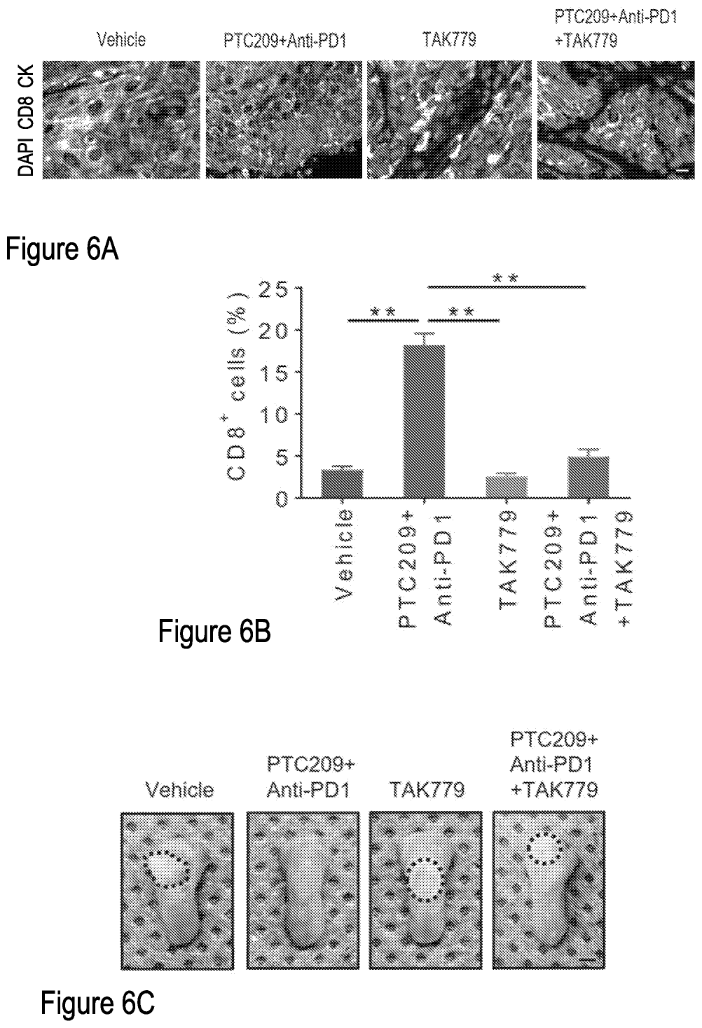

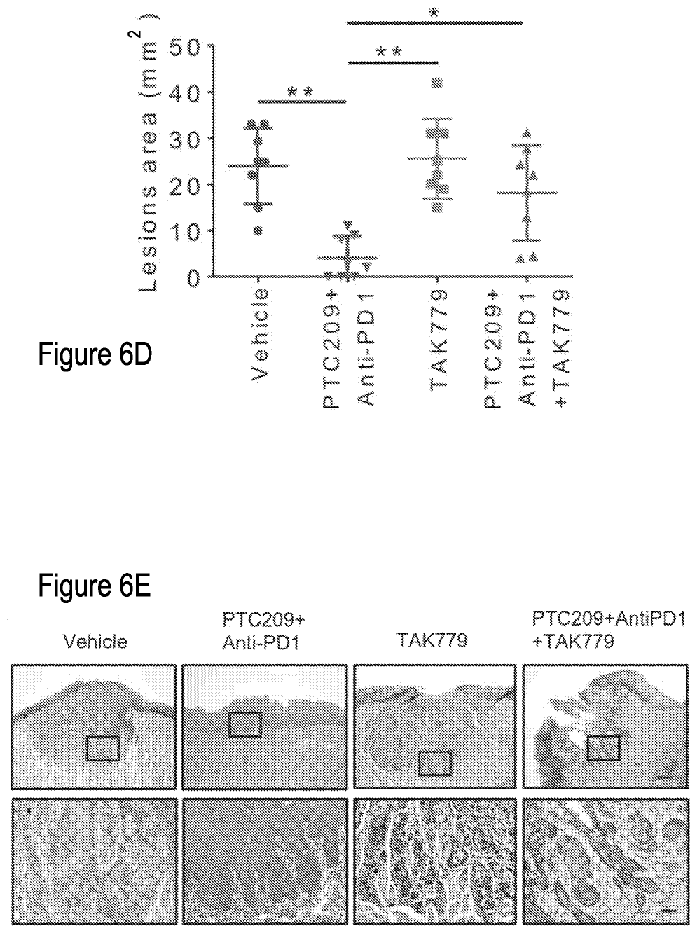

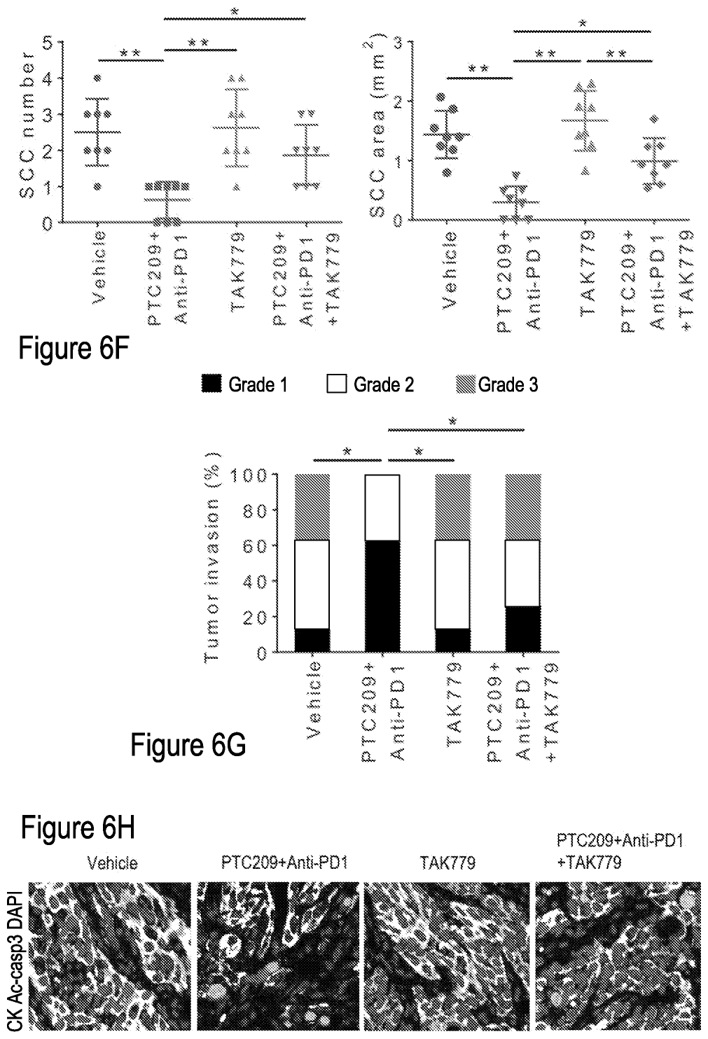

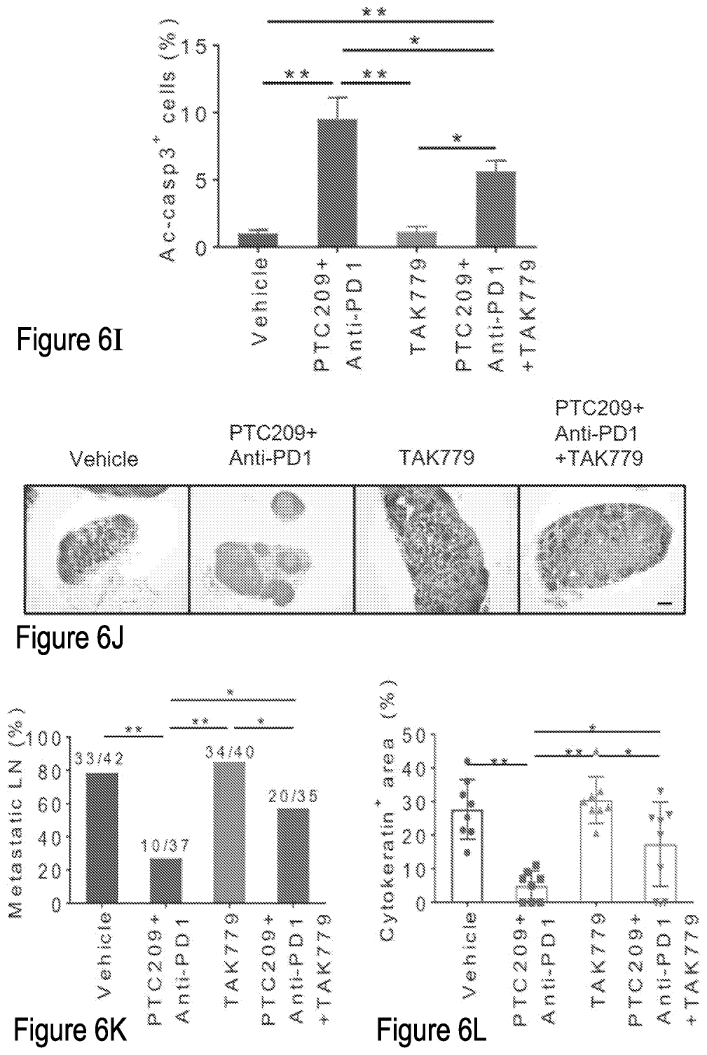

To test whether IFN-regulated chemokines and the recruitment of CD8+ T cells were required for anti-PD1 plus PTC209-mediated anti-tumor immunity, tumor-bearing mice were concurrently treated with TAK779, an inhibitor of CCR5 and CXCR3, which are the receptors for CCL5, CXCL9, CXCL10 and CXCL11, respectively. Immunostaining showed that TAK779 significantly abrogated CD8+ T cells infiltration in HNSCC induced by anti-PD1 plus PTC209 (FIGS. 6A and 6B). TAK779 significantly restored visible lesion areas inhibited by anti-PD1 plus PTC209 (FIGS. 6C and 6D). Histological analysis found that TAK779 significantly reversed the inhibition of HNSCC growth by anti-PD1 plus PTC209 (FIGS. 6E-6G). Consistently, TAK779 attenuated apoptosis in HNSCC induced by PTC209 plus anti-PD1 (FIGS. 6H and 6I). TAK779 also significantly lessened the inhibition of lymph node metastasis of HNSCC mediated by anti-PD1 plus PTC209 (FIGS. 6J-6L).

### BMI1 Inhibitor Plus Anti-PD1 Prevents Relapse of HNSCC

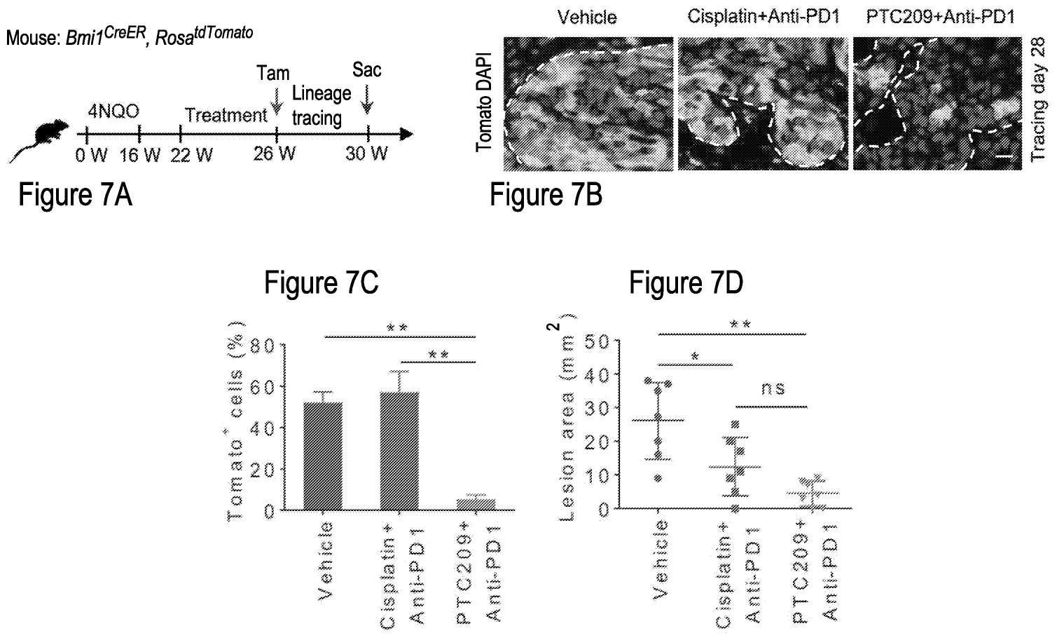

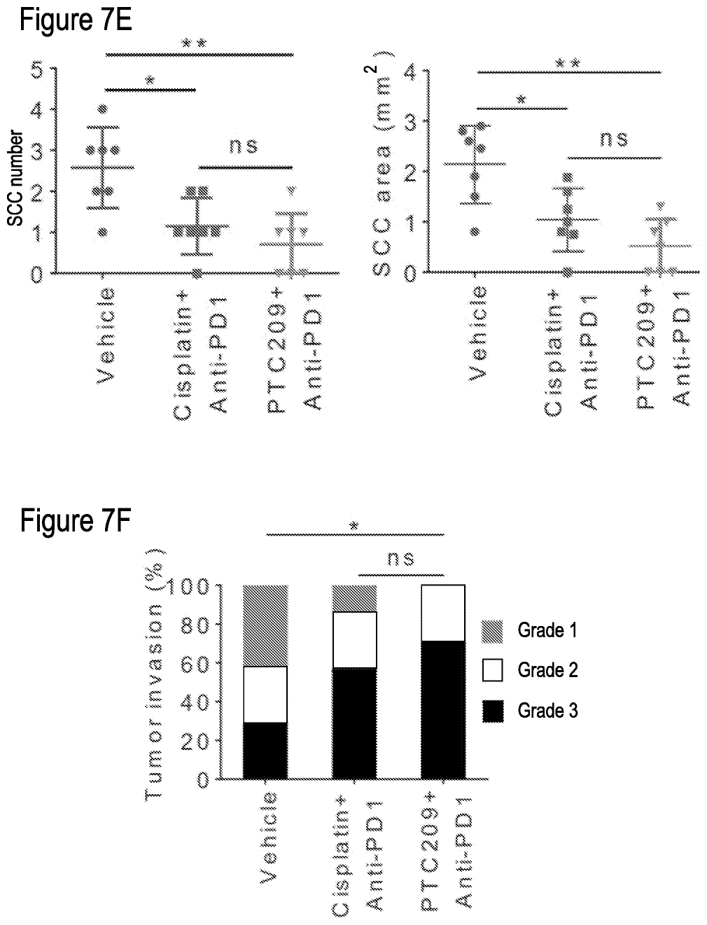

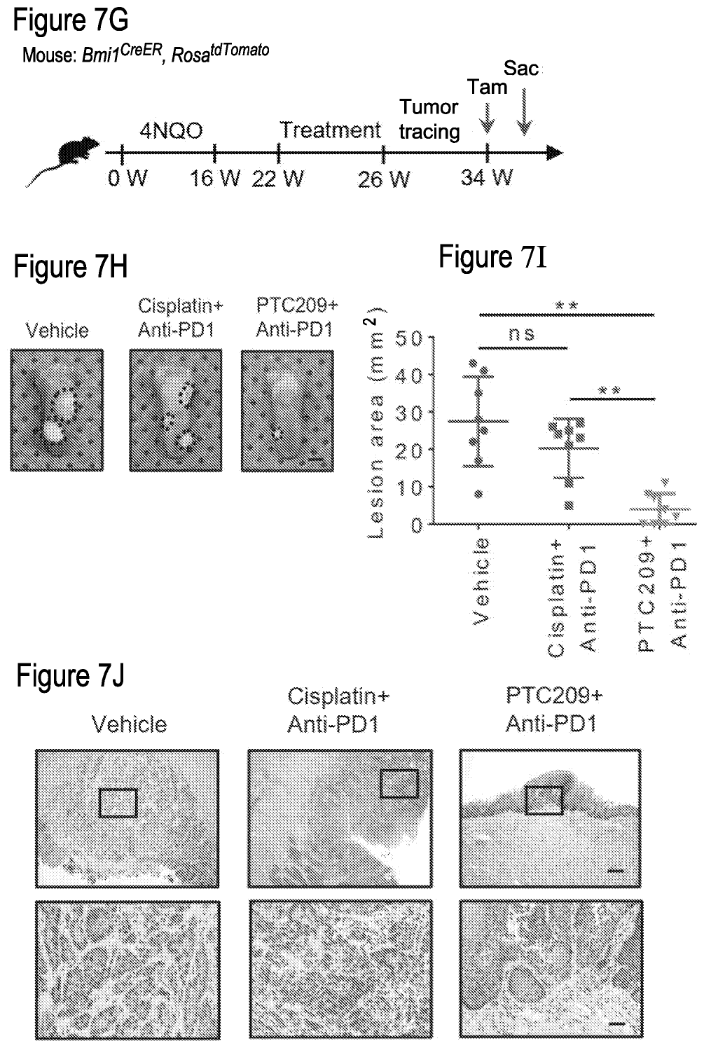

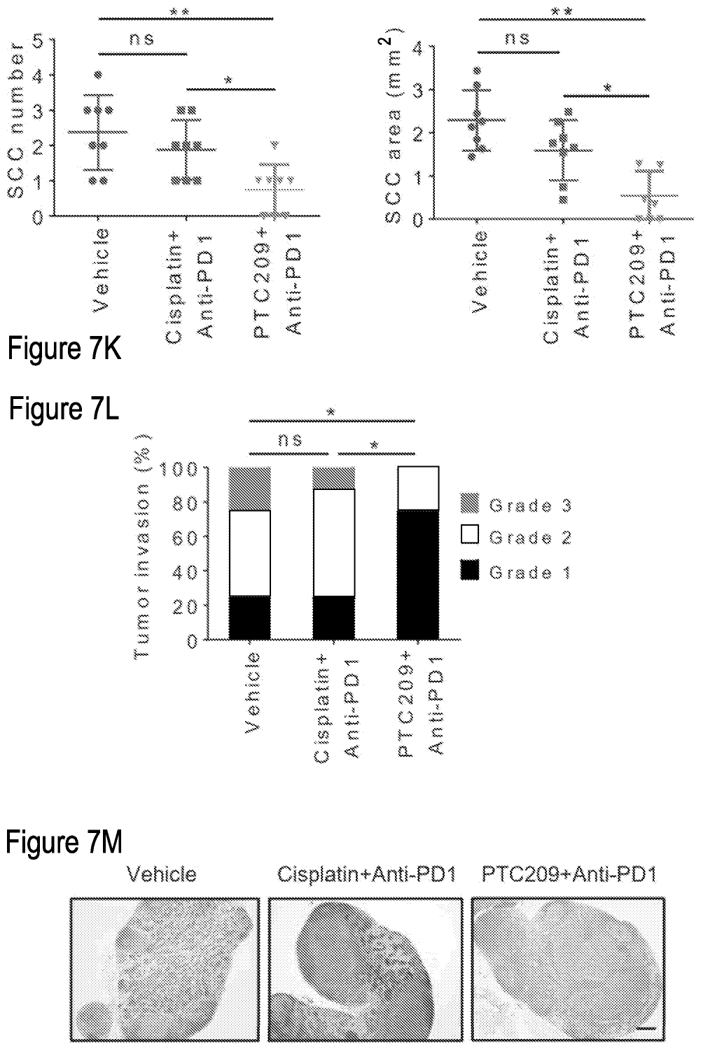

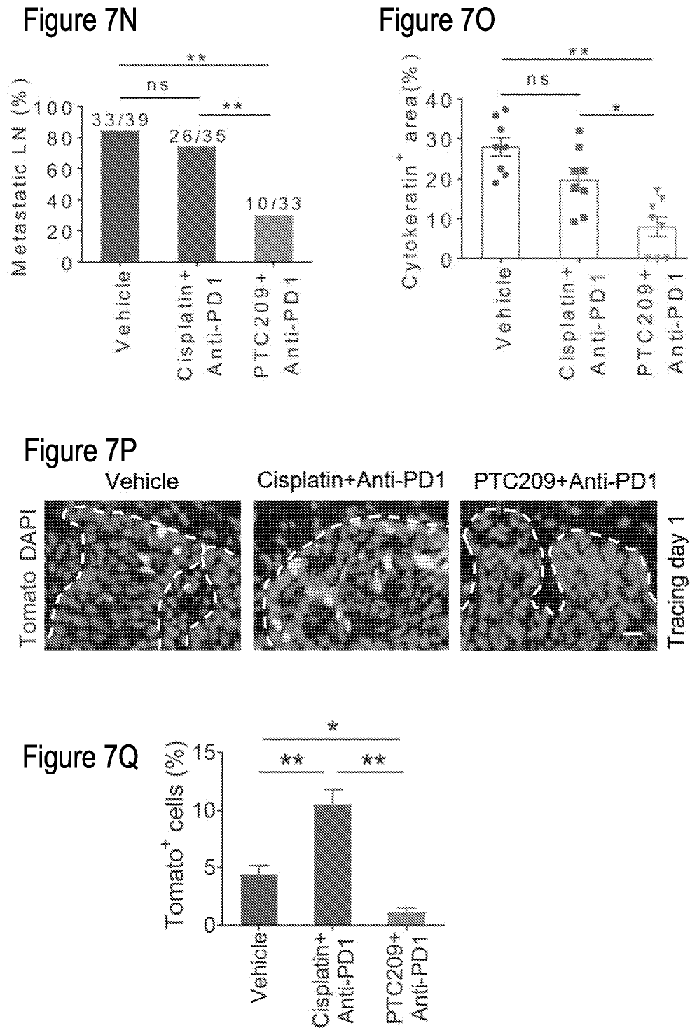

HNSCC frequently relapse although they initially respond to chemotherapies. In vivo labeling revealed that BMI1⁺ CSCs were enriched after anti-PD1 plus cisplatin while PTC209 plus anti-PD1 efficiently eliminated BMI1⁺ CSCs. To determine whether BMI1⁺ CSCs were responsible for HNSCC relapse, in vivo lineage tracing was performed. Tumor-bearing Bmi1^{CreER};Rosa^{tdTomato} mice were treated with anti-PD1 plus cisplatin, anti-PD1 plus PTC209, or control vehicle for 4 weeks and then given tamoxifen to trace Tomato+ cells derived from BMI1⁺ CSCs in HNSCC over a period of 4 weeks (FIG. 7A). In vivo lineage tracing revealed that more than 70% of tumor cells were Tomato⁺ cells in HNSCC treated with anti-PD1 plus cisplatin, which were similar to HNSCC treated with control vehicle. In contrast, Tomato⁺ tumor cells were sparsely present or not detected in regressed HNSCC upon anti-PD1 plus PTC209 (FIGS. 7B and 7C), suggesting that enriched BMI1⁺ CSCs are responsible for HNSCC relapse. Although anti-tumor effects between these two combination treatments did not show a significant difference, a trend was observed that HNSCC treated by anti-PD1 plus PTC209 might have less HNSCC relapse compared with anti-PD1 plus cisplatin (FIGS. 7D-7F).

To further confirm whether HNSCC treated with anti-PD1 plus PTC209 had less relapse compared with anti-PD1 plus cisplatin, the combination treatment was extended for additional 4 weeks in order to obtain recurrent HNSCC (FIG. 7G). Compared with anti-PD1 plus cisplatin, anti-PD1 plus PTC209 had much smaller lesion areas, and even in some cases lesions were undetectable (FIGS. 7H and 7I). Following a prolonged observation, histological analysis found that there was no difference in the numbers, areas and invasion grades of HNSCC between the treatment with control vehicle and anti-PD1 plus cisplatin, thereby validating that HNSCC treated with anti-PD1 plus cisplatin relapsed (FIGS. 7J-7L). In contrast, the inhibition of HNSCC growth by anti-PD1 plus PTC209 was sustained compared with anti-PD1 plus cisplatin, and tumors could not be detected in 3 of the 8 mice after anti-PD1 plus PTC209 (FIGS. 7J-7L). The lymph node metastasis is an important predictor for the relapse and prognosis of HNSCC patients. Furthermore, immunostaining with anti-PCK revealed that only 30% of lymph nodes had metastatic tumor cells in mice treated with anti-PD1 plus PTC209, whereas 74% of lymph nodes had metastatic tumor cells in mice treated with anti-PD1 plus cisplatin (FIGS. 7M-7O). Of note, because the majority of mice treated with anti-PD1 plus cisplatin were very weak due to recurrent HNSCC, these mice needed to be euthanized which did not allow long-term in vivo tracing of the fate of BMI1⁺ CSCs. In vivo labeling BMI1⁺ CSCs revealed that anti-PD1 plus PTC209 efficiently eliminated BMI1⁺ CSCs in regressed HNSCC while BMI1⁺ CSCs remained in recurrent HNSCC treated with anti-PD1 plus cisplatin (FIGS. 7P and 7Q).

### DISCUSSION

Treating or preventing recurrent and metastatic HNSCC remains a great therapeutic challenge regardless of the promising progress in immune checkpoint therapy. In this study, it is showed that the combination treatment of anti-PD1 and cisplatin enriched BMI1⁺ CSCs, although the combination could inhibit HNSCC growth by recruiting CD8⁺ T cells. Not surprisingly, dwindling HNSCC after treatment with anti-PD1 plus cisplatin regrew and eventually relapsed. In contrast, the combination treatment of anti-PD1 and PTC209 not only potently inhibited HNSCC invasive growth, but also significantly reduced HNSCC relapse and lymph node metastasis compared with anti-PD1 plus cisplatin. Mechanistically, while BMI1 inhibition destroyed CSCs, it also induced tumor cell-intrinsic immune responses and augmented PD1 blockade to kill non-stem tumor cells in HNSCC by recruiting and activating CD8⁺ T cells (FIG. 14I). Given the fact that a large number of PD1 blockade-based combination therapies are currently in clinical trials, these preclinical studies suggest that targeting BMI1 might be a new strategy of the combination therapy in order to effectively inhibit metastatic tumor growth and prevent relapse.

HNSCC has an immunosuppressive tumor microenvironment with low tumor-infiltrating lymphocytes. Previous studies have shown that cisplatin could enhance antitumor immunity by increasing the expression of antigen-processing machinery components or impair anti-tumor immunity by inducing the expression of PD-L1. In results presented herein, cisplatin did collaborate with anti-PD1 to recruit CD8⁺ T cells into HNSCC although cisplatin alone could not. However, it is found that anti-PD1 plus cisplatin could not effectively kill BMI1⁺ CSCs based on in vivo lineage tracing. Supporting these results, it was recently reported that CSCs selectively acquired the expression of CD80 to inhibit cytotoxic T cell activity. Interestingly, CD80 was highly expressed in BMI1⁺ CSCs based on RNA-seq results. Therefore, it is possible that CSCs in HNSCC might be intrinsically resistant to CD8⁺ T cell killing. Although conventional therapy combined with PD-1 blockade has been approved for treating HNSCC, the durable response is limited, indicating that PD-1 blockade combined with the conventional therapy may be unable to eliminate CSCs in HNSCC.

It is demonstrated that combination therapy of BMI1 inhibitor and anti-PD1 effectively inhibited tumor growth metastasis by eliminating CSCs. Although use of the BMI1 inhibitor alone also suppressed HNSCC growth and metastasis, it was not as effective as the combination therapy. Previously, it has been reported that PTC209 inhibited colorectal tumor growth and reduced the frequency of CSCs in mouse xenograft models. PTC-209 has also been shown to inhibit xenografted HNSCC growth. However, because these studies transplanted human tumor cells into immunodeficient mice, the potential impact of anti-tumor immune responses could not be observed. In the present disclosure, it is found unexpectedly that BMI1 inhibitor not only inhibited CSC self-renewal, but also activated CSC-intrinsic immune response. These results showed that CD8⁺ T cells recruited to tumor tissues were significantly increased by PTC209 treatment. BMI1 inhibitor could boost the immunotherapy effect of anti-PD1 in addition to targeting BMI1⁺ CSCs, thereby sensitizing non-stem tumor cells to immunotherapy-induced apoptosis.

SCC cells frequently metastasize to cervical lymph nodes which are enriched with immune cells, indicating that immune evasion plays a critical role in HNSCC progression and metastasis. Growing evidence demonstrates that targeting tumor cell-intrinsic genetic and epigenetic alterations is crucial to unleash anti-tumor immunity, thereby inhibiting tumor growth and metastasis. Inhibition of PRC2-mediated histone H3 lysine 27 trimethylation leads to increased responses to cancer immunotherapy. BMI1 is a core component of the PRC1 that mediates gene silencing via H2Aub. PRC1 also cooperates with PRC2 to control chromatin compaction and repress gene expression. BMI1, as an important epigenetic factor, regulates cancer invasive growth and progression in addition to cancer stemness. BMI1 is also associated with DNA damage response and repair. BMI1 ablation impairs the recruitment of DNA repair factors to DSBs which are dependent on ubiquitin signaling, thereby promoting DNA damage. Here, it is found that BMI1 inhibition induced chemokines by two interconnected mechanisms: 1) stimulating cGAS-STING signaling to activate IRF3-mediated transcription by induction of DNA damage, and 2) erasing repressive H2AUb markers epigenetically. It is observed that pH2A.X, a known hallmark of DNA double strand break and DNA damage response activation, was increased in SCC cells upon BMI1 inhibition in vitro and in vivo. Consistent with ongoing DNA damage, cytosolic dsDNA was accumulated which subsequently activated the STING-TBK1-IRF3 pathway to induce the expression of the type I IFN chemokines (CCL5, CXCL9, CXCL10, and CXCL11). The removal of repressive H2Aub marks on the promoters of type I IFN chemokines may further facilitate the trans activation of IRF3 upon BMI1 inhibition.

While Tomato⁺ BMI1⁺ CSCs might represent a rare population of CSCs in HNSCC which have high BMI1 transcription, it has been previously found that the basal level of BMI1 was also increased in non-stem tumor cells. Immunostaining found that human HNSCC tumor samples had a more broad expression pattern because BMI1 proteins could also be post-translationally regulated. Therefore, BMI1 inhibition in these tumor cells should also intrinsically activate their immune response and recruit CD8⁺ T cells. Targeting BMI1 could also sensitize non-stem tumor cells to anti-PD1 by recruiting CD8⁺ T cells in addition to purging CSCs. The combination of PD1 blockade and BMI1 inhibition not only inhibited metastatic HNSCC growth, but also efficiently prevented HNSCC relapses. PTC596, an analog of PTC209 has been in clinical trials to treat advanced solid tumors. The phase 1 study suggested that PTC596 is tolerable with manageable gastrointestinal side effects. In the future, it will be interesting to perform a clinical trial to determine whether anti-PD1 and PTC596 collaboratively inhibit human HNSCC growth and lymph node metastasis. Taken together, the results presented herein have important implications in developing a new combination treatment for advanced cancer by targeting CSCs and activating tumor cell-intrinsic immune responses.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating cancer in a patient in need thereof, comprising the steps of
i) administering to said patient an agent that blocks signaling through programmed cell death protein 1 (PD-1); and
ii) administering to said patient an agent that reduces expression or function of B-cell-specific Moloney murine leukemia virus integration site 1 (BMI-1).

2. The method of claim 1, wherein said cancer is head and neck squamous cell carcinoma, melanoma, lymphoma, leukemia, breast cancer, ovarian cancer, colon cancer, cervical cancer, prostate cancer, nasopharyngeal carcinoma, non-Hodgkin lymphoma, small cell lung cancer, or non-small cell lung cancer, or any combination thereof.

3. The method of claim 1, wherein said method reduces metastasis of said cancer in said patient.

4. The method of claim 1, wherein said method reduces the number of BMI-1⁺ cancer stem cells.

5. The method of claim 1, wherein said agent that blocks signaling through PD-1 is anti-PD-1 antibody or antibody that binds a ligand of PD-1.

6. The method of claim 5, wherein said anti-PD-1 antibody is selected from the group consisting of nivolumab, pembrolizumab, cemiplimab, avelumab, durvalumab, and atezolizumab.

7. The method of claim 5, wherein said ligand of PD-1 is PD-L1 or PD-L2.

8. The method of claim 1, wherein said agent that reduces expression or function of BMI-1 is a BMI-1 inhibitor or small interfering RNA (siRNA).

9. The method of claim 8, wherein said BMI-1 inhibitor is PTC209, PTC596, PRT4165, PTC-209 HBr, or PTC-028.

10. The method of claim 1, wherein said agent of (i) is administered to the patient before, after, or concurrently as said agent of (ii) is administered to the patient.

11. A method of increasing anti-tumor T cell activities in a patient having a cancer, comprising the steps of
i) administering to said patient an agent that blocks signaling through programmed cell death protein 1 (PD-1); and
ii) administering to said patient an agent that reduces expression or function of B-cell-specific Moloney murine leukemia virus integration site 1 (BMI-1).

12. The method of claim 11, wherein said cancer is head and neck squamous cell carcinoma, melanoma, lymphoma, leukemia, breast cancer, ovarian cancer, colon cancer, cervical cancer, prostate cancer, nasopharyngeal carcinoma, non-Hodgkin lymphoma, small cell lung cancer, or non-small cell lung cancer, or any combination thereof.

13. The method of claim 11, wherein said anti-tumor T cell activities are mediated by CD8⁺ T cells.

14. The method of claim 11, wherein said agent that blocks signaling through PD-1 is anti-PD-1 antibody or antibody that binds a ligand of PD-1.

15. The method of claim 14, wherein said anti-PD-1 antibody is selected from the group consisting of nivolumab, pembrolizumab, cemiplimab, avelumab, durvalumab, and atezolizumab.

16. The method of claim 14, wherein said ligand of PD-1 is PD-L1 or PD-L2.

17. The method of claim 11, wherein said agent that reduces expression or function of BMI-1 is a BMI-1 inhibitor or small interfering RNA (siRNA).

18. The method of claim 17, wherein said BMI-1 inhibitor is PTC209, PTC596, PRT4165, PTC-209 HBr, or PTC-028.

19. The method of claim 11, wherein said agent of (i) is administered to the patient before, after, or concurrently as said agent of (ii) is administered to the patient.

20. A method of reducing the number of cancer stem cells in a cancer patient in need thereof, comprising the steps of
i) administering to said patient an agent that blocks signaling through programmed cell death protein 1 (PD-1); and
ii) administering to said patient an agent that reduces expression or function of B-cell-specific Moloney murine leukemia virus integration site 1 (BMI-1).

21. The method of claim 20, wherein said cancer is head and neck squamous cell carcinoma, melanoma, lymphoma, leukemia, breast cancer, ovarian cancer, colon cancer, cervical cancer, prostate cancer, nasopharyngeal carcinoma, non-Hodgkin lymphoma, small cell lung cancer, or non-small cell lung cancer, or any combination thereof.

22. The method of claim 20, wherein said cancer stem cells are BMI-1⁺.

23. The method of claim 20, wherein said agent that blocks signaling through PD-1 is anti-PD-1 antibody or antibody that binds a ligand of PD-1.

24. The method of claim 23, wherein said anti-PD-1 antibody is selected from the group consisting of nivolumab, pembrolizumab, cemiplimab, avelumab, durvalumab, and atezolizumab.

25. The method of claim 23, wherein said ligand of PD-1 is PD-L1 or PD-L2.

26. The method of claim 20, wherein said agent that reduces expression or function of BMI-1 is a BMI-1 inhibitor or small interfering RNA (siRNA).

27. The method of claim 26, wherein said BMI-1 inhibitor is PTC209, PTC596, PRT4165, PTC-209 HBr, or PTC-028.

28. The method of claim 20, wherein said agent of (i) is administered to the patient before, after, or concurrently as said agent of (ii) is administered to the patient.

29. A composition for treating cancer in a patient, comprising (i) a composition of an agent that blocks signaling through programmed cell death protein 1 (PD-1); and (ii) a composition of an agent that reduces expression or function of B-cell-specific Moloney murine leukemia virus integration site 1 (BMI-1).

30. The composition of claim 29, wherein said agent that blocks signaling through PD-1 is anti-PD-1 antibody or antibody that binds a ligand of PD-1.

31. The composition of claim 30, wherein said anti-PD-1 antibody is selected from the group consisting of nivolumab, pembrolizumab, cemiplimab, avelumab, durvalumab, and atezolizumab.

32. The composition of claim 30, wherein said ligand of PD-1 is PD-L1 or PD-L2.

33. The composition of claim 29, wherein said agent that reduces expression or function of BMI-1 is a BMI-1 inhibitor or small interfering RNA (siRNA).

34. The composition of claim 33, wherein said BMI-1 inhibitor is PTC209, PTC596, PRT4165, PTC-209 HBr, or PTC-028.

35. A therapeutic combination of compositions formulated for treating cancer in a patient, comprising (i) a composition of an agent that blocks signaling through programmed cell death protein 1 (PD-1); and (ii) a composition of an agent that reduces expression or function of B-cell-specific Moloney murine leukemia virus integration site 1 (BMI-1).

36. The therapeutic combination of compositions of claim 35, wherein said agent that blocks signaling through PD-1 is anti-PD-1 antibody or antibody that binds a ligand of PD-1.

37. The therapeutic combination of compositions of claim 36, wherein said anti-PD-1 antibody is selected from the group consisting of nivolumab, pembrolizumab, cemiplimab, avelumab, durvalumab, and atezolizumab.

38. The therapeutic combination of compositions of claim 36, wherein said ligand of PD-1 is PD-L1 or PD-L2.

39. The therapeutic combination of compositions of claim 35, wherein said agent that reduces expression or function of BMI-1 is a BMI-1 inhibitor or small interfering RNA (siRNA).

40. The therapeutic combination of compositions of claim 39, wherein said BMI-1 inhibitor is PTC209, PTC596, PRT4165, PTC-209 HBr, or PTC-028.

41. The compositions of claim 29 or 35 for use in treating cancer in a patient.

42. The compositions for use according to claim 41, wherein the cancer is head and neck squamous cell carcinoma, melanoma, lymphoma, leukemia, breast cancer, ovarian cancer, colon cancer, cervical cancer, prostate cancer, nasopharyngeal carcinoma, non-Hodgkin lymphoma, small cell lung cancer, non-small cell lung cancer, or any combination thereof.

43. The compositions for use according to claim 41, wherein treatment with said composition reduces metastasis of said cancer in said patient.

44. The compositions for use according to claim 41, wherein treatment with said composition reduces the number of BMI-1⁺ cancer stem cells in said patient.

45. The compositions for use according to claim 41, wherein said composition of (i) is administered to the patient before, after, or concurrently as said composition of (ii) is administered to the patient.


SEQUENCE LISTING (35 sequences)

SEQ ID NO: 1 - DNA, 20 bp, Artificial Sequence
  human GAPDH forward primer
aacgggaagcttgtcatcaa

SEQ ID NO: 2 - DNA, 20 bp, Artificial Sequence
  human GAPDH reverse primer
tggactccacgacgtactca

SEQ ID NO: 3 - DNA, 20 bp, Artificial Sequence
  human CCL5 forward primer
cagtggcaagtgctccaacc

SEQ ID NO: 4 - DNA, 25 bp, Artificial Sequence
  human CCL5 reverse primer
ccatcctagctcatctccaaagagt

SEQ ID NO: 5 - DNA, 22 bp, Artificial Sequence
  human CXCL9 forward primer
gtggtgttcttttcctcttggg

SEQ ID NO: 6 - DNA, 21 bp, Artificial Sequence
  human CXCL9 reverse primer
acagcgaccctttctcactac

SEQ ID NO: 7 - DNA, 20 bp, Artificial Sequence
  human CXCL10 forward primer
gcaagccaattttgtccacg

SEQ ID NO: 8 - DNA, 25 bp, Artificial Sequence
  human CXCL10 reverse primer
acatttccttgctaactgctttcag

SEQ ID NO: 9 - DNA, 21 bp, Artificial Sequence
  human CXCL11 forward primer
cagaattccactgcccaaagg

SEQ ID NO: 10 - DNA, 23 bp, Artificial Sequence
  human CXCL11 reverse primer
gtaaactccgatggtaaccagcc

SEQ ID NO: 11 - DNA, 21 bp, Artificial Sequence
  human IFNb forward primer
gccatcagtcacttaaacagc

SEQ ID NO: 12 - DNA, 22 bp, Artificial Sequence
  human IFNb reverse primer
gaaactgaagatctcctagcct

SEQ ID NO: 13 - DNA, 20 bp, Artificial Sequence
  mouse GAPDH forward primer
ctcatgaccacagtccatgc

SEQ ID NO: 14 - DNA, 20 bp, Artificial Sequence
  mouse GAPDH reverse primer
cacattgggggtaggaacac

SEQ ID NO: 15 - DNA, 18 bp, Artificial Sequence
  mouse CCL5 forward primer
caccactccctgctgctt

SEQ ID NO: 16 - DNA, 18 bp, Artificial Sequence
  mouse CCL5 reverse primer
acacttggcggttccttc

SEQ ID NO: 17 - DNA, 21 bp, Artificial Sequence
  mouse CXCL9 forward primer
ttttgggcatcatcttcctgg

SEQ ID NO: 18 - DNA, 25 bp, Artificial Sequence
  mouse CXCL9 reverse primer
gaggtctttgagggatttgtagtgg

SEQ ID NO: 19 - DNA, 20 bp, Artificial Sequence
  mouse CXCL10 forward primer
cttctgaaaggtgaccagcc

SEQ ID NO: 20 - DNA, 20 bp, Artificial Sequence
  mouse CXCL10 reverse primer
gtcgcacctccacatagctt

SEQ ID NO: 21 - DNA, 23 bp, Artificial Sequence
  mouse CXCL11 forward primer
aacaggaaggtcacagccatagc

SEQ ID NO: 22 - DNA, 20 bp, Artificial Sequence
  mouse CXCL11 reverse primer
tttgtcgcagccgttactcg

SEQ ID NO: 23 - DNA, 21 bp, Artificial Sequence
  mouse IFNb forward primer
ggtggaatgagactattgttg

SEQ ID NO: 24 - DNA, 17 bp, Artificial Sequence
  mouse IFNb reverse primer
aggacatctcccacgtc

SEQ ID NO: 25 - DNA, 21 bp, Artificial Sequence
  mouse IFNb forward primer
ggtggaatgagactattgttg

SEQ ID NO: 26 - DNA, 17 bp, Artificial Sequence
  mouse IFNb reverse primer
aggacatctcccacgtc

SEQ ID NO: 27 - DNA, 25 bp, Artificial Sequence
  human CCL5 forward primer
caccattggtgcttggtcaaagagg

SEQ ID NO: 28 - DNA, 25 bp, Artificial Sequence
  human CCL5 reverse primer
gcagtagcaatgaggatgacagcga

SEQ ID NO: 29 - DNA, 20 bp, Artificial Sequence
  human CXCL9 forward primer
tgtgccaaaggctatcagtg

SEQ ID NO: 30 - DNA, 21 bp, Artificial Sequence
  human CXCL9 reverse primer
cagatccaagggaatttctgc

SEQ ID NO: 31 - DNA, 23 bp, Artificial Sequence
  human CXCL10 forward primer
taggtttacctataaaggatgaa

SEQ ID NO: 32 - DNA, 24 bp, Artificial Sequence
  human CXCL10 reverse primer
actcgaaggtattatttattgtag

SEQ ID NO: 33 - DNA, 20 bp, Artificial Sequence
  human CXCL11 forward primer
ggttttcacagtgctttcac

SEQ ID NO: 34 - DNA, 20 bp, Artificial Sequence
  human CXCL11 reverse primer
tttccctctttgagtcatgc

SEQ ID NO: 35 - DNA, 57 bp, Artificial Sequence
  BMI1 specific shRNA lentiviral plasmid
ccggcctaatactttccagattgatctcgagatcaatctggaaagtattaggttttt